SEQUENCE LISTING (11 sequences)

SEQ ID NO: 1 - protein, 9 aa, synthetic construct
RKKRRQRRR

SEQ ID NO: 2 - protein, 10 aa, synthetic construct
PLILLRLLRG

SEQ ID NO: 3 - protein, 10 aa, synthetic construct
PLIYLRLLRG

SEQ ID NO: 4 - protein, 17 aa, synthetic construct
  SITE 2: note = 6-aminohexanoic acid
  SITE 16: note = 6-aminohexanoic acid
  MOD_RES 5: note = beta-Alanine
  MOD_RES 14: note = beta-Alanine
RXRRXRRYQFLIRXRXR

SEQ ID NO: 5 - protein, 10 aa, synthetic construct
  VARIANT 6: note = modified arginine, the side chain of which comprises an amidino functional group, guanidino functional group, or amidine-like motif
  VARIANT 9: note = modified arginine, the side chain of which comprises an amidino functional group, guanidino functional group, or amidine-like motif
PLIYLXLLXG

SEQ ID NO: 6 - protein, 9 aa, synthetic construct
RRRRRRRRR

SEQ ID NO: 7 - protein, 16 aa, synthetic construct
RQIKIWFQNRRMKWKK

SEQ ID NO: 8 - protein, 22 aa, synthetic construct
MVRRFLVTLRIRRACGPPRVRV

SEQ ID NO: 9 - protein, 11 aa, synthetic construct
  SITE 1: note = 6-aminohexanoic acid
  SITE 7: note = (S)-2-amino-3-(4-carbamimidoylbenzamido)propanoic acid
  SITE 10: note = (S)-2-amino-3-(4-carbamimidoylbenzamido)propanoic acid
XPLIYLXLLXG

SEQ ID NO: 10 - protein, 11 aa, synthetic construct
  SITE 1: note = 6-aminohexanoic acid
  SITE 7: note = (S)-2-amino-3-(1H-benzo[d]imidazole-5/6-carboxamido )propanoic acid
  SITE 10: note = (S)-2-amino-3-(1H-benzo[d]imidazole-5/6-carboxamido )propanoic acid
XPLIYLXLLXG

SEQ ID NO: 11 - protein, 11 aa, synthetic construct
  SITE 1: note = 6-aminohexanoic acid
  SITE 7: note = (S)-2-amino-3-(3-carbamimidoylbenzamido)propanoic acid
  SITE 10: note = (S)-2-amino-3-(3-carbamimidoylbenzamido)propanoic acid
XPLIYLXLLXG

(19) United States
(12) Patent Application Publication
(10) Pub. No.: US 2026/0226103 A1
(43) Pub. Date: Aug. 6, 2026

(54) MODIFIED AMINO ACIDS AND USES THEREOF

(71) Applicant: UNIVERSITY OF STRATHCLYDE, Glasgow (GB)

(72) Inventors: Glenn BURLEY, Glasgow (GB); Jack ROBERTSON, Dundee (GB); Fergus Stewart MCWHINNIE, Barrhead (GB)

(21) Appl. No.: 18/876,684

(22) PCT Filed: Jun. 22, 2023
(86) PCT No.: PCT/GB2023/051645

§ 371 Date: Dec. 19, 2024

(30) Foreign Application Priority Data
Jun. 23, 2022 (GB) 2209228.2

(51) Int. Cl. C07K 7/08; A61K 38/00; A61K 47/64; C07K 7/06

(52) U.S. Cl. CPC C07K 7/08; A61K 47/64; C07K 7/06; A61K 38/00; C07K 2319/10

(57) ABSTRACT

The present disclosure relates to modified amino acids which have been modified to modulate their physicochemical properties. The present disclosure further relates to the use of such modified amino acids to provide peptides, in particular, cell penetrating peptides with modified physicochemical properties.

### RELATED APPLICATIONS

This application is a 35 U.S.C. § 371 national stage of PCT Application No. PCT/GB2023/051645, filed on Jun. 22, 2023, which claims priority from United Kingdom Patent Application No. 2209228.2, filed on Jun. 23, 2022, the contents of which are incorporated herein by reference. The above-referenced PCT International Application was published in the English language as International Publication No. WO 2023/247968 A1 on Dec. 28, 2023.

### STATEMENT REGARDING ELECTRONIC FILING OF A SEQUENCE LISTING

A Sequence Listing in XML format, entitled “9013-236_ST26.xml,” 16,700 bytes in size, generated on Apr. 17, 2026, and filed herewith, is hereby incorporated by reference into the specification for its disclosures.

### FIELD

The present disclosure relates to modified amino acids which have been modified to modulate their physicochemical properties. The present disclosure further relates to the use of such modified amino acids to provide peptides, in particular, cell penetrating peptides with modified physicochemical properties.

### BACKGROUND

The cell membrane can be a formidable barrier for the development of small molecule and biologic drug entities. Composed of phospholipids, membrane-bound glycoproteins and fluidic modifiers (e.g., cholesterol), cell membranes are responsible for maintaining a non-equilibrium state within the cell relative to its extracellular environment. Whereas recent progress in the drug discovery arena has focused on understanding how the physicochemical properties of small molecules influence cell uptake, equivalent design criteria for the development of larger molecular weight biomacromolecular (biologic) drugs, such as therapeutic proteins and oligonucleotides, can be more challenging to achieve. As a result, the development of delivery vehicles, either conjugated to a drug of interest or used as part of a multi-component nanovector (e.g., nanoparticles, liposomal formulations), can be used to expand the types of biomolecules which can be targeted by biologics and non-Lipinski compliant small molecules.

Cell penetrating peptides (CPPs) are one example of a class of delivery vehicles that can be used to deliver agents to a cell. Cell penetrating peptides have been used as biotechnological tools, however the emergence of toxicity associated with CPPs has hampered their use in clinical applications (Guidotti et al, Cell-Penetrating Peptides: From Basic Research to Clinics. Trends Pharmacol. Sci. 2017, 38, 406-424). Therefore, one challenge is the development of CPP scaffolds, which maintain high levels of uptake and cellular distribution, but with reduced levels of toxicity.

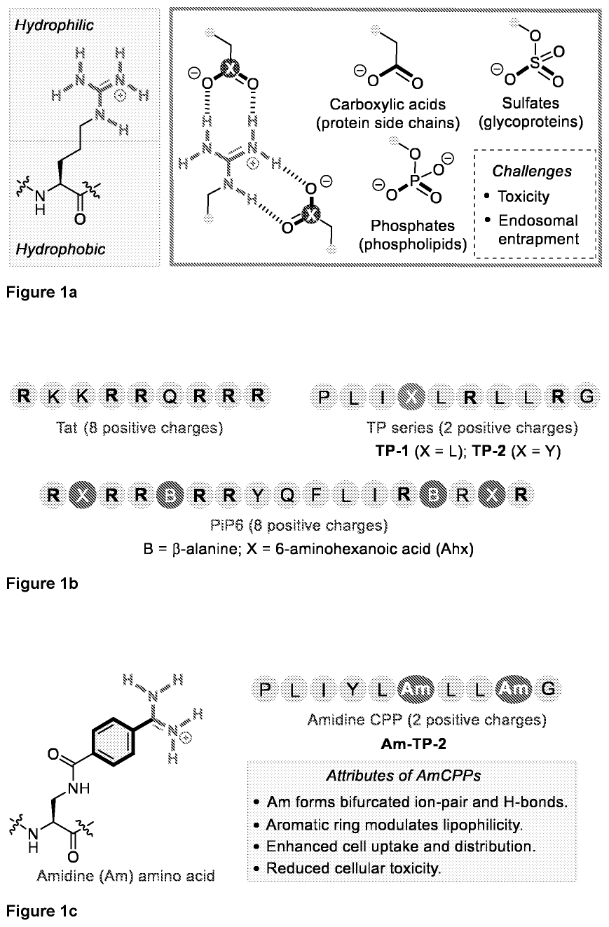

A structural hallmark of the majority of CPPs is that their sequences typically comprise multiple arginine (Arg) residues (see, for example, FIG. 1a). The positively-charged guanidinium sidechain of Arg residues form stable bifurcated non-covalent ion-paired interactions with negatively-charged phospholipids and sulfonated glycoproteins. In combination with Arg residues, a balance between positive charge and hydrophobicity is typically needed for efficient translocation of cell membranes and to evade endosomal entrapment. However, the use of multiple positively-charged Arg residues in CPPs can also be a source of toxicity. As a result, extensive efforts have focused on identifying new CPPs, primarily using naturally-occurring amino acids, to mitigate the toxicity associated with high Arg content whilst still maintaining the ability to enhance the uptake of therapeutic cargoes. Some examples are shown in FIG. 1b (Dinca, A.; Chien, W.-M.; Chin, M. T. Intracellular Delivery of Proteins with Cell-Penetrating Peptides for Therapeutic Uses in Human Disease. Int.l J. Mol. Sci. 2016, 17, 263; Park, S. E.; Sajid, M. I.; Parang, K.; Tiwari, R. K. Cyclic Cell-Penetrating Peptides as Efficient Intracellular Drug Delivery Tools. Mol. Pharmaceutics 2019, 16, 3727-3743; and Silva, S.; Almeida, A. J.; Vale, N. Combination of Cell-Penetrating Peptides with Nanoparticles for Therapeutic Application: A Review. Biomolecules 2019, 9).

Some examples of modified and/or non-natural amino acids are disclosed in JP06495714B2 (Makoto et al), WO2020210916A1 (Guay et al) and U.S. Ser. No. 10/253,099B2 (Strieker et al). Further examples may be found in Behrouz et al, Tetrahedron Lett. Vol 81 2021, page 153342, Hosseini et al, Molecular Pharmacology, Vol. 80(4), 2011, pages 585-597, WO2022014704A1 (Santen Pharmaceutical Co Ltd), WO96/40743 (Cor Therapeutics), Zhang et al, Bioorganic & Medicinal Chemistry, Vol. 10(11), 2002, pages 3401-3413, WO2022/129047 (H Lundbeck AS), WO99/31061A1 (Merck & Co. Inc.), WO2009/051397 (Choongwae Pharma Corp.), WO2020/006315A1 (Pliant Therapeutics Inc.), EP2995612A1 (Universite de Strasbourg). However, there remains a need to identify further modified amino acids and cell penetrating peptides that address some of the problems noted in the art.

### SUMMARY

The present disclosure is based on the finding that modified amino acids can be used to provide peptides (e.g. cell penetrating peptides) with improved properties. In particular, it has been identified that targeted modifications to modulate the physicochemical properties of side chains of certain amino acids can lead to improved cellular uptake, cellular distribution and/or a reduced toxicity for cell penetrating peptides comprising residues derived from these modified amino acids.

Indeed, as discussed above, many cell penetrating peptides typically comprise multiple arginine residues. A novel class of modified amino acids has been identified that aims to address some of the problems associated with the use of multiple arginine residues. In particular, the present inventors have identified a cohort of modified amino acids which aim to mimic the naturally occurring guandinium group present in arginine residues but which have been specifically modified to modulate certain physicochemical properties (e.g. basicity, hydrophobicity, amphipathicity, pKa, lipophilicity, etc.). For example, the modified amino acids disclosed herein may comprise an increased lipophilicity and/or hydrophobicity relative to arginine. These modified amino acids have been found to provide enhanced cellular uptake, cellular distribution and/or reduced toxicity when used to replace arginine in a number of cell penetrating peptides.

Specifically, it has been recognised that targeted modifications to the side chain of arginine can modulate the physicochemical properties and increase the “drug-like” properties of this amino acid and/or cell penetrating peptides comprising an amino acid residue derived from this amino acid. For example, a bio-isosteric replacement of the guanidinium group of an arginine with an amidine group (or an amidine-mimetic group) has been found to modulate the lipophilicity and/or basicity of the amino acid. Alternatively, other targeted modifications can be made to the side chain of arginine to modulate the lipophilicity and/or basicity of the guanidine group at the terminus of the side chain. The use of such modified amino acids as replacements to arginine in CPPs can significantly enhance cellular uptake and cellular distribution and may show no adverse effect on toxicity.

By way of example, the modified amino acids have been specifically modified to have a lower pKa than arginine (which has a pKa of approximately 12.5 at 25° C.). Therefore, the modified amino acids of the disclosure may have a pKa of less than about 12.5 at 25° C. By way of further example, the modified amino acids of the disclosure may have a pKa between about 4 and about 12, or between about 5 and about 11, at 25° C.

Additionally or alternatively, the modified amino acids have been specifically modified to increase their hydrophobicity in comparison to arginine (which has a Log D or c Log D of approximately −3.5 at a pH of 7.4). As such, the modified amino acids of the disclosure may have a Log D or c Log D of greater than about −3.5 at a pH of 7.4. By way of further example, the modified amino acids may have a Log D or c Log D of between about −3 and about 2, or between about −2 and about 1, at a pH of 7.4. As used herein, a Log D value is a distribution coefficient which can be used to provide a measure of the lipophilicity of ionizable compounds at a particular pH. As used herein, in a c Log D value, the “c” indicates that the value is calculated.

As used herein, a cell penetrating peptide (CPP) may refer to a peptide that can facilitate cellular uptake and/or distribution of an agent of interest (sometimes referred to as the “cargo” or “payload”). Typically, a cell penetrating peptide may comprise between about 2 and 100 amino acid residues, such as between about 5 and 50 or about 7 and 20 amino acid residues. In some examples, the cell penetrating peptide may comprise between about 2 and 30 amino acid residues.

Cell penetrating peptides may be broadly classified into several categories, including cationic, amphipathic, membranotropic and hydrophobic. Without being bound by theory, it is believed that the hydrophilicity and hydrophobicity are correlated to their different ways of interacting with the membrane bilayer. Cationic CPPs may be rich in arginine, lysine and histidine residues, and particularly arginine residues. Therefore, the modified amino acids as described herein may find particular application in cationic CPPs. As used herein, cationic CPPs carry a net positive charge. Without being bound by theory, this net positive charge is understood to be important to their ability to cross cellular membranes. The amino acids disclosed here may find particular utility in cationic CPPs as they may provide that charge whilst showing a modulated basicity and/or pKa.

The agent of interest may be associated with the peptide either via a chemical linkage (e.g. covalent bond) and/or via non-covalent interactions using methods known in the art. The skilled person will appreciate that the linker acts to tether the agent of interest to the cell penetrating peptide whilst also allowing both of these portions to perform their respective functions and/or bind to their targets. In particular, the linker may act to tether the agent of interest to the cell penetrating peptide whilst also mitigating the possibility of the cell penetrating peptide disrupting, interfering with and/or inhibiting: (i) the binding of the agent of interest to any target; and/or (ii) the activity or intended function of the agent of interest. Additionally or alternatively, the linker may act to tether the agent of interest to the cell penetrating peptide whilst also mitigating the possibility of the agent of interest disrupting, interfering with and/or inhibiting the binding and/or interactions of the cell penetrating peptide (e.g. its function in modulating, facilitating and/or promoting the delivery of the agent of interest into the cell).

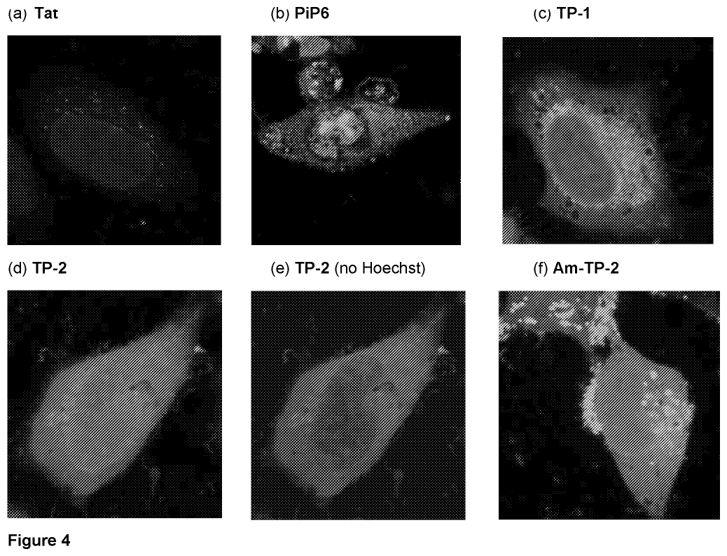

In some examples, the agent of interest may be associated with the cell penetrating peptide by a covalent linkage which may comprise a moiety comprising an ester, an amide, a disulfide or a thioester group. The linker may be derived from an amino substituted carboxylic acid, such as an amino substituted C₁ to C₂₀ alkyl carboxylic acid (e.g. an amino substituted C₁ to C₁₀ alkyl carboxylic acid). In some examples, the linker may be derived from 6-aminohexanoic acid. By way of example, the agent of interest may be associated with the peptide by way of a maleimide, succindyl ester or isothiocyanate linkage (e.g. such as is shown in FIG. 4B of Jones and Sayers, “Cell entry of cell penetrating peptides: tales of tails wagging dogs”, Journal of Controlled Release, 161(2012), 582-591, the contents of which are incorporated herein by reference. In this figure (and with reference to the present disclosure) the term “Fluor” may represent any agent of interest.

By way of further example, the agent of interest may be associated with the cell penetrating peptide by a covalent linker selected from one of the following structures:

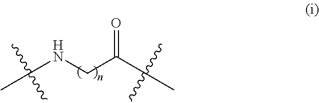

wherein n is from 0 to 10 (e.g. 1, 2, 3, 4, 5, 6, 7, 8, 9 or 10), such as:

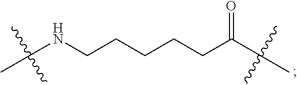

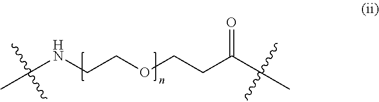

wherein n is from 1 to 10 (e.g. 1, 2, 3, 4, 5, 6, 7, 8, 9, 10), such as:

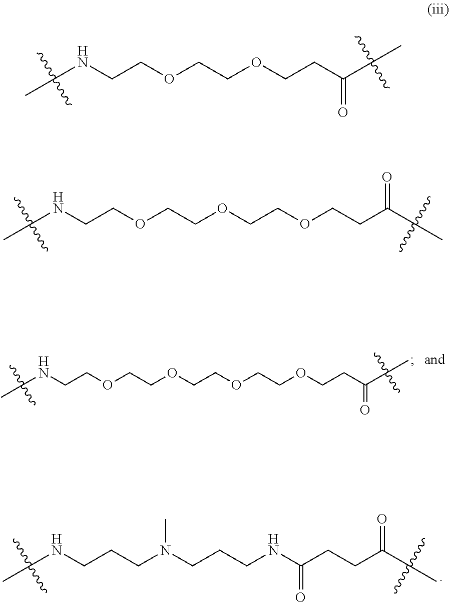 [SMILES: *NCCOCCOCCC(*)=O ; *NCCOCCOCCOCCC(*)=O ; *NCCOCCOCCOCCOCCC(*)=O ; *NCCCN(C)CCCNC(=O)CCC(*)=O]

In each of the structures above, the bonds intersected by the wavy lines indicate the point of attachment of the structure to cell penetrating peptide and the agent of interest.

The agent of interest may be type of entity that requires delivery into a cell and may be for example, a therapeutic agent, a diagnostic agent or a contrast agent. The agent of interest may be a biological molecule (e.g. a nucleic acid-based molecule (e.g. siRNA, antisense oligonucleotide, DNA, plasmid, etc), an antibody, a polysaccharide, a polypeptide or protein). In some examples, the agent of interest may be a particle (e.g. nanosize particle) or a chemical compound.

Further information on cell penetrating peptides and their use in delivering a cargo into a cell may be found in Jones and Sayers, “Cell entry of cell penetrating peptides: tales of tails wagging dogs”, Journal of Controlled Release, 161(2012), 582-591 and Falanga et al, “The world of cell penetrating: the future of medical applications”, Future Medicinal Chemistry, 2020, 12(15), 1431-1446, the contents of which are incorporated herein by reference.

Accordingly, there is described a modified amino acid according to formula (I):

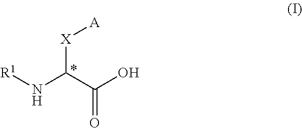

wherein:
   R¹ is H or protecting group;
   X is absent, or is selected from optionally substituted C₁-C₆ alkyl, optionally substituted C₁-C₆ alkyl-NR²(C═O) and optionally substituted —C₁-C₆ alkyl-(C═O)NR²—;
   R² is selected from H, optionally substituted C₁-C₆ alkyl and protecting group; wherein A is selected from:

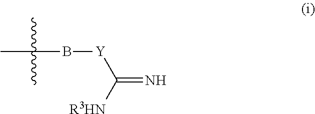

wherein B is selected from optionally substituted aryl, optionally substituted heteroaryl and optionally substituted C₁-C₆ alkyl;
Y is absent or NH;
wherein R³ is selected from H, optionally substituted C₁-C₆ alkyl and protecting group; with the proviso that Y is absent when B is optionally substituted C₁-C₆ alkyl; or
   (ii) a bicyclic fused ring system comprising an amidine-like motif according to the following formula:

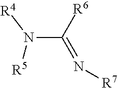

wherein R⁴ and R⁶ are each independently selected from H and a carbon atom that forms part of a backbone of the bicyclic ring system;
R⁵ is selected from H and protecting group; and
R⁷ is a carbon atom that forms part of a backbone of the bicyclic ring system.

In some examples, the at least one modified amino acid does not comprise the following structure:

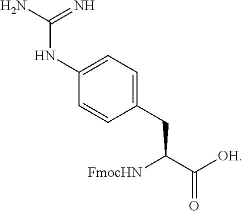

In those examples where A is defined as in point (i) above, the bond intersected by the wavy line represents the covalent bond between group B and X on the parent structure of formula (I). When X is absent, the bond intersected by the wavy line represents the covalent bond between group B and the carbon atom at the starred (*) position in formula (I).

In those examples where A is a bicyclic fused ring system comprising an amidine-like motif as defined in point (ii) above, the bicyclic ring system is joined to the parent structure of formula (I) via a covalent bond between a ring atom on the bicyclic ring system and an atom in group X where present (e.g. the terminal carbon atom of either the optionally substituted C₁-C₆ alkyl, the optionally substituted C₁-C₆ alkyl-NR²(C═O)— or the optionally substituted —C₁-C₆ alkyl-(C═O)NR²—). When X is absent the bicyclic ring system is joined via a covalent bond between a ring atom on the bicyclic ring system and the carbon atom at the starred (*) position in formula (I).

In formula (I) above, the stereochemistry at the starred (*) position may be (R) or (S). In some examples, the amino acid may be provided having a single chiral configuration at this position (e.g. as a single enantiomer or substantially pure single enantiomer). In other examples, the amino acid may be provided as a racemic mixture (e.g. a mixture comprising equimolar amounts of both enantiomers). Amino acids of formula (I) may be provided in substantially a single optical form (e.g. the (+) (dextrorotatory) or (−) (levorotatory) form).

As stated previously, the modified amino acids according to formula (I) may have a pKa of less than about 12.5 at 25° C. By way of further example, the modified amino acids according to formula (I) may have a pKa between about 4 and about 12, or between about 5 and about 11, at 25° C.

Additionally or alternatively, the modified amino acids according to formula (I) may have a Log D or c Log D of greater than about −3.5 at a pH of 7.4. By way of further example, the modified amino acids according to formula (I) may have a Log D or c Log D of between about −3 and about 2, or between about −2 and about 1, at a pH of 7.4.

Additionally or alternatively, the modified amino acids according to formula (I) may comprise a suitable length and/or geometry of chemical moieties in the X and A portions to provide a vector projection of the amidino or guanidino functional group (or of the amidine-like motif) similar to the vector projection of the guanidino group of an arginine amino acid. In particular, as used herein, the vector projection may refer to the direction and distance in space of the amidino or guanidino functional group (or of the amidine-like motif) of the modified amino acid relative to the central carbon atom on the amino acid base structure (e.g. the backbone alpha carbon of the amino acid). In other words, the vector projection may refer to the direction and distance of these groups from the backbone of the amino acid once it has been incorporated into a peptide. As stated above, this vector projection may be similar to that of the guanidino group in arginine relative to the central carbon atom on the amino acid base structure/the backbone of this amino acid once it has been incorporated into a peptide. As will be appreciated by the skilled person, this vector projection may be controlled by an appropriate selection and combination of chemical groups (e.g. those of restricted degrees of freedom such as rings and/or unsaturated groups) and/or chain length (e.g. the number of atoms in a chain and/or in between the amidino or guanidino group (or the amidine-like motif) and the amino acid backbone).

In some examples, the vector projection of the amidino or guanidino functional group (or of the amidine-like motif) may project at least about 7 or at least about 8 Angstroms from the backbone alpha carbon of the amino acid. In preferred examples, the vector projection of the amidino or guanidino functional group (or of the amidine-like motif) may project about 9 Angstroms from the backbone alpha carbon of the amino acid. For example, the vector may project between about 9 Angstroms and about 11 Angstroms from the backbone alpha carbon. In some examples, there may be between about 6 and about 12 linked atoms, between about 7 and about 11 linked atoms, or between about 8 and about 10 linked atoms between the amidino or guanidino functional group (or the amidine-like motif) and the backbone alpha carbon atom of the amino acid functional group. Where an amidino functional group or an amidine-like motif is present, such structures may comprise a hydrogen bond donor group and a hydrogen bond acceptor group placed between about 2 Angstroms and about 6 Angstroms from one another.

In some examples, the modified amino acids may comprise an (S) stereochemistry. Accordingly, in some examples, formula (I) may be represented by the following formula:

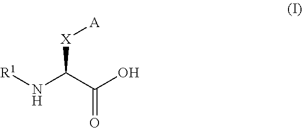

Wherein R¹, X and A are as defined above and hereinafter.

As used herein, the term “alkyl” refers to a straight or branched chain hydrocarbyl group. The chain may be saturated or unsaturated, e.g. in some cases the chain may contain one or more double or triple bonds.

As used herein, “C₁-C₂₀ alkyl” may be selected from straight or branched chain hydrocarbyl groups containing from 1 to 20 carbon atoms. As used herein, “C₁-C₁₀ alkyl” may be selected from straight or branched chain hydrocarbyl groups containing from 1 to 10 carbon atoms. As used herein, “C₁-C₆ alkyl” may be selected from straight or branched chain hydrocarbyl groups containing from 1 to 6 carbon atoms. Representative examples are methyl, ethyl, n-propyl, iso-propyl, n-butyl, sec-butyl, iso-butyl, tert-butyl, n-pentyl, isopentyl, neopentyl, n-hexyl, isohexyl, neohexyl, etc. When a C₁-C₆ alkyl group is substituted, any hydrogen atom(s), CH₃, CH₂ or CH group(s) may be replaced with the substituent(s), providing valencies are satisfied.

As used herein, a protecting group may refer to any type of group or chemical moiety that can help prevent the atom to which it is attached, typically nitrogen or oxygen, from participating in undesired reactions. In particular, the use of protecting groups may be important to control reactions of a reactive group on a side chain or terminus of an amino acid during a peptide synthesis.

Suitable protecting groups may include side chain protecting groups and amino- or N-terminal protecting groups. In particular, where R¹ is a protecting group in formula (I) above, such may be considered as an N-terminal protecting group. In those examples where one or more of R², R³ and R⁵ is a protecting group, such may be considered as (a) side chain protecting group(s).

Protecting groups may be removed under different conditions. For example, a protecting group may be removed by base (e.g. base labile), acid (e.g. acid labile), removed by fluoride, removed by light (photolabile), or removed by hydrogenolysis.

The type of protecting group may be selected in accordance with the desired peptide synthetic strategy. Typically, side chain protecting groups will be selected which have an orthogonal reactivity to the N-terminal protecting groups. In other words, the selection of orthogonal protecting groups for the N-terminal and side chain protecting groups may allow the selective and/or specific deprotection of one of these types of groups without affecting the other.

Representative examples of suitable protecting groups include, but are not limited to, acyl-type protecting groups (such as formyl, acrylyl (Acr), benzoyl (Bz) and acetyl (Ac)); aromatic urethan-type protecting groups (such as benzyloxycarbonyl (Z) and substituted Z, such as p-chlorobenzyloxycarbonyl, p-nitrobenzyloxycarbonyl, p-bromobenzyloxycarbonyl, p-methoxybenzyloxycarbonyl); aliphatic urethan protecting groups (such as t-butyloxycarbonyl (BOC), diisopropylmethoxycarbonyl, isopropyloxycarbonyl, ethoxycarbonyl, allyloxycarbonyl); cycloalkyl urethan-type protecting groups (such as 9-fluorenyl-methyloxycarbonyl (Fmoc), cyclopentyloxycarbonyl, adamantyloxycarbonyl, and cyclohexyloxycarbonyl); thiourethan-type protecting groups (such as phenylthiocarbonyl); 2,2,4,6,7-pentamethyldihydrobenzofuran-5-sulfonyl (Pbf); tert butyl, triphenylmethyl (trityl); tetrahydropyranyl; benzyl ether (Bzl); 2,6-dichlorobenzyl (DCB); nitro, p-toluenesulfonyl (Tos); adamantyloxycarbonyl; xanthyl (Xan); benzyl; methyl; ethyl; -butyl ester, t-amyloxycarbonyl; photolabile groups (such as nitro, veratryl oxycarbonyl (NVOC)) and fluoride labile groups (such as trimethylsilylethyl oxycarbonyl (TEOC)).

In some examples, protecting groups include 9-fluorenylmethyloxycarbonyl (Fmoc) and 2,2,4,6,7-pentamethyldihydrobenzofuran-5-sulfonyl (Pbf), and t-butyloxycarbonyl (BOC).

In some examples, R¹ may be a protecting group labile under a first set of conditions and any one or more of R², R³ and R⁵ may each be independently selected from a protecting group labile under a second set of conditions. In other words, the protecting group of R¹ may have an orthogonal reactivity to the protecting group(s) of R², R³ and R⁵ (if there is protecting group present at these positions). In some examples, R¹ may be a base labile protecting group and any one or more of R², R³ and R⁵ may each be independently selected from a protecting group having an orthogonal reactivity (such as acid labile protecting groups). In some examples, R¹ may be Fmoc. In some examples, any one or more of R², R³ and R⁵ may each be independently selected from: Pbf and Boc. Modified amino acids of formula (I) that comprise protecting groups with these type of orthogonal reactivity may find particular application in Fmoc/tBu peptide synthesis strategies.

As used herein, the term “aryl” may be a single or fused ring system having one or more aromatic rings. The term “aryl” may refer to a mono- or polycyclic aromatic hydrocarbon system having 6 to 14 carbon ring atoms, in particular having 6 to 10 carbon ring atoms. Representative examples of suitable “aryl” groups include, but are not limited to, phenyl, biphenyl, naphthyl, 1-naphthyl, 2-naphthyl and anthracenyl. As used herein, “substituted aryl” refers to an aryl group as defined herein which comprises one or more substituents on the aromatic ring. When an aryl group is substituted, any hydrogen atom(s) may be replaced with the substituent(s), providing valencies are satisfied.

As used herein, “heteroaryl” may be a single or fused ring system having one or more aromatic rings containing 1 or more O, N and/or S heteroatoms. The term “heteroaryl” may refer to a mono- or polycyclic heteroaromatic system having 5 to 14 ring atoms, in particular having 5 to 10 ring atoms. Representative examples of heteroaryl groups may include, but are not limited to, pyrrolyl, furanyl, thiophenyl, pyrazolyl, imidazolyl, oxazolyl, isoxazolyl, thiazolyl, pyridinyl, pyrimidinyl, pyridazinyl, pyrazinyl, indolyl, benzofuranyl, benzothiazolyl, benzimidazolyl, indazolyl, benzoxazolyl, benzisoxazolyl etc. As used herein, “substituted heteroaryl” refers to a heteroaryl group as defined herein which comprises one or more substituents on the heteroaromatic ring. When a heteroaryl group is substituted, any hydrogen atom(s) may be replaced with the substituent(s), providing valencies are satisfied.

As used herein, the term “optionally substituted” means that the moiety may comprise one or more substituents.

As used herein, a “substituent” may include, but is not limited to, hydroxyl, thiol, carboxyl, cyano (CN), nitro (NO₂), halo, haloalkyl (e.g. a C₁ to C₆ haloalkyl), an alkyl group (e.g. C₁ to C₁₀ or C₁ to C₆), aryl (e.g. phenyl and substituted phenyl for example benzyl or benzoyl), alkoxy group (e.g. C₁ to C₆ alkoxy) or aryloxy (e.g. phenoxy and substituted phenoxy), thioether (e.g. C₁ to C₆ alkyl or aryl), keto (e.g. C₁ to C₆ keto), ester (e.g. C₁ to C₆ alkyl or aryl, which may be present as an oxyester or carbonylester on the substituted moiety), thioester (e.g. C₁ to C₆ alkyl or aryl), alkylene ester (such that attachment is on the alkylene group, rather than at the ester function which is optionally substituted with a C₁ to C₆ alkyl or aryl group), amine (including a five- or six-membered cyclic alkylene amine, further including a C₁ to C₆ alkyl amine or a C₁ to C₆ dialkyl amine which alkyl groups may be substituted with one or two hydroxyl groups), amido (e.g. which may be substituted with one or two C₁ to C₆ alkyl groups (including a carboxamide which is optionally substituted with one or two C₁ to C₆ alkyl groups), alkanol (e.g. C₁ to C₆ alkyl or aryl), or carboxylic acid (e.g. C₁ to C₆ alkyl or aryl), sulfoxide, sulfone, sulfonamide, and urethane (such as —O—C(O)—NR₂ or —N(R)—C(O)—O—R, wherein each R in this context is independently selected from C₁ to C₆ alkyl or aryl).

As used herein, “C₁-C₆ alkoxy” as used herein refers to a C₁-C₆ alkyl group, as defined above, appended to the parent molecular moiety through an oxy group, —O—. Representative examples of alkoxy include, but are not limited to, methoxy, ethoxy, propoxy, 2-propoxy, butoxy, tert-butoxy, pentyloxy, hexyloxy etc.

As used herein, “haloalkyl” may be an alkyl group in which one or more hydrogen atoms thereon have been replaced with a halogen atom. By way of a representative example, a C₁-C₆ haloalkyl may be a haloalkyl containing from 1 to 6 carbon atoms. The haloalkyl may be a fluoroalkyl, such as trifluoromethyl (—CF₃) or 1,1-difluoroethyl (—CH₂CHF₂). As used herein, a “halo” group may be F, Cl, Br, or I, typically F.

As used herein, a bicyclic ring system may refer to a chemical structure or moiety which comprises two rings joined together (e.g. which are covalently linked together). As used herein, a fused ring system may refer to a chemical structure or moiety that comprises two rings which share two adjacent atoms (or share one covalent bond). The bicyclic ring system may comprise from five to ten ring atoms. Representative examples include, but are not limited to, quinazolinyl, benzimidazolyl, tetrahydronaphthyridinyl (e.g. 1,2,3,4-tetrahydro-1,8-naphthyridinyl).

As used herein, the terms “aryl”, “substituted aryl”, “heteroaryl”, “substituted heteroaryl”, and “C₁-C₆ alkyl”, may refer to either a monovalent radical species or a divalent radical species. For example, within the context of the various formulae described herein, R³ is typically a monovalent group that is attached to the parent structure and so the term C₁-C₆ alkyl should be understood to represent a monovalent radical moiety. By way of further example, X (as shown in formula (I) is typically a divalent group that is covalently attached to both the carbon atom at the starred (*) position and the A group. As such, in these examples, the term “C₁-C₆ alkyl” should be understood to represent a divalent radical moiety. Similar considerations apply to B (as shown in formula (I) which again is typically a divalent group that is covalently attached to both X and Y (where present). As such, in these examples and with reference to group B, the terms “aryl”, “heteroaryl” and “C₁-C₆ alkyl” should each be understood to represent a divalent radical moiety.

As stated above, R¹ may be selected from H and protecting group. In some examples, R¹ may be a base labile protecting group. Representative examples include, but are not limited to, 9-Fluorenylmethyloxycarbonyl (Fmoc), benzoyl (Bz), acetyl (Ac) and the like. In some examples, R¹ may be H or Fmoc.

As stated above, X may be absent. In those examples where X is absent, A is directly attached to the starred carbon atom shown in formula (I) (e.g. by way of a covalent bond).

In other examples, X may be selected from optionally substituted C₁-C₆ alkyl, optionally substituted C₁-C₆ alkyl-NR²(C═O)—, and optionally substituted —C₁-C₆ alkyl-(C═O)NR². In other examples, X may be selected from optionally substituted C₁-C₃ alkyl, optionally substituted C₁-C₃alkyl-NR²(C═O)—, and optionally substituted —C₁-C₃alkyl-(C═O)NR². In some examples, X may be absent or may be selected from —CH₂— and —CH₂NR²(C═O)— (e.g. —CH₂NH(C═O)—), and —CH₂(C═O)NR²— (e.g. —CH₂(C═O)NH—).

As stated above, A may be:

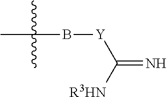

B may be selected from optionally substituted aryl, optionally substituted heteroaryl and optionally substituted C₁-C₆ alkyl (such as optionally substituted C₁-C₃ alkyl).

Where B is selected from optionally substituted aryl and optionally substituted heteroaryl, it will be appreciated that the groups X (as shown on formula (I)) and Y (as shown above) may be appended to the aromatic ring (e.g. by way of covalent bonds) at any position and in any substitution pattern. For example, the groups may be appended to the aryl or heteroaryl ring in an ortho, meta or para-substitution pattern in relation to one another. These moieties X and Y may be appended to the aryl or heteroaryl ring by way of a covalent bond to a carbon atom present on the aryl or heteroaryl ring.

In some examples, B may be a 6- to 10-membered aryl ring, which is optionally substituted with from one to three substituents each independently selected from halo, C₁-C₆ alkyl, C₁-C₆ haloalkyl and C₁-C₆ alkoxy.

In some examples, B may be optionally substituted phenyl. For example, B may be phenyl. In other examples, B may be a halo-substituted phenyl comprising one or more halo substitutions (e.g. fluoro, chloro, bromo, iodo and the like). In some examples, B may a difluoro-substituted phenyl group.

In some examples, B may be 5- to 10-membered heteroaryl ring, containing from one to three heteroatoms selected from N, O and S, and being optionally substituted with from one to three substituents each independently selected from halo, C₁-C₆ alkyl, C₁-C₆ haloalkyl and C₁-C₆ alkoxy.

In some examples, B may be optionally substituted pyridyl.

Where B is an optionally substituted C₁-C₆ alkyl, Y is absent. In such examples, B may be an ethyl group.

As stated above, R³ may be selected from H, optionally substituted C₁-C₆ alkyl and protecting group. In some examples, R³ may be C₁-C₆ alkyl (e.g. methyl). In other examples R³ may be a protecting group such as an acid labile protecting group (e.g. 2,2,4,6,7-pentamethyl-dihydro-benzofuran-5-sulfonyl (Pbf).

As stated previously, A may be or comprise a bicyclic fused ring system. The bicyclic ring system may comprise an amidine-like motif according to the following formula:

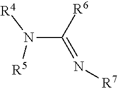

wherein R⁴ and R⁶ are each independently selected from H and a carbon atom that forms part of a backbone of the bicyclic ring system;
R⁵ is selected from H and protecting group; and
R⁷ is a carbon atom that forms part of a backbone of the bicyclic ring system.

In some examples, the bicyclic ring may be a heterocyclic ring comprising one or more heteroatoms (e.g. N atoms). In some examples, the bicyclic ring may be a nine- or ten-membered ring system.

In some examples, the two N atoms of the amidine-like motif shown above also form part of the backbone of the bicyclic ring system. In other examples, only the N atom directly attached to the R⁷ group forms part of the backbone of the bicyclic ring system (and the NR⁴R⁵ group in such examples is a substituent on the bicyclic ring system).

In some examples, the bicyclic ring may comprise at least one aromatic or heteroaromatic ring. The bicyclic ring may be selected from quinazolinyl, benzimidazolyl, and tetrahydronaphthyridinyl (e.g. 1,2,3,4-tetrahydro-1,8-naphthyridinyl).

Representative examples of suitable A groups are shown below:

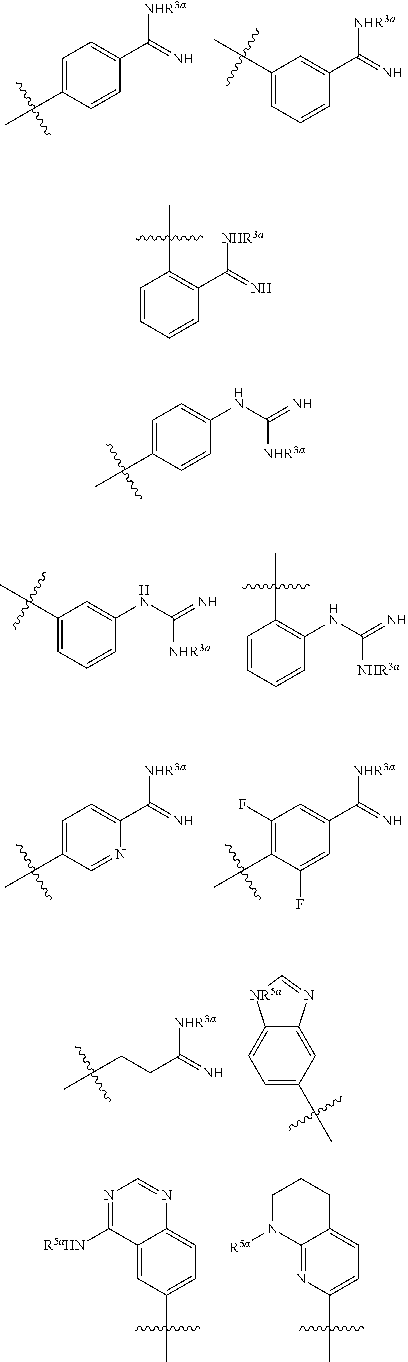

In each of the structures shown above, R^{3a} may be selected from H, C₁ to C₆ alkyl (e.g. methyl) and acid labile protecting group (e.g. 2,2,4,6,7-pentamethyl-dihydro-benzofuran-5-sulfonyl (Pbf) or Boc); and/or
R^{5a} may be selected from H, C₁ to C₆ alkyl (e.g. methyl) and acid labile protecting group (e.g. 2,2,4,6,7-pentamethyl-dihydro-benzofuran-5-sulfonyl (Pbf) or Boc).

In each of the structures above, the bond intersected by the wavy line indicates the point of attachment of the structure to the group X shown on formula (I).

In some examples, the modified amino acid may be represented by formula (Ia):

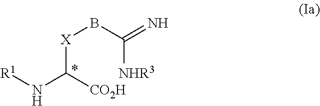

wherein
   R¹ is H or a protecting group;
   X is selected from optionally substituted C₁-C₆ alkyl, optionally substituted C₁-C₆ alkyl-NR²(C═O)— and optionally substituted —C₁-C₆ alkyl-(C═O)NR²—;
   R² is selected from H, optionally substituted C₁-C₆ alkyl and protecting group;
   B is selected from optionally substituted aryl and optionally substituted heteroaryl; and
   R³ is selected from H, optionally substituted C₁-C₆ alkyl and protecting group.

By way of further example, R¹, X, R², B and R³ of formula (Ia) are further defined as described herein and above with respect to formula (I) (unless the context indicates otherwise).

A representative example of a modified amino acid in accordance with the present disclosure is shown as formula (Ib) below:

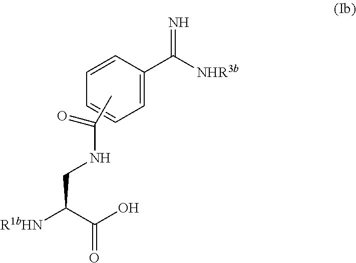

wherein R^{1b} is selected from H and protecting group (e.g. Fmoc); and
R^{3b} is selected from H, C₁-C₆ alkyl and protecting group (e.g. Pbf).

In this example, the two groups may be appended to the aryl ring in any substitution pattern, e.g. ortho-, meta-, or para-substitution pattern in relation to one another. In particular the pendant group may be covalently bonded to a carbon atom on the aryl ring at any chemically suitable position (e.g. by replacing a hydrogen atom).

Further representative examples of a modified amino acid in accordance with the present disclosure are shown below:

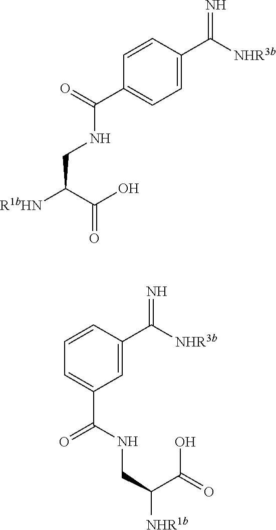

wherein R^{1b} is selected from H or protection group (e.g. Fmoc); and
R^{3b} is selected from H, C₁-C₆ alkyl and protecting group (e.g. Pbf).

A yet further representative example of a modified amino acid in accordance with the present disclosure is shown as formula (Ic) below

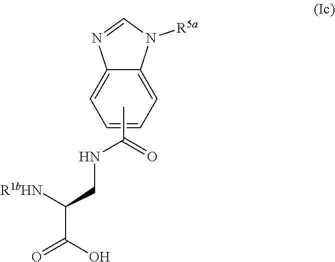

wherein R^{1b} is selected from H or protecting group (e.g. Fmoc); and
R^{5a} is selected from H, C₁-C₆ alkyl and protecting group (e.g. Pbf or Boc).

In this example, the group comprising the amino acid moiety may be appended to the benzimidazole core at any suitable position by way of a covalent bond to a carbon atom on the heteroaryl ring (e.g. by replacing a hydrogen atom at that position), e.g. at the 4, 5, 6 or 7 position. In some examples, the modified amino acid may comprise one of the following structures:

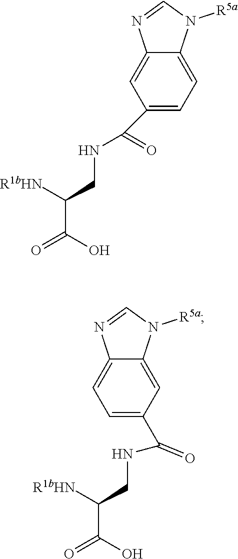

wherein R^{1b} and R^{5a} are as defined above.

In some examples of the above, where R^{5a} is H, the two structures shown above may exist as a mixture (as they represent tautomeric forms of one another). In some cases, where R^{5a} represents a protecting group, the synthesis of the modified amino acid may provide the two structures shown above as a mixture. The mixture may be used to synthesis a cell penetrating peptide. Following removal of protecting group (e.g. after the cell penetrating peptide has been synthesised), R^{5a} is H, and the two structures can readily convert via tautomerism.

Further representative examples of modified amino acids in accordance with the present disclosure are shown below:

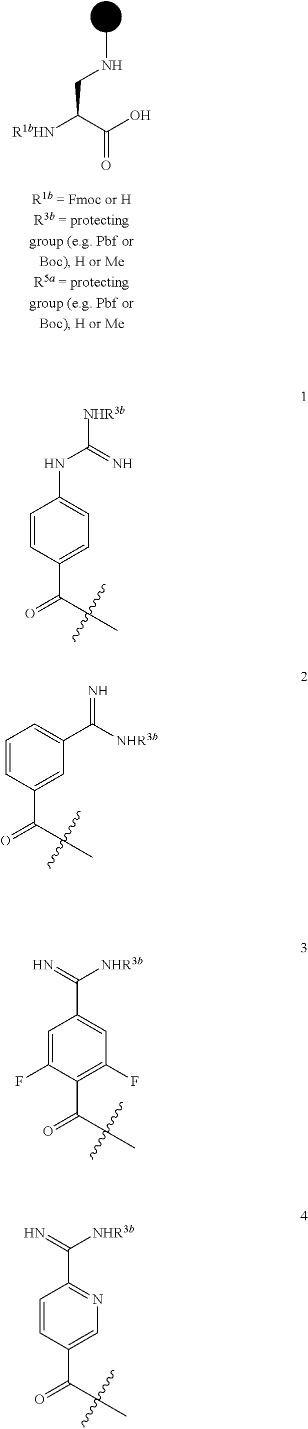

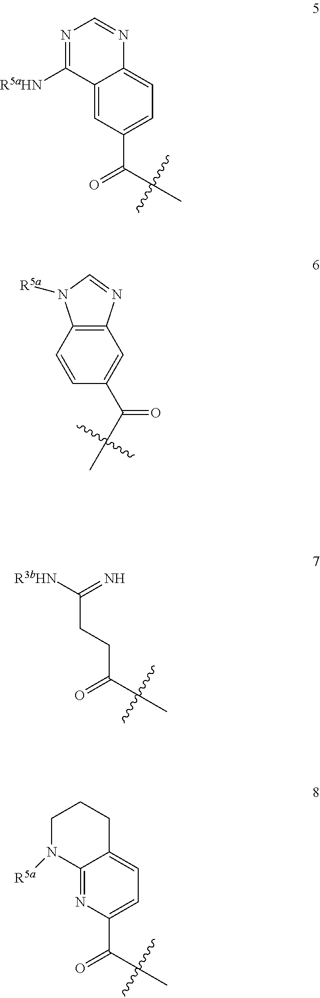

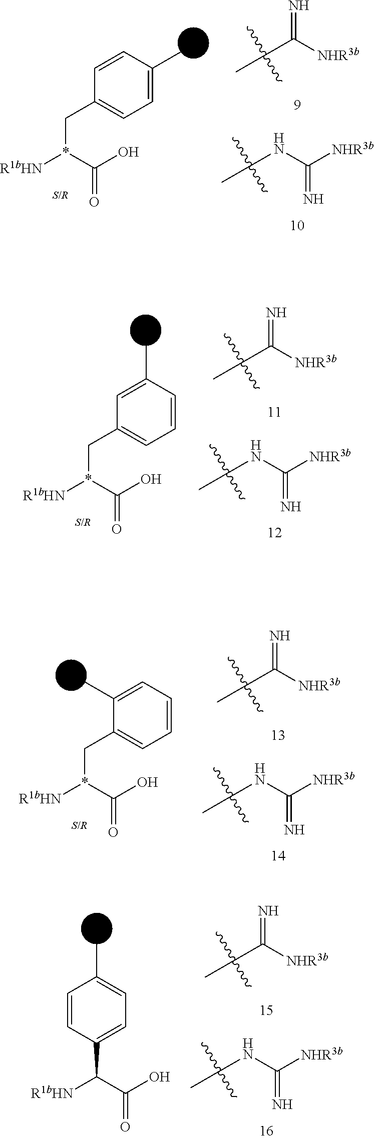

In each of the compounds above, R^{1b} is Fmoc or H; and R^{3b} is protecting group (e.g. Pbf or Boc), H or methyl.

The modified amino acids as described above may be used to provide peptides, and may be particularly useful in providing cell penetrating peptides in which they can be used to replace arginine. Accordingly, there is further provided a peptide, particularly a cell penetrating peptide, comprising a residue derived from a modified amino acid as described herein. As such, in the various formulae and structures noted above, it will be appreciated that an amino acid residue may be present at R¹ (in place of H and protecting group) and/or an amino acid residue may be present in place of H at the carboxylic acid group. In particular, once incorporated into the cell penetrating peptide, there will be at least one amino acid residue present at either R¹ or in place of the H of the carboxylic acid group. In some examples, there may be an amino acid residue present at R¹ and an amino acid residue present in place of the H of the carboxylic acid group. Whilst the various formulae and structures for the at least one modified amino acid are given in the context of an isolated amino acid, it will be appreciated that these are equally applicable to a cell penetrating peptide comprising a residue derived from the modified amino acid. By way of example, the various options for (A) and (X) as given for the modified amino acid of formula (I) above, are equally applicable to a cell penetrating peptide comprising a residue derived from this modified amino acid.

By way of example, there is provided a peptide, particularly a cell penetrating peptide, comprising at least one modified amino acid residue according the formula (II):

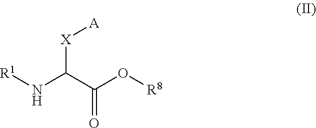

wherein R¹ is selected from H, protecting group and an amino acid residue;
R⁸ is selected from H and an amino acid residue; and
X and A are as defined herein and above for formulae (I) and (Ia).

The at least one modified amino acid residue may not be derived from a modified amino comprising the following structure:

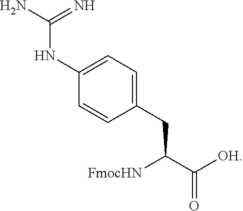

In such examples, the cell penetrating peptide may comprise the at least one modified amino acid together with one additional amino acid residue at either R¹ or R⁸.

In yet further examples, the cell penetrating peptide may comprise the at least one modified amino acid together with two additional amino acid residues, one at each of R¹ and R⁸.

By way of further example, the peptide may comprise a modified amino acid residue according to formula (IIa) or (IIb):

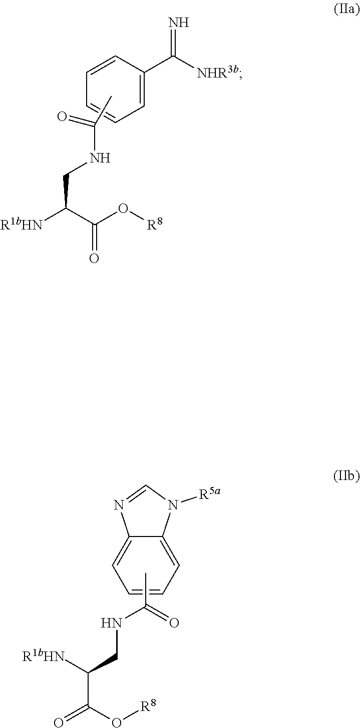

wherein R^{1b} is selected from H, protecting group (e.g. Fmoc) or amino acid residue;
R⁸ is selected from H and an amino acid residue; and
R^{3b} and R^{5a} are as defined above and herein (e.g. as for formula (Ib) and (Ic).

In some examples, R¹ and R⁸ may each be independently selected from H and an amino acid residue. In some examples, at least one of R¹ and R⁸ is an amino acid residue.

The peptide (e.g. the cell penetrating peptide) may comprise any number of amino acid residues e.g. between about 2 and 100 amino acid residues, such as between about 5 and 50 or about 7 and 20 amino acid residues. The cell penetrating peptide may comprise between about 2 and 30 amino acid residues.

The peptide (e.g. the cell penetrating peptide) may comprise a plurality of modified amino acid residues falling under the scope of formula (II), (IIa), or (IIb), or derived from a modified amino acid as defined or described in relation to any one of formula (I), (Ia), (Ib) or (Ic). By way of example, the peptide may comprise at least 2, 3, 4, 5, 6, 7, 8, 9, or 10 amino acid residues falling under the scope of formula (II), (IIa) or (IIb) or derived from a modified amino acid as defined or described in relation to any one of formula (I), (Ia), (Ib) or (Ic). In some cases, at least about 10%, at least about 20%, at least about 30%, at least about 40% or at least about 50% of the amino acid residues present in the peptide may be one or more amino acid residues according to formula (II) or derived from a modified amino acid as defined or described in relation to any one of formula (I), (Ia), (Ib) or (Ic).

The peptide may be a cell penetrating peptide, such as a cationic cell penetrating peptide (those cell penetrating peptides carrying a net positive charge). As stated previously, cell penetrating peptides generally comprise relatively high numbers and/or proportions of arginine residues. In such peptides, one or more arginine residues may be replaced by an amino acid residue according to formula (II), (IIa), or (IIb), or derived from a modified amino acid as defined or described in relation to any one of formula (I), (Ia), (Ib) or (Ic). Indeed, as explained previously, the modified amino acids disclosed herein may find particular utility in cationic CPPs as they can be used to provide an overall positive charge whilst showing a modulated basicity and/or pKa. For example, where an amidino group replaces a guanidino group (as found in an arginine residue) (or a different bridging group is used between the alpha carbon of the amino acid and a guanidino or amidino group of the modified amino acid), the same overall positive charge may result, but the distribution of that charge throughout the structure may be different, providing a modulated basicity and/or pKa.

In some examples, the cell penetrating peptide may comprise a c Log P value of between about −42.12 and about 2.97 (see, for example, Oliveira et al, Nature Scientific Reports, 2021, 11, 7628).

In some examples, the cell penetrating peptide may comprise an overall formal charge between about +1 and about +4 (such as about +2 or +3). In some examples, the cell penetrating peptide may comprise an overall formal charge of about +2. Indeed, it has been observed that to facilitate cellular entry of a peptide, a peptide may preferably hold a positive charge, otherwise there may be little to no cellular incorporation using fluorescent detection methods. This phenomenon has generally been explored in the context of stapled peptides (see, for example, Verdine et al, Med. Chem. Commun., 2015, 6, 111-119), in which a number of stapled peptides were tested with various overall charges for cellular penetration), but, without being bound by theory, the inventors hypothesise that a similar pattern may exist for cell penetrating peptides.

The cell penetrating peptide may comprise any suitable membrane diffusion rate and/or rate of cellular uptake that allows the cell penetrating peptide to cross a membrane and/or carry a cargo into a cell at a suitable rate (which may depend on the cargo and/or purpose of the cargo). In some examples, a suitable membrane diffusion rate may be about Pₐₚₚ (apparent permeability) greater than or equal to 1×10⁻⁶ cm/s (e.g. in a Caco permeability assay or a parallel artificial membrane permeability assay (PAMPA)). (See, for example, Pei et al, Chem. Rev. 2019, 119, 10241-10287).

In some examples, the cell penetrating peptide may comprise a hydrophobic moment of at least about 0.30, at least about 0.40, or at least about 0.50. In some examples the cell penetrating peptide may comprise a hydrophobic moment of about 0.55. (See, for example, Bird et al, Nature Chemical Biology, Vol. 12, October 2016, 845 which describes an investigation of stapled peptides and shows a linear relationship between hydrophobic moment and cellular access, data appeared to show a linear progression without any top-end value identified. Without being bound by theory, the inventors hypothesise that a similar pattern may exist for cell penetrating peptides).

Examples of cell penetrating peptide sequences are shown in FIG. 1b. In particular, an example of a cell penetrating peptide is Tat (Green, M.; Ishino, M.; Loewenstein, P. M. Mutational analysis of HIV-1 Tat minimal domain peptides: Identification of <em>trans</em>-dominant mutants that suppress HIV-LTR-driven gene expression. Cell 1989, 58, 215-223). The sequence of Tat is shown as SEQ ID NO: 1 below. Further examples include TP-1 (SEQ ID NO: 2), TP-2 (SEQ ID NO: 3) and PiP6 (SEQ ID NO: 4) (Guha et al, Mechanistic Landscape of Membrane-Permeabilizing Peptides. Chem. Rev. 2019, 119, 6040-6085; Marks et al, Spontaneous Membrane-Translocating Peptides by Orthogonal High-Throughput Screening. J. Am. Chem. Soc. 2011, 133, 8995-9004; and Betts et al, Pip6-PMO, A New Generation of Peptide-oligonucleotide Conjugates With Improved Cardiac Exon Skipping Activity for DMD Treatment. Mol. Ther. Nucleic Acids 2012, 1).

SEQ ID NO: 1
RKKRRQRRR-
SEQ ID NO: 2
PLILLRLLRG-
SEQ ID NO: 3
PLIYLRLLRG-
SEQ ID NO: 4
RXRRBRRYQFLIRBRXR-

In SEQ ID NO: 4 above, X in the amino acid sequence represents 6-aminohexanoic acid (Ahx) and B in the amino acid sequence represents p-alanine.

By way of example only, an amino acid residue of formula (II) (e.g. formula (IIa) or (IIb)), or an amino acid residue derived from a modified amino acid as defined or described in relation to any one of formula (I), (Ia), (Ib) or (Ic), may be used in place of one or more arginine residues in any one or more of the sequences described above.

By way of further example, a cell penetrating peptide comprising an amino acid residue according to formula (II) is shown below as SEQ ID NO: 5.

SEQ ID NO: 5
PLIYLXLLXG-

Wherein X in the amino acid sequence is an amino acid residue according to formula (II), or is a residue derived from a modified amino acid as defined or described in relation to any one of formula (I), (Ia), (Ib) or (Ic).

In some examples, X in the amino acid sequence may be an amino acid residue according to formula (IIa) or (IIb).

Additional examples of cell penetrating peptides in which one or arginine residues may be replaced with an amino acid residue of formula (II), or with an amino residue derived from a modified amino acid as defined or described in relation to any one of formula (I), (Ia), (Ib) or (Ic), are shown below:

(“R9”)
SEQ ID NO: 6
RRRRRRRRR-
(Penetratin)
SEQ ID NO: 7
RQIKIWFQNRRMKWKK-
(ARF(1-22)
SEQ ID NO: 8
MVRRFLVTLRIRRACGPPRVRV-

In some examples, the cell penetrating peptide may comprise a sequence as set out in SEQ ID NO: 9:

SEQ ID NO: 9
XPLIYLAmLLAmG-

wherein X in the sequence above (SEQ ID NO: 9) is a residue derived from 6-aminohexanoic acid (Ahx) and each Am in the sequence above is a modified amino acid residue according to the following structure:

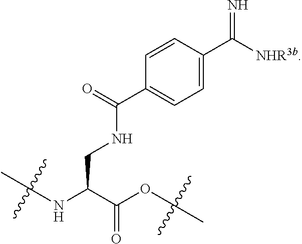

In the above structure, the wavy line intersects the peptide bond that forms between the Am residue and the neighbouring amino acids.

In some examples, the cell penetrating peptide may comprise a sequence as set out in SEQ ID NO: 10:

SEQ ID NO: 10
XPLIYLBimLLBimG-

wherein X in the sequence above (SEQ ID NO: 10) is a residue derived from 6-aminohexanoic acid (Ahx) and each Bim is a modified amino acid residue according to the following structure:

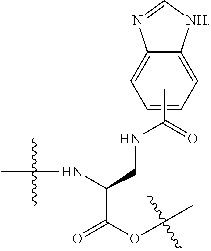

In the above structure, the wavy line intersects the peptide bond that forms between the Bim residue and the neighbouring amino acids. As explained previously, the Bim modified amino acid may exist in different tautomeric forms in the cell penetrating peptide structure, which may readily interconvert:

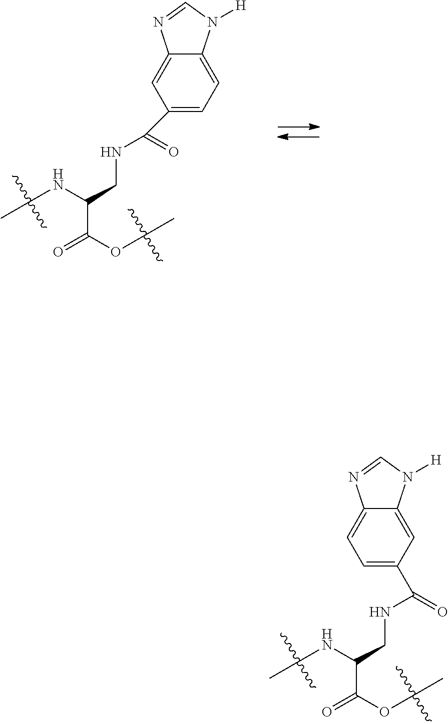

In some examples, the cell penetrating peptide may comprise a sequence as set out in SEQ ID NO: 11:

SEQ ID NO: 11
XPLIYLmAmLLmAmG-

wherein X in the sequence above (SEQ ID NO: 11) is a residue derived from 6-aminohexanoic acid (Ahx) and each mAm is a modified amino acid residue according to the following structure:

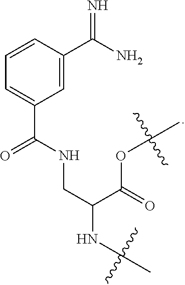 [SMILES: *NC(CNC(=O)c1cccc(C(=N)N)c1)C(=O)O*]

In the above structure, the wavy line intersects the peptide bond that forms between the mAm residue and the neighbouring amino acids.

The cell penetrating peptide may further comprise an agent of interest. The agent of interest may be associated with the peptide either via a chemical linkage (e.g. covalent bond) and/or via non-covalent interactions using methods known in the art.

As stated previously, the agent of interest may be any type of entity that requires delivery into and/or distribution throughout a cell. The agent of interest may be for example, a therapeutic agent, a diagnostic agent or a contrast agent. The agent of interest may be a biological molecule (e.g. a nucleic acid-based molecule (e.g. siRNA, antisense oligonucleotide, DNA, plasmid, etc.), a polypeptide or protein). In some examples, the agent of interest may be a particle (e.g. nanosize particle) or a chemical compound.

In some examples, the agent of interest may be a fluorescent tag (such as fluorescein or a fluorescein derivative such as fluorescein isothiocyanate (FITC)). In some examples, the fluorescent tag may be covalently linked to the peptide at any chemically suitable position.

Where the agent of interest is a therapeutic agent, the cell penetrating peptide may be useful to facilitate the cellular uptake and/or distribution of the agent.

Accordingly, there is further provided a cell penetrating peptide as described herein for use in therapy and/or medicine.

Indeed, the disclosure also encompasses a method of treatment comprising administering a cell penetrating peptide as described herein (e.g. a cell penetrating peptide comprising a therapeutic agent) to a subject in need thereof. The cell penetrating peptide may be administered in a therapeutically effective amount.

There is also provided use of a cell penetrating peptide as described herein (e.g. a cell penetrating peptide comprising a therapeutic agent) in the manufacture of a medicament for use in therapy and/or medicine.

Where a cell penetrating peptide is for use in therapy and/or medicine (e.g. a cell penetrating peptide comprising a therapeutic agent), then it may be formulated in a pharmaceutical composition. For example, the cell penetrating peptides of this disclosure may be formulated as sterile pharmaceutical compositions suitable for administration to subjects.

Such formulations may comprise one or more pharmaceutically acceptable excipients, carriers and/or diluents. Representative examples include, but are not limited to, water, saline, phosphate buffered saline, dextrose, glycerol, ethanol, ion exchangers, alumina, aluminium stearate, lecithin, serum proteins, such as serum albumin, buffer substances such as phosphates, glycine, sorbic acid, potassium sorbate, partial glyceride mixtures of saturated vegetable fatty acids, water salts or electrolytes, such as protamine sulphate, disodium hydrogen phosphate, potassium hydrogen phosphate, sodium chloride, zinc salts, colloidal silica, magnesium trisilicate, polyvinyl pyrrolidone, cellulose-based substances, polyethylene glycon, sodium carboxymethylcellulose, polyacrylates, waxes, polyethylene-polypropylene-block polymers, polyethylene glycol and wool fat and the like, or combinations thereof.

The pharmaceutical compositions may be formulated and/or prepared for, for example, oral, parenteral, topical and/or mucosal/inhalation administration.

According to a further aspect, there is provided the use of a modified amino acid as described herein (e.g. as per formula (I), (Ia), (Ib), or (Ic)) in the synthesis of a peptide. In particular, there is provided a method of preparing a peptide (e.g. a cell penetrating peptide) comprising the use of a modified amino acid according to formula (I), (Ia), (Ib), or (Ic).

In particular, the method may comprise contacting a first amino acid (or peptide fragment comprising a residue of the first amino acid) with a second amino acid (or peptide fragment comprising a residue of the second amino acid) under such conditions so as to promote and/or facilitate a condensation reaction between the first and second amino acids (or peptide fragments comprising residues of the same) to provide a new peptide link.

At least one of the first and second amino acids may be a modified amino acid as described herein (e.g. a modified amino acid according to formula (I), (Ia), (Ib), or (Ic)).

Thus, the method provides a peptide comprising at least one modified amino acid residue according to formula (II).

As will be understood by the skilled person, the peptide may be prepared using a chemical synthetic approach e.g. by way of a solid phase or liquid phase peptide synthesis. Such chemical synthetic approaches to peptides generally involve a number of coupling (e.g. condensation) reactions between amino acids.

The method may comprise a series of (a) deprotection and (b) coupling steps that are repeated until a desired or target peptide is obtained. Following each series of deprotection and coupling steps, an amino acid may be added to a growing peptide fragment. In this way, the synthesis of the peptide may be controlled by way of a sequential addition of amino acids.

In particular, orthogonal protecting group strategies may be employed in such syntheses (e.g. where different classes of protecting group may be used to protect the N-terminus of the amino acid to those used to protect reactive groups on side chains of the amino acids). Such strategies may be employed to ensure side reactions are minimised during peptide synthesis and are generally known in the art. Representative examples include, but are not limited to, a Boc/Bzl protecting group strategy (e.g. where N-termini of amino acids are protected with an acid labile Boc group and side chain protecting groups are benzyl or benzyl-based groups) and a Fmoc/tBu or Fmoc/Boc protecting group strategy (e.g. where N-termini of amino acids are protected with a base labile Fmoc group and side chain protecting groups are acid labile groups (e.g. tBu or Boc).

The deprotection step (a) may comprise removing a protecting group from a terminus of the amino acid (typically an N-terminus). By way of example only, the deprotection step may comprise removing a protecting group from the N-terminus of an amino acid (or peptide fragment comprising at least one amino acid residue). In some examples, the deprotection step may comprise removing a base labile protecting group (such as Fmoc) from the N-terminus of an amino acid (or peptide fragment comprising at least one amino acid residue).

The coupling step (b) may comprise contacting a first amino acid (or peptide fragment comprising at least one amino acid residue) with a second amino acid under such conditions so as to promote and/or facilitate a condensation reaction between the N-terminus of the first amino acid (or peptide fragment) and the C-terminus of the second amino acid to provide a peptide or fragment thereof. The second amino acid may comprise a protecting group on the N-terminus that may be stable under the coupling conditions (and so prevent or reduce unwanted condensation reactions).

In order to facilitate and/or promote the coupling step, activating agents and/or catalysts may be added to increase the reactivity of the N-terminus acid and/or the C-terminus of the first amino acid and/or the second amino acid.

Suitable activating agents are known to those skilled in the art. Representative examples may include but, are not limited to, carbodiimide-based reagents (e.g. dicyclohexylcarbodiimide (DCC) and diisopropylcarbodiimide (DIC)), 1-hydroxy-benzotriazole (HOBt), and 1-hydroxy-7-aza-benzotriazole (HOAt), aminium, uronium and/or phosphonium salts (e.g. HATU (1-[Bis(dimethylamino)methylene]-1H-1,2,3-triazolo[4,5-b]pyridinium 3-oxide hexafluorophosphate, HBTU (2-(1H-benzotriazol-1-yl)-1,1,3,3-tetramethyluronium hexafluorophosphate, PyBOP (benzotriazol-1-yloxytripyrrolidinophosphonium hexafluorophosphate), and the like. In some examples, an amino acid may be converted into an acid halide (e.g. an acyl fluoride or acyl chloride) to promote the condensation step.

In some examples, the method may further comprise a step of deprotecting one or more or all of the side chain protecting groups (if present). The step of deprotecting one or more or all of the side chain protecting groups may take place after a desired peptide length or sequence has been obtained and/or after cleavage of the peptide from a solid support.

Where a solid support is used, the method may comprise providing a first amino acid which may optionally be linked (e.g. covalently linked) to a solid support. In such examples, the peptide may be cleaved from the support when a desired peptide length or sequence has been obtained.

When a solid phase synthesis is employed, any type of support suitable in the practice of solid phase peptide synthesis (SPPS) can be used. By way of example, the support may comprise a resin that can be made from one or more polymers, copolymers, or combinations of polymers such as polyamide, polysulfamide, substituted polyethylenes, polyethylene glycol, phenolic resins, polysaccharides, or polystyrene. The solid support typically includes a linking moiety to which the growing peptide is coupled during synthesis and which can be cleaved under desired conditions to release the peptide from the support.

Suitable solid supports can include linkers that are photocleavable, trifluoroacetic acid-cleavable (TFA-cleavable), HF-cleavable, fluoride ion-cleavable, reductively-cleavable, Pd(O)-cleavable, nucleophilically-cleavable, or radically-cleavable. In some examples, the linking moieties may be cleavable under conditions such that any side chain protecting groups are stable and/or are not removed under the conditions used to cleave the linker.

In some examples, solid supports may include acid sensitive solid supports, for example, Rink amide resins, hydroxymethyl-polystyrene-divinylbenzene polymer resin (“Wang” resins, see Wang, S. S. 1973, J. Am. Chem. Soc., 95:1328-33), 2-chlorotrityl chloride resin (see Barlos et al. (1989) Tetrahedron Letters 30(30):3943-3946), and 4-hydroxymethyl-3-methoxyphenoxybutyric acid resin (see Richter et al. (1994), Tetrahedron Letters 35(27):4705-4706), as well as functionalized, crosslinked poly N-acryloylpyrrolidone resins, and chloromethylpolystyrene dinvinylbenzene polymer resins.

A yet further aspect of the disclosure is directed to a method for screening for a cell penetrating peptide.

The method may comprise providing a candidate peptide which comprises at least one modified amino acid residue in accordance with formula (II), or comprises at least one modified amino acid residue derived from the modified amino acid of formula (I), (Ia), (Ib), or (Ic).

The method may comprise contacting the candidate peptide with a cell. The method may comprise determining an effect of the candidate peptide on the cell. The method may be an in vitro method.

By way of example only, the method may comprise determining one or more of:
(i) a cellular uptake of the candidate peptide;
(ii) a cellular distribution of the candidate peptide; and/or
(iii) a toxicity of the candidate peptide (on the cell).

Where a candidate peptide is determined to have been taken up into the cell and/or distributed throughout the cell, the candidate peptide may be determined to be suitable for use as a cell penetrating peptide. Additionally or alternatively, where a candidate peptide shows minimal or low levels of toxicity, the candidate peptide may be determined to be suitable for use as a cell penetrating peptide.

In some examples, the candidate peptide may be labelled (e.g. fluorescently labelled). Such labelling may assist in the determination and/or detection of the effects of the candidate peptide on the cell.

In some examples, the method may comprise identifying a peptide sequence that is able to function as a cell penetrating peptide (e.g. a peptide sequence known to act as a cell penetrating peptide). Such a peptide may be designated as the parent peptide and may comprise at least one arginine residue.

The method may comprise replacing one or more arginine residues in the parent peptide with a modified amino acid residue in accordance with formula (II). Such a modified peptide may be referred to as the candidate peptide.

The method may comprise comparing an effect of the candidate peptide on the cell to a reference level. The reference level may be obtained by determining an effect of the parent peptide on the cell.

By way of example only, the method may comprise determining one or more of:
(i) a cellular uptake of the parent peptide;
(ii) a cellular distribution of the parent peptide; and/or
(iii) a toxicity of the parent peptide (on the cell);
   to provide the reference level.

In those cases where an effect of the candidate peptide is more favourable (e.g. to facilitate use as a cell penetrating peptide) than a reference level observed with the parent peptide, then the candidate peptide may provide a useful and/or improved cell penetrating peptide and/or the modified amino acid may find particular utility in a cell penetrating peptide.

By way of example only, an effect of the candidate peptide may be considered more favourable where the candidate peptide shows an increased cellular uptake and/or cellular distribution in comparison to the parent peptide. In some examples, an effect of the candidate peptide may be considered more favourable where the candidate peptide shows a decreased level of toxicity in comparison to the parent peptide.

The inventors have further identified a series of novel compounds, which may, for example, find particular application in the synthesis of cell penetrating peptides. In particular, according to a further aspect of the disclosure, there is provided a modified amino acid according to formula (I″):

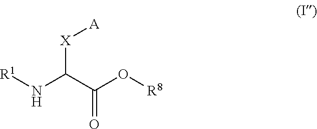

wherein:
R¹ is H or protecting group (e.g. Fmoc);
R⁸ is H or protecting group (C₁-C₆ alkyl); and
(i) X is selected from optionally substituted C₂-C₄ alkyl, optionally substituted —C₁-C₃ alkyl-NR²(C═O)— and optionally substituted C₁-C₃ alkyl-(C═O)NR²—;
R² is selected from H, optionally substituted C₁-C₆ alkyl and protecting group; and A is:

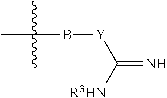

wherein B is selected from optionally substituted aryl, optionally substituted heteroaryl and optionally substituted C₁-C₆ alkyl (such as C₁-C₃ alkyl);
Y is absent;
R³ is selected from H, optionally substituted C₁-C₆ alkyl and protecting group;
or
(ii) X is absent, or is selected from optionally substituted C₁-C₆ alkyl (e.g. C₁-C₃ alkyl), optionally substituted —C₁-C₆ alkyl-NR²(C═O)— (e.g. —C₁-C₃ alkyl-NR²(C═O)—), and optionally substituted C₁-C₆ alkyl-(C═O)NR²— (e.g. C₁-C₃ alkyl-(C═O)NR²—);
R² is selected from H, optionally substituted C₁-C₆ alkyl and protecting group; and
A is selected from:

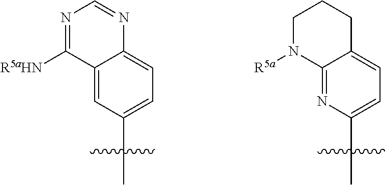

wherein R^{5a} is selected from H and protecting group.

In some examples of formula (I″), R¹ may be H or 9-fluorenylmethyloxycarbonyl (Fmoc).

In some examples of formula (I″), X is —CH₂NH(C═O)—. In some examples of formula (I″), A is:

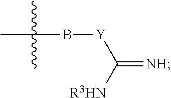

wherein:
B is optionally substituted phenyl or optionally substituted pyridyl;
Y is absent; and
R³ is optionally substituted C₁-C₆ alkyl or an acid labile protecting group.

In some examples, the modified amino acid may be represented by formula (Ia″):

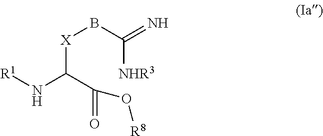

wherein
R¹ is H or a protecting group;
R⁸ is H or a protecting group;
X is selected from optionally substituted C₂-C₄ alkyl, optionally substituted —C₁-C₃ alkyl-NR²(C═O)— and optionally substituted C₁-C₃ alkyl-(C═O)NR²—;
R² is selected from H, optionally substituted C₁-C₆ alkyl and protecting group;
B is selected from optionally substituted aryl and optionally substituted heteroaryl; and
R³ is selected from H, optionally substituted C₁-C₆ alkyl and protecting group.

In preferred examples, R¹ is protecting group and R⁸ is H.

In some examples of formula (I″), A is selected from:

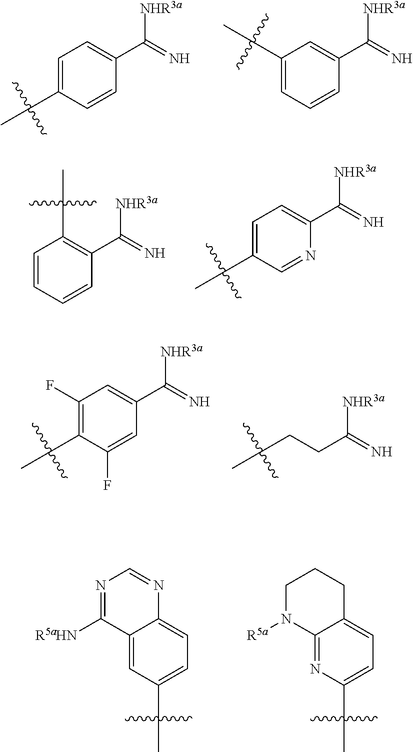

wherein R^{3a} is selected from H, C₁ to C₆ alkyl (e.g. methyl) and acid labile protecting group (e.g. 2,2,4,6,7-pentamethyl-dihydro-benzofuran-5-sulfonyl (Pbf) or Boc); and
R^{5a} is selected from H, C₁ to C₆ alkyl (e.g. methyl) and acid labile protecting group (e.g. Pbf or Boc).

In some examples, the modified amino acid may comprises a structure according to formula (Ib″):

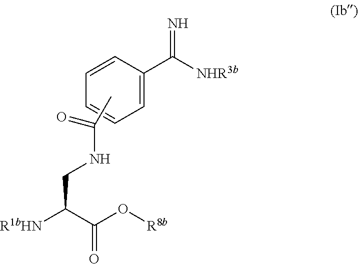

wherein R^{1b} is selected from H and protecting group (e.g. Fmoc); and
R^{3b} is selected from H, C₁-C₆ alkyl and protecting group (e.g. Pbf);
R^{8b} is selected from H and protecting group (e.g. C₁-C₆ alkyl);
wherein the group comprising the amino acid moiety is appended to a carbon atom on the aryl ring at any chemically suitable position.

In preferred examples, R^{1b} may be selected from H and/or protecting group and R^{8b} is H.

There is further provided a peptide comprising a residue derived from a modified amino acid according to any one of formula (I″), (Ia″) and (Ib″).

### Definitions

In the discussion above, reference is made to a number of terms, which are to be understood to have the meanings provided below, unless a context indicates to the contrary. The nomenclature used herein for defining compounds, in particular the compounds described herein, is intended to be in accordance with the rules of the International Union of Pure and Applied Chemistry (IUPAC) for chemical compounds, specifically the “IUPAC Compendium of Chemical Terminology (Gold Book)” (see A. D. Jenkins et al., Pure & Appl. Chem., 68, 2287-2311 (1996)). For the avoidance of doubt, if an IUPAC rule is contrary to a definition provided herein, the definition herein is to prevail.

The disclosure also includes various deuterated forms of the compounds disclosed herein, or of any of the Formulae disclosed herein, including Formulae (I), (Ia) and (II) (inc. corresponding subgeneric formulae defined herein) and example compounds (1) to (16), respectively, or a pharmaceutically acceptable salt and/or a corresponding tautomer form thereof (including subgeneric formulae, as defined above) of the present disclosure. Each available hydrogen atom attached to a carbon atom may be independently present as a deuterium atom. A person of ordinary skill in the art will know how to synthesize deuterated forms of the compounds of any of the Formulae disclosed herein, including Formulae (I), (Ia) and (II) (inc. corresponding subgeneric formulae defined herein) and example compounds (1) to (16), respectively, or a pharmaceutically acceptable salt and/or a corresponding tautomer form thereof (including subgeneric formulae, as defined above) of the present disclosure. For example, deuterated materials, such as alkyl groups may be prepared by conventional techniques (see for example: methyl-d₃-amine available from Aldrich Chemical Co., Milwaukee, WI, Cat. No. 489, 689-2).

The disclosure also includes isotopically-labelled compounds which are identical to those recited in any of the Formulae disclosed herein, including Formulae (I), (Ia) and (II) (inc. corresponding subgeneric formulae defined herein) and example compounds (1) to (16), respectively, or a pharmaceutically acceptable salt and/or a corresponding tautomer form thereof (including subgeneric formulae, as defined above) of the present disclosure but for the fact that one or more atoms are present as an atom having an atomic mass or mass number different from the atomic mass or mass number most commonly found in nature. Examples of isotopes that can be incorporated into compounds of the disclosure include isotopes of hydrogen, carbon, nitrogen, oxygen, fluorine, iodine and chlorine such as ³H, ¹¹C, ¹⁴C ¹⁸F, ¹²³I or ¹²⁵I. Compounds of the present disclosure and pharmaceutically acceptable salts of said compounds that contain the aforementioned isotopes and/or other isotopes of other atoms are within the scope of the present disclosure.

It should be noted that the terms “comprise”, “comprising” and/or “comprises” is/are used to denote that aspects and embodiments of this invention “comprise” a particular feature or features. It should be understood that this/these terms may also encompass aspects and/or embodiments which “consist essentially of” or “consist of” the relevant feature or features.

### DETAILED DESCRIPTION

The present application will now be further described, by way of example only, with reference to the following Figures:

FIG. 1(a) shows the structural importance of arginine residues in cell penetrating peptides (CPPs) and in particular the attributes and potential interactions of arginine residues when incorporated into CPPs.

FIG. 1(b) shows exemplar CPP scaffolds.

FIG. 1(c) shows an exemplary modified amino acid residue in accordance with the present disclosure. In particular, the arginine residues in an exemplary CPP have been replaced by an amino acid comprising a benzamidine moiety in place of a guanidine group in the side chain. CPPs comprising such modified amino acids may sometimes be referred to herein as “AmCPPs”).

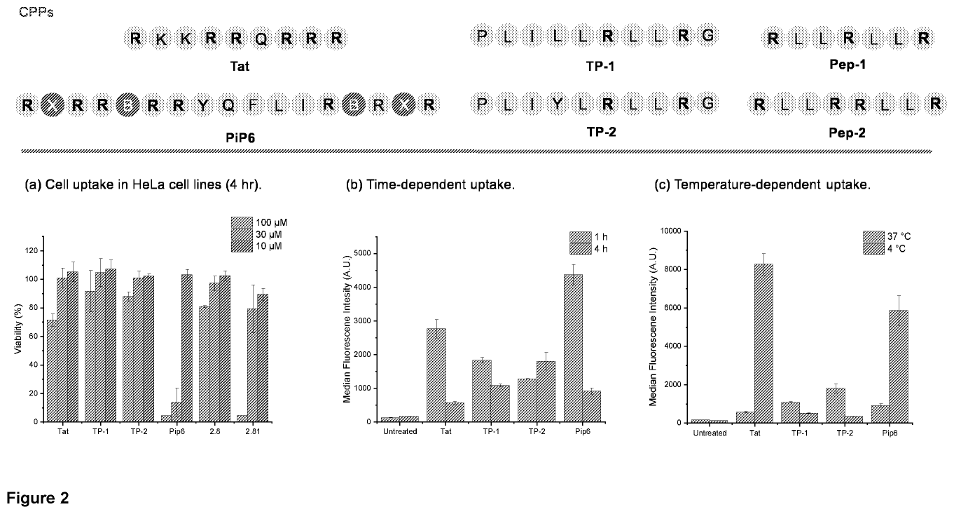

FIG. 2 shows (a) Cell viability of CPPs in HeLA cell lines. (b) Time-dependence of uptake at 1 hr and 4 hr. (c) Temperature-dependence of uptake at 37° C. and 4° C. All CPPs were fluorescently-tagged with fluorescein.

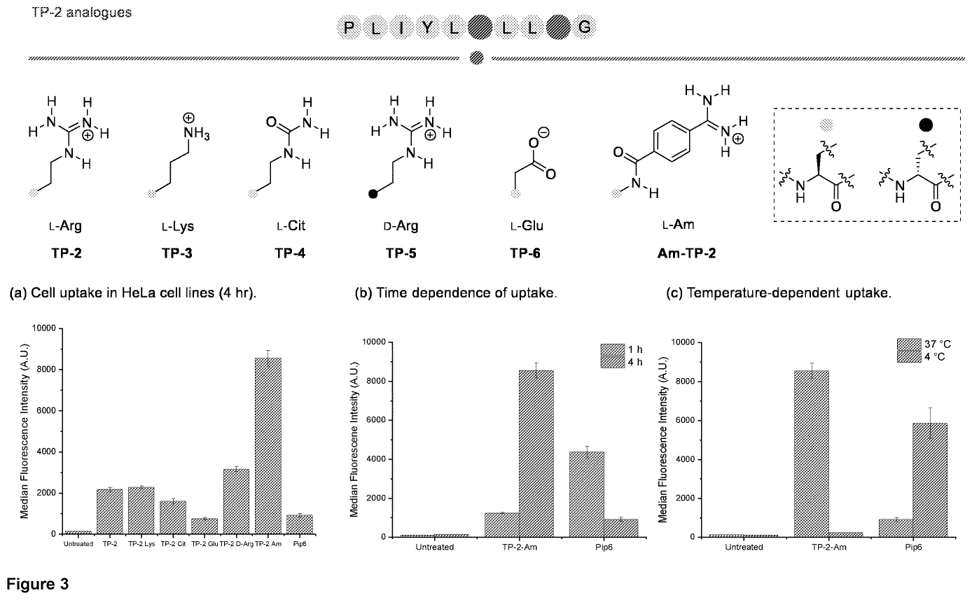

FIG. 3 shows (a) Cell uptake profile of fluorescein-tagged TP2 analogues in HeLa cell lines. (b) Time-dependence of uptake at 1 hr and 4 hr. (c) Temperature-dependence of uptake at 37° C. and 4° C.

FIG. 4 shows confocal microscopic images of the cellular distribution profile of fluorescein-tagged CPPs used in this study (a) Tat; (b) PiP6; (c) TP-1; (d) TP-2; (e) TP-2 (no Hoechst); and (f) Am-TP-2.

### EXAMPLES

### General Information

All reagents and solvents were obtained from commercial suppliers and were used without further purification. All reactions were carried out under air unless otherwise stated. Reactions were monitored by thin layer chromatography (TLC) using Merck silica plates coated with fluorescent indicator UV254. TLC plates were analysed using 254/365 nm UV light or developed using potassium permanganate solution.

### Peptide Synthesis

Peptide synthesis was completed on an automated Tribute® peptide synthesiser with an IntelliSynth UV-monitoring system and feedback control system. Rink amide resin (100-200 mesh, 0.65 mmol/g), Fmoc-Phe-OH, Fmoc-Gly-OH, Fmoc-Tyr(O^{t}Bu)-OH, Fmoc-Pro-OH, Fmoc-Leu-OH, Fmoc-Ile-OH, Fmoc-Gln(Trt)-OH, Fmoc-Arg(Pbf)-OH, Fmoc-D-Arg(Pbf)-OH, Fmoc-Cit-OH, Fmoc-Lys(Boc)-OH and Fmoc-EAhx-OH were purchased from Merck Millipore or Fluorochem and used without further purification. Fluorescein 5-isothiocyanate (FITC) was purchased from Sigma-Aldrich and used without further purification.

### Analytical HPLC

RP-HPLC was carried out using an Aeris 3.6 pm, 250×4.6 mm widepore XB C18 column using a DIONEX 3000 series HPLC equipped with a VWD3400 photodiode array detector. Samples were eluted using water (0.1% TFA) as Solvent A and acetonitrile (0.1% TFA) as Solvent B and were run at a flow rate of 1.0 mL/min.

### Analytical RP-HPLC Method A:

Absorbance detection was set to 220 nm.

TABLE S1
Gradient used for analytical RP-HPLC method A.

| Time (min) | Solvent A | Solvent B |
|---|---|---|
| 0 | 95% | 5% |
| 5 | 95% | 5% |
| 50 | 10% | 90% |
| 55 | 10% | 90% |
| 60 | 95% | 5% |

### Purification of Products

Normal-phase flash chromatography was carried out using ZEOprep 60 HYD 40-63 pm silica gel. Semi-preparative reversed-phase HPLC purification was carried out on a Kinetex 5 μm, 150×21.2 mm XB C18 column using a DIONEX 3000 series HPLC equipped with a VWD3400 variable wavelength detector. Purifications were performed using water (0.1% TFA) as Solvent A and acetonitrile (0.1% TFA) as Solvent B and were run at a flow rate of 12.0 mL/min.

### RP-HPLC Method A:

Absorbance detection was set to 220 nm.

TABLE S2
Gradient used for RP-HPLC method A.

| Time (min) | Solvent A | Solvent B |
|---|---|---|
| 0 | 95% | 5% |
| 5 | 95% | 5% |
| 45 | 10% | 90% |
| 50 | 10% | 90% |
| 53 | 95% | 5% |

### Analysis of Products

Fourier-Transform Infra-Red (FTIR) spectra were obtained on a Shimadzu IRAffinity-1 spectrometer. ¹⁹F NMR spectra were obtained on a Bruker AVANCE 400 spectrometer at 376 MHz. ¹H and ¹³C NMR spectra were obtained on either a Bruker AVANCE 400 at 400 MHz and 125 MHz, respectively, or Bruker DRX 500 at 500 MHz and 126 MHz, respectively. Chemical shifts are reported in ppm and coupling constants are reported in Hz. High-resolution mass spectra were recorded on a Bruker microTOF II mass spectrometer at the University of Edinburgh.

### Synthesis of Amidine Amino Acid Building Blocks (S4, S5a and S2b):

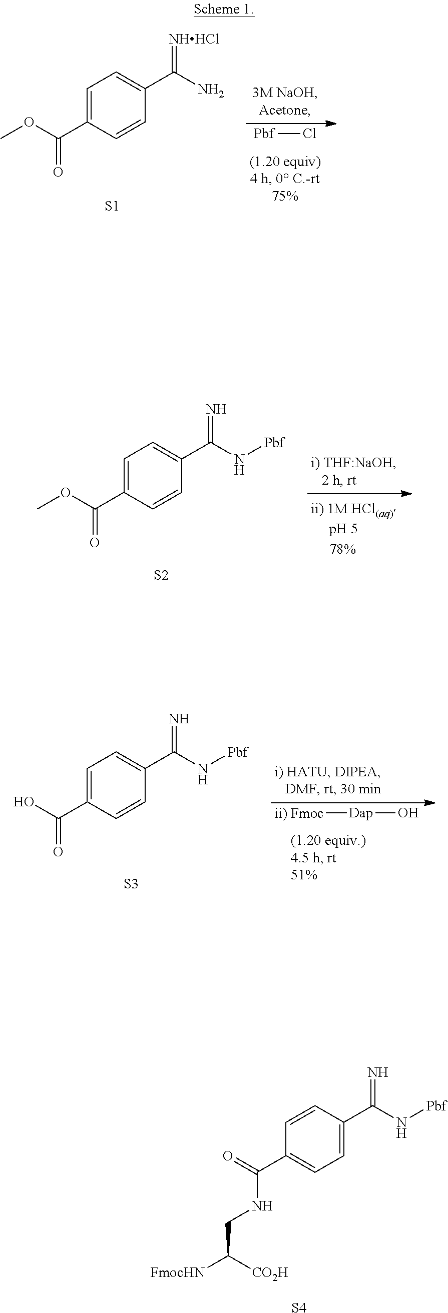

Synthetic route used to synthesise amidine amino acid S4.

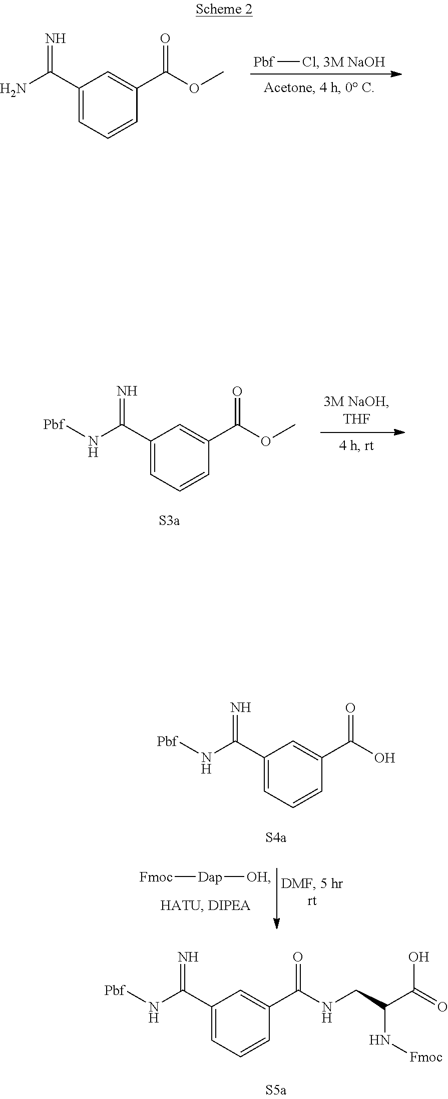

Synthetic route used to synthesise a modified amino acid S5a.

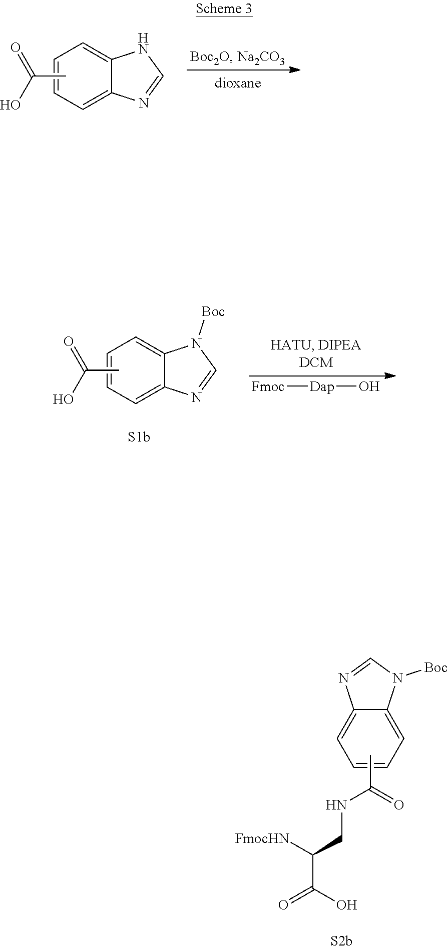

Synthetic route used to synthesise modified amino acid S2b.

### Synthetic Procedures

### Methyl 4-(N-((2,2,4,6,7-pentamethyl-2,3-dihydrobenzofuran-5-yl)sulfonyl)carbamimidoyl)benzoate (S2)

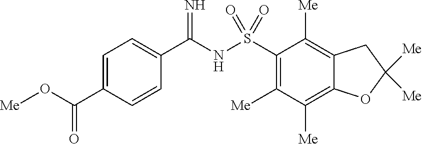 [SMILES: COC(=O)c1ccc(C(=N)NS(=O)(=O)c2c(C)c(C)c3c(c2C)CC(C)(C)O3)cc1]

Methyl 4-carbamimidoylbenzoate hydrochloride (2.00 g, 9.32 mmol) was suspended in acetone (40.0 mL) at 0° C. before the addition of 3 M NaOH solution (6.00 mL, 18.0 mmol) to form a clear solution. Separately Pbf chloride (3.23 g, 11.2 mmol) was dissolved in acetone (13.5 mL) then added dropwise to the reaction mixture. The resulting pale yellow solution was stirred at 0° C. for 2 h until a white suspension formed, the reaction was then stirred for a further 2 h at room temperature (rt). The reaction was subsequently acidified to pH 6 with 3 M HCl solution and the suspension filtered. The solid was washed with water (15 mL) then acetone (15 mL). The filtrate was then extracted with ethyl acetate (EtOAc) (3×50 mL) and the combined organic layers were then washed with brine (100 mL) before being dried over MgSO₄. The organic solution was then concentrated in vacuo and the resulting residue purified by SiO₂ chromatography in gradient of 0% to 40% EtOAc in petroleum ether (40-60) to afford a colourless solid (3.03 g, 75%). mp 400° C. degraded.

¹H NMR (500 MHz, CDCl₃): δ/ppm: 8.21 (br s, 1H, NH), 8.05 (d, 2H, J=8.5 Hz, H₃CO—C(O)—C_{q}—CH×2), 7.82 (d, 2H, J=8.5 Hz, H₃CO—C(O)—C_{q}—CH—CH×2), 6.22 (br s, 1H, NH), 3.92 (s, 3H, C_{q}—C(O)—OCH₃), 2.96 (s, 2H, O—C_{q}(CH₃)₂—CH₂), 2.62 (s, 3H, S(O)₂—C_{q}-C_{q}—(CH₃)—C_{q}—(CH₃)), 2.55 (s, 3H, S(O)₂—C_{q}-C_{q}—(CH₃)—C_{q}), 2.10 (s, 3H, S(O)₂—C_{q}-C_{q}(CH₃)—C_{q}—(CH₃)), 1.46 (s, 6H, C_{q}—CH₃×2).

¹³C{¹H}NMR (125 MHz, CDCl₃): δ/ppm: 166.0 (C_{q}), 160.2 (C_{q}), 159.6 (C_{q}), 139.3 (C_{q}), 137.9 (C_{q}), 133.5 (C_{q}), 133.2 (C_{q}), 130.9 (C_{q}), 129.9 (2×C—H), 127.3 (2×C—H), 124.9 (C_{q}), 117.8 (C_{q}), 86.7 (C_{q}), 52.4 (CH₃), 43.1 (CH₂), 28.6 (2×CH₃), 19.2 (CH₃), 17.9 (CH₃), 12.4 (CH₃).

IR (neat): vₘₐₓ/cm⁻¹: 3450 (N—H stretch), 3334 (N—H stretch), 2973 (C—H stretch), 2929 (C—H stretch), 1703 (C═O stretch), 1628 (C═N stretch), 1578 (C═C stretch), 1533 (C═C stretch).

HRMS (ESI): Calc. for C₂₂H₂₇N₂O₅S⁺. Theoretical: 431.1635 Observed: 431.1637.

### 4-(N-((2,2,4,6,7-pentamethyl-2,3-dihydrobenzofuran-5-yl)sulfonyl)carbamimidoyl)benzoic acid (S3)

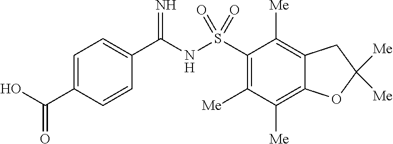 [SMILES: Cc1c(C)c(S(=O)(=O)NC(=N)c2ccc(C(=O)O)cc2)c(C)c2c1OC(C)(C)C2]

Amidine S2 (2.50 g, 5.81 mmol) was dissolved in tetrahydrofuran (THF) (29.1 mL) and then 1 M NaOH solution (29.1 mL, 29.1 mmol) was added dropwise and the resulting solution was stirred at rt for 2 h. The THF was removed in vacuo and the reaction was diluted with water (25 mL). The aqueous solution was then acidified to pH 5 with 1 M HCl solution to form an off-white gum. The suspension was filtered and the solid washed with water (25 mL). The colourless solid was then dried under high vacuum before being purified by SiO₂ chromatography in gradient of 0% to 50% EtOAc in petroleum ether (40-60) with 1% acetic acid (AcOH) modifier to afford a colourless solid (1.90 g, 78%). mp 400° C. degraded.

¹H NMR (500 MHz, DMSO-d6): δ/ppm: 13.23 (br s, 1H, CO₂h), 8.98 (br s, 1H, NH), 8.06 (br s, 1H, NH), 8.00 (d, 2H, J=8.7 Hz, HO₂C—C_{q}—CH×2), 7.91 (d, 2H, J=8.7 Hz, HO₂C—C_{q}—CH—CH×2), 3.00 (s, 2H, O—C_{q}(CH₃)₂—CH₂), 2.52 (s, 3H, S(O)₂—C_{q}-C_{q}—(CH₃)—C_{q}—(CH₃)), 2.46 (s, 3H, S(O)₂—C_{q}-C_{q}—(CH₃)—C_{q}), 2.04 (s, 3H, S(O)₂—C_{q}-C_{q}(CH₃)—C_{q}—(CH₃)), 1.43 (s, 6H, C_{q}—CH₃×2). ¹³C{¹H}NMR (125 MHz, DMSO-d6): δ/ppm: 167.1 (C_{q}), 160.9 (C_{q}), 158.8 (C_{q}), 138.5 (C_{q}), 138.0 (C_{q}), 134.2 (C_{q}), 132.7 (C_{q}), 132.5 (C_{q}), 129.8 (2×C—H), 128.4 (2×C—H), 125.3 (C_{q}), 117.2 (C_{q}), 87.2 (C_{q}), 42.7 (CH₂), 28.7 (2×CH₃), 19.3 (CH₃), 18.1 (CH₃), 12.7 (CH₃).

IR (neat): vₘₐₓ/cm⁻¹: 3358 (N—H stretch), 3223 (N—H stretch), 3091 (CO₂H stretch), 2972 (C—H stretch), 2937 (C—H stretch), 1710 (C═O stretch), 1670 (C═N stretch), 1589 (C═C stretch), 1533 (C═C stretch).

HRMS (ESI): Calc. for C₂₁H₂₅N₂O₅S⁺. Theoretical: 417.1479 Observed: 417.1474.

### (S)-2-((((9H-fluoren-9-yl)methoxy)carbonyl)amino)-3-(4-(N-((2,2,4,6,7-pentamethyl-2,3-dihydrobenzofuran-5-yl)sulfonyl)carbamimidoyl)benzamido)propanoic acid (S4)

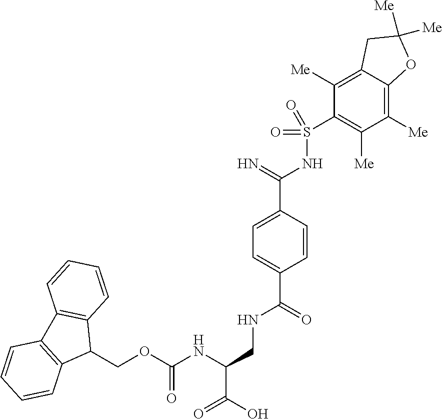 [SMILES: Cc1c(C)c(S(=O)(=O)NC(=N)c2ccc(C(=O)NC[C@H](NC(=O)OCC3c4ccccc4-c4ccccc43)C(=O)O)cc2)c(C)c2c1OC(C)(C)C2]

Amidine S3 (1.10 g, 2.64 mmol) and HATU (1.00 g, 2.64 mmol) were dissolved in DMF (26.4 mL) and then N,N-diisopropylethylamine (DIPEA) (920 μL, 5.28 mmol) was added dropwise and the resulting yellow solution was stirred at room temperature (rt) for 30 min. The reaction became a dark orange colour to which Fmoc-Dap-OH (1.15 g, 3.17 mmol) was added portion wise and the reaction mixture was stirred at rt for 4.5 h. EtOAc (80 mL) and water (80 mL) were added and the organic layer separated. The aqueous layer was then extracted with EtOAc (2×80 mL). The combined organic layers were then washed with brine (150 mL), dried over MgSO₄ then filtered, before being concentrated in vacuo. The resulting residue was purified by SiO₂ chromatography in a gradient of 0% to 4% MeOH in DCM with 1% AcOH modifier to afford a colourless solid (905 mg, 51%). mp 113-117° C.

[α]_{D}=−16.86 (c=0.011 g mL⁻¹, MeOH)

¹H NMR (500 MHz, CD₃OD): δ/ppm: 7.76 (d, 2H, J=8.6 Hz, HN═C_{q}-C_{q}—CH—CH×2), 7.72 (d, 2H, J=8.6 Hz, HN═C_{q}—C_{q}—CH×2), 7.62 (dd, 2H, J=7.5, 3.3 Hz, CO₂—CH₂—CH—C_{q}—CH—CH—CH—CH×2), 7.50 (d, 2H, J=7.5 Hz, CO₂—CH₂—CH—C_{q}—CH×2), 7.22 (2H, m, CO₂—CH₂—CH—C_{q}—CH—CH—CH×2), 7.12 (2H, m, CO₂—CH₂—CH—C_{q}—CH—CH×2), 4.41 (1H, m, HO₂C—CH), 4.19 (2H, m, CO₂—CH₂), 4.06 (1H, t, J=7.1 Hz, CO₂—CH₂—CH) 3.74 (1H, m, HO₂C—CH—CH_{A}HB), 3.63 (1H, m, HO₂C—CH—CH_{A}HB), 2.91 (s, 2H, O—C_{q}(CH₃)₂—CH₂), 2.49 (s, 3H, S(O)₂—C_{q}-C_{q}—(CH₃)— C_{q}—(CH₃)), 2.44 (s, 3H, S(O)₂—C_{q}-C_{q}—(CH₃)—C_{q}), 1.99 (s, 3H, S(O)₂—C_{q}-C_{q}(CH₃)—C_{q}—CH₃), 1.35 (s, 6H, C_{q}—CH₃×2).

¹³C{¹H}NMR (125 MHz, CD₃OD): δ/ppm: 172.1 (C_{q}), 168.3 (C_{q}), 161.6 (C_{q}), 159.2 (C_{q}), 157.2 (C_{q}), 143.8 (C_{q}), 143.7 (C_{q}), 141.1 (2×C_{q}), 138.7 (C_{q}), 137.4 (C_{q}), 136.6 (C_{q}), 132.9 (C_{q}), 131.3 (C_{q}), 127.6 (2×C—H), 127.4 (2×C—H), 127.2 (2×C—H), 126.7 (2×C—H), 125.0 (C_{q}), 124.8 (2×C—H), 119.5 (2×C—H), 117.3 (C_{q}), 86.6 (C_{q}), 66.7 (CH₂), 53.8 (CH), 46.8 (CH), 42.4 (CH₂), 40.9 (CH₂), 27.3 (2×CH₃), 18.0 (CH₃), 16.9 (CH₃), 11.0 (CH₃).

IR (neat): vₘₐₓ/cm⁻¹: 3394 (N—H stretch), 3311 (N—H stretch), 3115 (CO₂H stretch), 2972 (C—H stretch), 2927 (C—H stretch), 1716 (C═O stretch), 1705 (C═O stretch), 1627 (C═N stretch), 1575 (C═C stretch), 1527 (C═C stretch).

HRMS (ESI): Calc. for C₃₉H₄₁N₄O₈S⁺. Theoretical: 725.2640 Observed: 725.2624.

### Methyl 3-(N-((2,2,4,6,7-pentamethyl-2,3-dihydrobenzofuran-5-yl)sulfonyl)carbamimidoyl) benzoate (S3a)

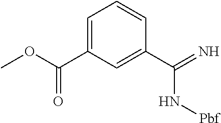

Methyl 3-carbamimidoylbenzoate hydrochloride (200 mg, 932 umol) was suspended in acetone (4.0 mL) at 0° C. before the addition of 3 M NaOH solution (621 uL, 18.0 mmol) to form a clear solution. Separately Pbf chloride (323 mg, 1.12 mmol) was dissolved in acetone (1.35 mL) then added dropwise to the reaction mixture. The resulting solution was stirred at 0° C. for 2 h and was then stirred for a further 2 h at rt. The reaction was subsequently acidified to pH 6 with HCl (3M aq.), then extracted with EtOAc (5×10 mL) and the combined organic layers were dried (MgSO₄). The crude mixture was then purified by column chromatography (3:2, Hex:EtOAc) to give the title compound as a colourless solid (260 mg, 65%). mp 400° C. degraded.

¹H NMR (500 MHz, d₆-DMSO): δ/ppm: 9.05 (s, 1H, NH), 8.38 (s, 1H, NH), 8.12 (d, 1H, J=7.8 Hz, ArH), 8.10-8.02 (m, 2H, 2×ArH), 7.63 (t, 1H, J=7.8 Hz, ArH), 3.88 (s, 3H, OCH₃), 2.99 (s, 2H, CH₂), 2.50 (s, 3H, CH₃— under DMSO), 2.45 (s, 3H, CH₃), 2.03 (s, 3H, CH₃), 1.42 (s, 6H, 2×CH₃).

¹³C{¹H}NMR (125 MHz, d₆-DMSO): δ/ppm: 166.0 (C_{q}), 160.9 (C_{q}), 158.8 (C_{q}), 138.47 (C_{q}), 134.6 (C_{q}), 132.9 (C_{q}), 132.7 (CH), 132.7 (C_{q}), 132.5 (C_{q}), 130.4 (CH), 129.7 (CH), 128.9 (CH), 125.38 (C_{q}), 117.2 (C_{q}), 87.27 (C_{q}), 52.9 (CH₃), 42.7 (CH₂), 28.7 (2×CH₃), 19.3 (CH₃), 18.0 (CH₃), 12.7 (CH₃).

m/z: ESI+: 453.5 ([M+Na]⁺, 18%), 431.3 ([M+H]⁺, 100).

### 3-(N-((2,2,4,6,7-pentamethyl-2,3-dihydrobenzofuran-5-yl)sulfonyl)carbamimidoyl)benzoic acid (S4a)

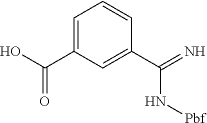

S3a (200 mg, 465 umol) was dissolved in THF (2.3 mL) and then 1 M LiOH solution (2.3 mL, 9.20 mmol) was added dropwise and the resulting solution was stirred at rt for 2 h. The THF was removed in vacuo and the reaction was diluted with water (5 mL). The aqueous solution was then acidified to pH 5 with HCl (1M aq.), extracted with EtOAc (5×10 mL) and DCM (5×5 mL), the organic layers dried (MgSO₄) and concentrated in vacuo. The crude mixture was then purified by column chromatography (4:1, Chloroform:EtOH) to give the title compound as a colourless solid (176 mg, 91%).

¹H NMR (500 MHz, d₆-DMSO): δ/ppm: 13.22 (br s, 1H, COOH), 9.03 (s, 1H, NH), 8.37 (s, 1H, NH), 8.10 (d, 1H, J=7.8 Hz, ArH), 8.07-7.98 (m, 2H, 2×ArH), 7.60 (t, 1H, J=7.8 Hz, ArH), 2.99 (s, 2H, CH₂), 2.50 (s, 3H, CH₃— under DMSO), 2.46 (s, 3H, CH₃), 2.03 (s, 3H, CH₃), 1.42 (s, 6H, 2×CH₃).

¹³C{¹H}NMR (125 MHz, d₆-DMSO): δ/ppm: 166.6 (C_{q}), 160.5 (C_{q}), 158.3 (C_{q}), 138.0 (C_{q}), 134.0 (C_{q}), 132.6 (C_{q}), 132.2 (CH), 132.1 (C_{q}), 131.8 (CH), 131.1 (C_{q}), 129.0 (CH), 128.6 (CH), 124.9 (C_{q}), 116.7 (C_{q}), 86.8 (C_{q}), 42.3 (CH₂), 28.3 (2×CH₃), 18.8 (CH₃), 17.5 (CH₃), 12.2 (CH₃).

m/z: ESI+: 439.2 ([M+Na]⁺, 30%), 417.2 ([M+H]⁺, 100).

### (S)-2-((((9H-fluoren-9-yl)methoxy)carbonyl)amino)-3-(3-(N-((2,2,4,6,7-pentamethyl-2,3-dihydrobenzofuran-5-yl)sulfonyl)carbamimidoyl)benzamido)propanoic acid (S5a)

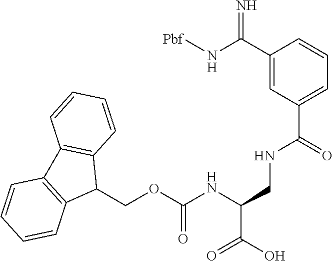

S4a (150 mg, 360 μmol) and HATU (137 mg, 360 μmol) were placed under an inert atmosphere and dry DCM (1.50 mL) added. DIPEA (125 μL, 720 μmol) was then added dropwise whereupon the mixture was then stirred for 30 mins and the initial suspension dissolved. Solid Fmoc-Dap-OH (141 mg, 432 μmol) was then added portionwise to maintain solubility and stirring. The reaction was then stirred for ~18 h, the volatiles removed, H₂O (5 mL) added and the pH adjusted to 5 with HCl (1M aq.). The aq. mixture was then extracted with EtOAc (5×20 mL) and DCM (5×10 mL), the combined organic layers dried (MgSO₄), filtered and concentrated in vacuo. The crude mixture was then purified by column chromatography (7:3, Chloroform:EtOH), taken up in H₂O:ACN and freeze dried to give the title compound as a colourless solid (142 mg, 54%).

¹H NMR (500 MHz, d₆-DMSO): δ/ppm: 8.92 (s, 1H, NH), 8.67 (s, 1H, CONH), 8.25 (s, 1H, ArH), 8.03 (s, 1H, NH), 7.94 (dd, 2H, J=13.2, 7.9 Hz, 2×ArH), 7.87 (d, 2H, J=7.5 Hz, 2×Fmoc-ArH), 7.67 (d, 2H, J=7.5 Hz, 2×Fmoc-ArH), 7.56 (t, 1H, J=7.8 Hz, ArH), 7.42-7.36 (m, 2H, 2×Fmoc-ArH), 7.32-7.24 (m, 2H, 2×Fmoc-ArH), 4.30-4.25 (m, 2H, Fmoc-CH₂), 4.22-4.18 (m, 1H, Fmoc-CH), 4.15 (br s, 1H, αCH), 3.62-3.57 (m, 2H, βCH₂), 2.98 (s, 2H, Pbf-CH₂), 2.50 (s, 3H, CH₃— under DMSO), 2.45 (s, 3H, CH₃), 2.02 (s, 3H, CH₃), 1.41 (s, 6H, 2×CH₃).

¹³C{¹H}NMR (125 MHz, d₆-DMSO): δ/ppm: 165.7 (C_{q}), 160.8 (C_{q}), 158.3 (2×C_{q}), 143.8 (2×C_{q}), 140.7 (2×C_{q}), 138.0 (C_{q}), 134.7 (C_{q}), 133.9 (C_{q}), 132.2 (C_{q}), 132.1 (C_{q}), 130.4 (CH), 130.3 (CH), 128.6 (CH), 127.6 (2×CH), 127.1 (2×CH), 126.8 (CH), 125.2 (2×CH), 124.9 (C_{q}), 120.0 (2×CH), 116.7 (C_{q}), 86.8 (C_{q}), 65.6 (CH₂), 49.4 (CH), 46.6 (CH), 42.2 (CH₂), 41.5 (CH₂), 28.3 (2×CH₃), 18.8 (CH₃), 17.6 (CH₃), 12.2 (CH₃).

m/z: ESI+: 747.0 ([M+Na]⁺, 14%), 449.3 (20), 324.3 (70), 282.2 (100).

### 1-(tert-butoxycarbonyl)-1H-benzo[d]imidazole-(6/5)-carboxylic acid (Sib)

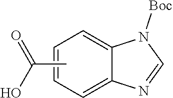

1H-benzimidazole-6-carboxylic acid (100 mg, 614 umol) was dissolved in 1,4-dioxane (1 mL) and taken to 0° C. Na₂CO₃ (1.63 mL, 10% aq.) was then added with Boc₂O (162 mg, 740 umol) added portionwise. The reaction was then allowed to warm to rt and stirred o/n, the dioxane removed under reduced pressure, H₂O (5 mL) added and the pH adjusted to 5. The aq. mixture was then extracted with EtOAc (5×10 mL), the organic layer dried (MgSO₄), filtered and concentrated in vacuo. The crude mixture was then purified by column chromatography (9:1, Chloroform:EtOH) to give the title compound as a colourless solid (162 mg, 45%).

mp 400° C. degraded.

¹H NMR (500 MHz, d₆-DMSO): δ/ppm: 13.01 (br s, 1H, COOH), 8.76 (s, 1H, NCHN), 8.27 (s, 1H, ArH), 8.03 (s, 2H, 2×ArH), 1.66 (s, 9H, 3×CH₃).

¹³C{¹H}NMR (125 MHz, d₆-DMSO): δ/ppm: 167.2 (C_{q}), 147.3 (C_{q}), 144.4 (CH), 143.5 (C_{q}), 134.1 (C_{q}), 126.9 (C_{q}), 126.2 (CH), 121.5 (CH), 114.1 (CH), 86.0 (C_{q}), 27.5 (3×CH₃).

m/z: ESI+: 263.1 ([M+H]⁺, 30%), 207.0 (40), 163.0 ([M-Boc+H]⁺, 100).

### (S)-2-((((9H-fluoren-9-yl)methoxy)carbonyl)amino)-3-(1-(tert-butoxycarbonyl)-1H-benzo[d]imidazole-(5/6)-carboxamido)propanoic acid (S2b)

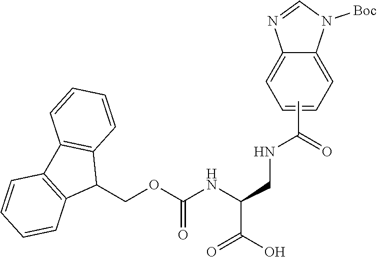

S1b (50 mg, 191 μmol) and HATU (72 mg, 191 μmol) were placed under an inert atmosphere and dry DCM (600 μL) added. DIPEA (53.0 μL, 381 μmol) was then added dropwise whereupon the mixture was then stirred for 30 mins and the initial suspension dissolved. Solid Fmoc-Dap-OH (75.0 mg, 229 μmol) was then added portionwise to maintain solubility and stirring. The reaction was then stirred for ~18 h, the volatiles removed, H₂O (5 mL) added and the pH adjusted to 5 with HCl (1M aq.). The aq. mixture was then extracted with EtOAc (5×10 mL) and DCM (5×5 mL), the combined organic layers dried (MgSO₄), filtered and concentrated in vacuo. The crude mixture was then purified by column chromatography (7:3, Chloroform:EtOH), taken up in H₂O:ACN and freeze dried to give the title compound as a colourless solid and a mixture of isomers (85.0 mg, 78%).

¹H NMR (500 MHz, d₆-DMSO): δ/ppm: 8.77-8.62 (m, 2H, NCHN & CONH), 8.56-8.44 (m, 0.5H, Iso1 ArH), 8.34-8.22 (m, 0.5H, Iso1 ArH), 8.00-7.79 (m, 4H, 2×FmocCH & 2×Iso2 ArH & Iso1/2 ArH), 7.72-7.66 (m, 2H, 2×FmocCH), 7.65-7.58 (m, 1H, OCONH), 7.45-7.33 (m, 2H, 2×FmocCH), 7.33-7.23 (m, 2H, 2×FmocCH), 4.34-4.25 (m, 3H, FmocCH₂ & αCH), 4.24-4.13 (m, 1H, FmocCH), 3.68 (br s, 2H, βCH₂), 1.71-1.60 (m, 9H, 3×CH₃).

¹³C{¹H}NMR (125 MHz, d₆-DMSO): δ/ppm: 172.2 (C_{q}), 166.6 (Iso1C_{q}), 166.4 (Iso2C_{q}), 156.0 (C_{q}), 147.4 (Iso1C_{q}), 147.3 (Iso2C_{q}), 145.7 (C_{q}), 144.8 (C_{q}), 144.1 (CH), 143.8 (Iso1C_{q}), 143.4 (Iso2C_{q}), 140.7 (C_{q}), 133.0 (C_{q}), 131.3 (C_{q}), 130.9 (Iso1C_{q}), 130.5 (Iso2C_{q}), 127.6 (2×CH), 127.1 (2×CH), 125.2 (Iso1CH), 125.2 (Iso2CH), 124.6 (CH), 123.3 (CH), 120.1 (2×CH), 119.7 (Iso1CH), 119.2 (Iso2CH), 113.8 (CH), 85.8 (Iso1C_{q}), 85.7 (Iso2C_{q}), 65.8 (CH₂), 53.9 (CH), 46.6 (CH), 40.7 (CH₂), 27.5 (Iso1 3×CH₃), 27.5 (Iso2 3×CH₃).

m/z: ESI−: 569.1 ([M−H]⁻, 100%), 424.8 (8)

### General Peptide Synthesis Protocol

On a Tribute® solid phase peptide synthesiser Rink amide resin (231 mg, 0.15 mmol, 0.65 mmol g⁻¹) was swelled in dichloromethane (DCM) (5.00 mL) for 30 min. Two 10 min agitations with 20% (v/v) solution of piperidine in dimethylformamide (DMF) (5.00 mL) was used to deprotect terminal Fmoc groups. Activation of Fmoc protected amino acids (0.45 mmol, 3.00 equiv.) was achieved using HATU (0.38 mmol, 2.50 eq.) and 0.5 M DIPEA in DMF (5.00 mL). The reaction mixture of the activated esters of Fmoc amino acids were then added sequentially to the resin and agitated for 20 min at room temperature. Upon final coupling of appropriate residue, the resin was washed with DCM (5.00 mL) and dried under N₂. The resin was then removed from the automated synthesiser and manually swelled in DCM (5.00 mL) for 30 min. After swelling the resin was washed with DMF (4×2.00 mL) and two 10 min agitations with 20% (v/v) solution of piperidine in DMF (5.00 mL) was used to deprotect the terminal Fmoc group. Separately in a 5.00 mL glass vial FITC (117 mg, 0.30 mmol) was dissolved in DMF (1.50 mL) and DIPEA (183 μL, 1.05 mmol) was added to form a bright red solution. The red FITC solution was then added to the resin, protected from light and agitated for 16 h at room temperature. After the 16 h agitation the resin was washed with DMF (4×2.00 mL) before being washed with MeOH (4×2.00 mL) and finally DCM (4×2.00 mL). The peptide was then cleaved from the resin using a mixture of TFA:phenol:water:TIPS (90:5:5:2, 3.00 mL) and agitated for 4 h. The cleavage solution was then filtered from the resin and the peptide precipitated using cold Et₂O (30 mL, −20° C.) to afford an orange precipitate. The solid was washed with Et₂O (3×10 mL) to remove excess TFA and the resulting orange solid was purified by RP-HPLC method A.

### Peptide Characterisation

FITC-XRKKRRQRRR (Tat)

(wherein X in the sequence above is a residue derived from 6-aminohexanoic acid (Ahx)).

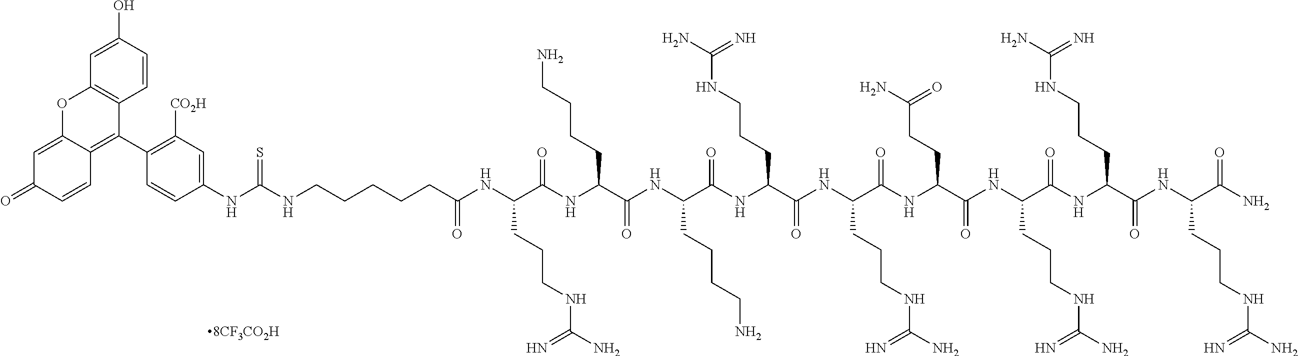 [SMILES: N=C(N)NCCC[C@H](NC(=O)[C@H](CCCNC(=N)N)NC(=O)[C@H](CCCNC(=N)N)NC(=O)[C@H](CCC(N)=O)NC(=O)[C@H](CCCNC(=N)N)NC(=O)[C@H](CCCNC(=N)N)NC(=O)[C@H](CCCCN)NC(=O)[C@H](CCCCN)NC(=O)[C@H](CCCNC(=N)N)NC(=O)CCCCCNC(=S)Nc1ccc(-c2c3ccc(=O)cc-3oc3cc(O)ccc23)c(C(=O)O)c1)C(N)=O]

Using general peptide synthesis protocol A. Appropriate HPLC fractions were lyophilised to afford a powder like orange solid (22 mg, 6%).

HRMS: C₈₀H₁₃₃N₃₃O₁₆S⁴⁺ Theoretical: 658.3235 Observed: 658.3228.

RP-HPLC (Analytical RP-HPLC method A, Kinetex 5 μm 150×21.2 mm XB C18 column) Rₜ=17.4 min, 100%.

FITC-XRLLRLLR (Pep-1)

(wherein X in the sequence above is a residue derived from 6-aminohexanoic acid (Ahx)).

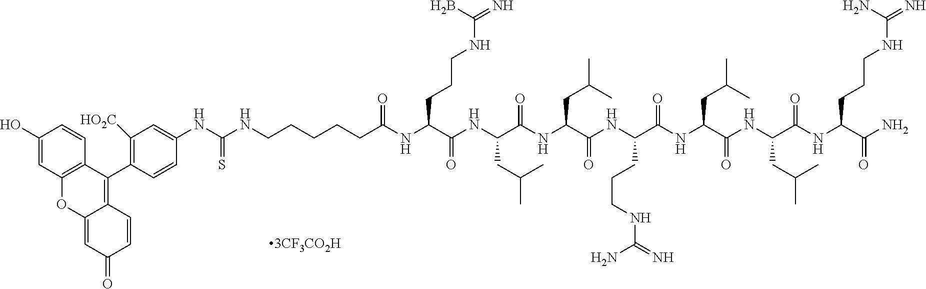

Using general peptide synthesis protocol A. Appropriate HPLC fractions were lyophilised to afford a powder like orange solid (68 mg, 25%).

HRMS: C₆₉H₁₀₇N₁₉O₁₃S²⁺ Theoretical: 720.9003 Observed: 720.9060.

RP-HPLC (Analytical RP-HPLC method A, Kinetex 5 μm 150×21.2 mm XB C18 column)

Rₜ=24.6 min, 99%.

FITC-XRLLRRLLR (Pep-2)

(wherein X in the sequence above is a residue derived from 6-aminohexanoic acid (Ahx)).

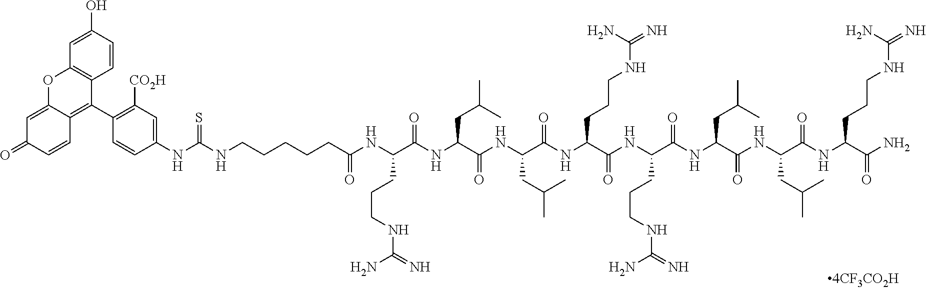 [SMILES: CC(C)C[C@H](NC(=O)[C@H](CC(C)C)NC(=O)[C@H](CCCNC(=N)N)NC(=O)[C@H](CCCNC(=N)N)NC(=O)[C@H](CC(C)C)NC(=O)[C@H](CC(C)C)NC(=O)[C@H](CCCNC(=N)N)NC(=O)CCCCCNC(=S)Nc1ccc(-c2c3ccc(=O)cc-3oc3cc(O)ccc23)c(C(=O)O)c1)C(=O)N[C@@H](CCCNC(=N)N)C(N)=O]

Using general peptide synthesis protocol A. Appropriate HPLC fractions were lyophilised to afford a powder like orange solid (70 mg, 23%).

HRMS: C₇₅H₁₁₉N₂₃O₁₄S²⁺ Theoretical: 798.9508 Observed: 798.9549.

RP-HPLC (Analytical RP-HPLC method A, Kinetex 5 μm 150×21.2 mm XB C18 column)

Rₜ=24.3 min, 98%

FITC-XRXRRBRRYQFLIRBRXR (PiP6)

(wherein X in the sequence above is a residue derived from 6-aminohexanoic acid (Ahx)).

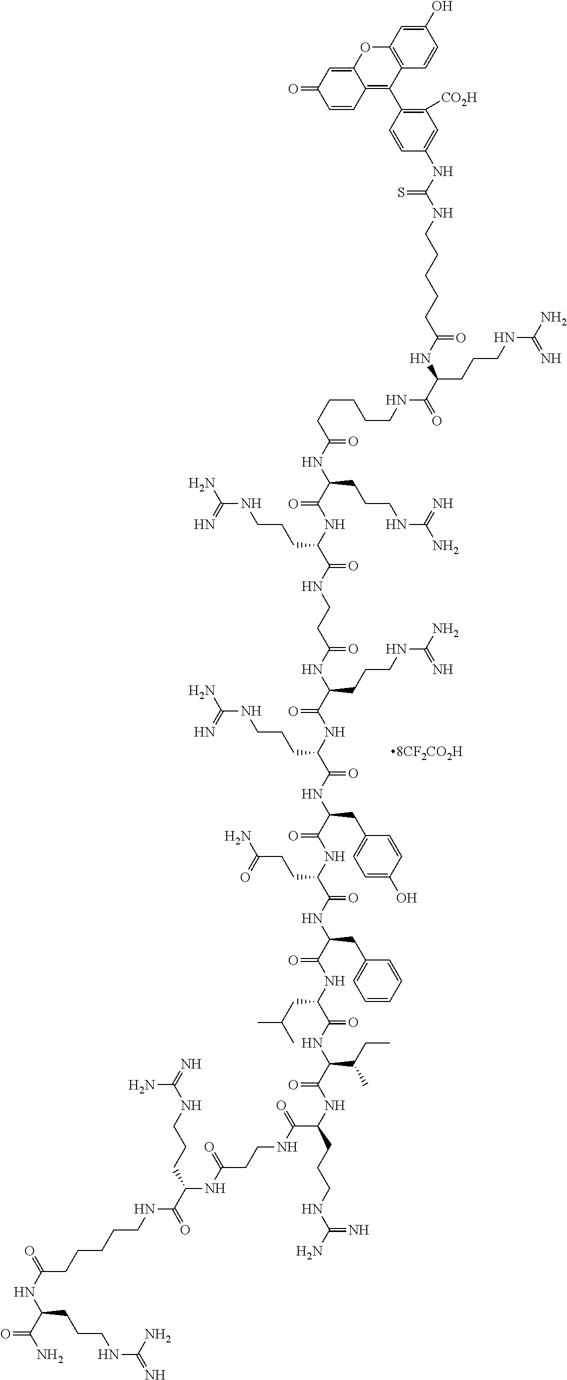 [SMILES: CC[C@H](C)[C@H](NC(=O)[C@H](CC(C)C)NC(=O)[C@H](Cc1ccccc1)NC(=O)[C@H](CCC(N)=O)NC(=O)[C@H](Cc1ccc(O)cc1)NC(=O)[C@H](CCCNC(=N)N)NC(=O)[C@H](CCCNC(=N)N)NC(=O)CCNC(=O)[C@H](CCCNC(=N)N)NC(=O)[C@H](CCCNC(=N)N)NC(=O)CCCCCNC(=O)[C@H](CCCNC(=N)N)NC(=O)CCCCCNC(=S)Nc1ccc(-c2c3ccc(=O)cc-3oc3cc(O)ccc23)c(C(=O)O)c1)C(=O)N[C@@H](CCCNC(=N)N)C(=O)NCCC(=O)N[C@@H](CCCNC(=N)N)C(=O)NCCCCCC(=O)N[C@@H](CCCNC(=N)N)C(N)=O]

Using general peptide synthesis protocol A on 0.20 mmol scale of resin. Appropriate HPLC fractions were lyophilised to afford a powder like orange solid (20 mg, 3%).

HRMS: C₁₂₈H₂₀₆N₄₅O₂₅S·CF₃CO₂H⁵⁺ Theoretical: 584.1176 Observed: 584.1211.

RP-HPLC (Analytical RP-HPLC method A, Kinetex 5 μm 150×21.2 mm XB C18 column)

Rₜ=19.7 min, 99%.

FITC-XPLILLRLLRG (TP-1)

(wherein X in the sequence above is a residue derived from 6-aminohexanoic acid (Ahx)).

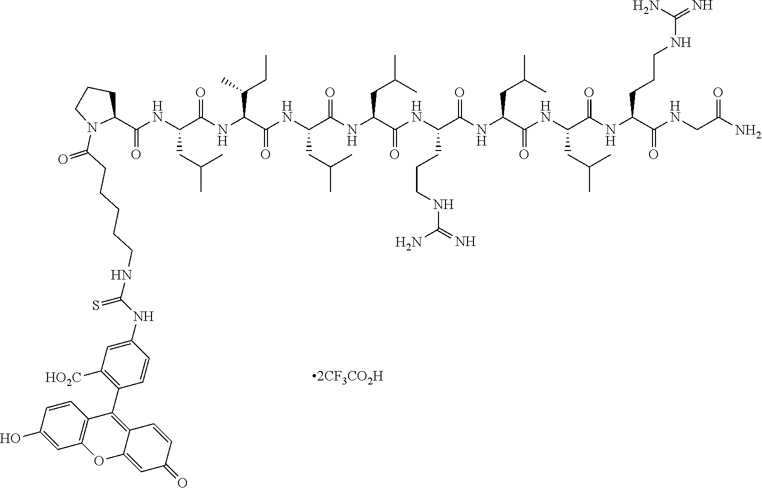 [SMILES: CC[C@@H](C)[C@H](NC(=O)[C@H](CC(C)C)NC(=O)[C@@H]1CCCN1C(=O)CCCCCNC(=S)Nc1ccc(-c2c3ccc(=O)cc-3oc3cc(O)ccc23)c(C(=O)O)c1)C(=O)N[C@@H](CC(C)C)C(=O)N[C@@H](CC(C)C)C(=O)N[C@@H](CCCNC(=N)N)C(=O)N[C@@H](CC(C)C)C(=O)N[C@@H](CC(C)C)C(=O)N[C@@H](CCCNC(=N)N)C(=O)NCC(N)=O]

Using general peptide synthesis protocol A. Appropriate HPLC fractions were lyophilised to afford a powder like orange solid (14 mg, 5%).

HRMS: C₈₂H₁₂₇N₁₉O₁₆S²⁺ Theoretical: 832.9709 Observed: 832.9710.

RP-HPLC (Analytical RP-HPLC method A, Kinetex 5 μm 150×21.2 mm XB C18 column)

Rₜ=31.3 min, 96%.

FITC-XPLIYLRLLRG (TP-2)

(wherein X in the sequence above is a residue derived from 6-aminohexanoic acid (Ahx)).

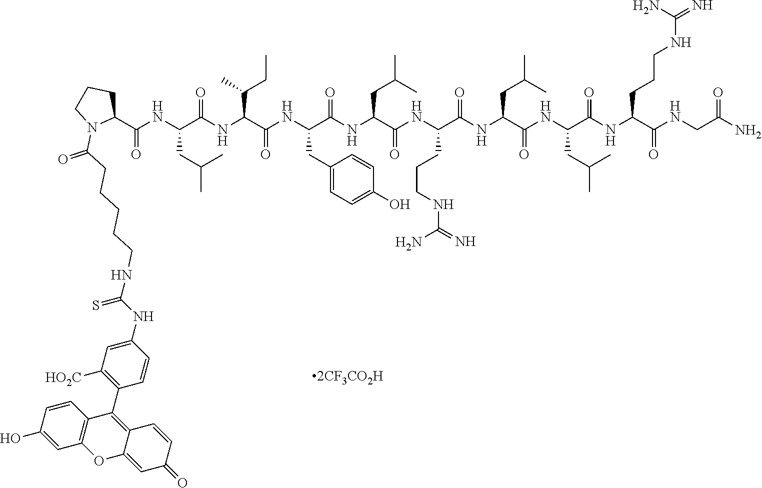 [SMILES: CC[C@@H](C)[C@H](NC(=O)[C@H](CC(C)C)NC(=O)[C@@H]1CCCN1C(=O)CCCCCNC(=S)Nc1ccc(-c2c3ccc(=O)cc-3oc3cc(O)ccc23)c(C(=O)O)c1)C(=O)N[C@@H](Cc1ccc(O)cc1)C(=O)N[C@@H](CC(C)C)C(=O)N[C@@H](CCCNC(=N)N)C(=O)N[C@@H](CC(C)C)C(=O)N[C@@H](CC(C)C)C(=O)N[C@@H](CCCNC(=N)N)C(=O)NCC(N)=O]

Using general peptide synthesis protocol A. Appropriate HPLC fractions were lyophilised to afford a powder like orange solid (19 mg, 7%).

HRMS: C₈₅H₁₂₅N₁₉O₁₇S²⁺ Theoretical: 857.9605 Observed: 857.9672.

RP-HPLC (Analytical RP-HPLC method A, Kinetex 5 μm 150×21.2 mm XB C18 column)

Rₜ=28.4 min, 97%.

FITC-XPLIYLKLLKG (TP-3)

(wherein X in the sequence above is a residue derived from 6-aminohexanoic acid (Ahx)).

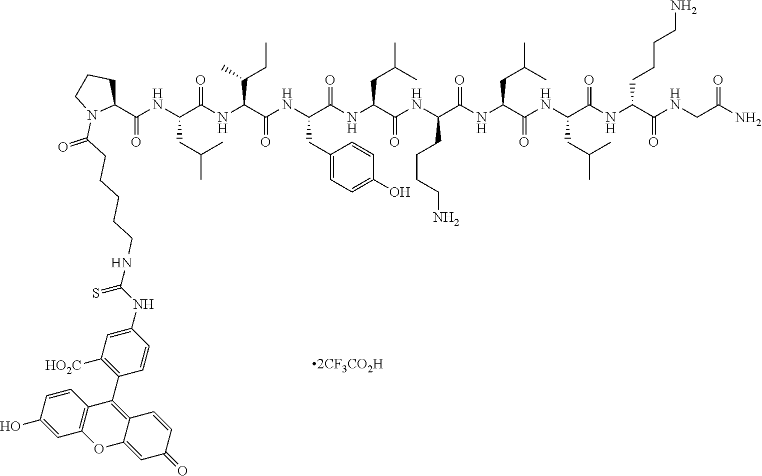 [SMILES: CC[C@@H](C)[C@H](NC(=O)[C@H](CC(C)C)NC(=O)[C@@H]1CCCN1C(=O)CCCCCNC(=S)Nc1ccc(-c2c3ccc(=O)cc-3oc3cc(O)ccc23)c(C(=O)O)c1)C(=O)N[C@@H](Cc1ccc(O)cc1)C(=O)N[C@@H](CC(C)C)C(=O)N[C@H](CCCCN)C(=O)N[C@@H](CC(C)C)C(=O)N[C@@H](CC(C)C)C(=O)N[C@H](CCCCN)C(=O)NCC(N)=O]

Using general peptide synthesis protocol A. Appropriate HPLC fractions were lyophilised to afford a powder like orange solid (17 mg, 6%).

HRMS: C₈₅H₁₂₅N₁₅O₁₇S²⁺ Theoretical: 829.9544 Observed: 829.9544.

RP-HPLC (Analytical RP-HPLC method A, Kinetex 5 μm 150×21.2 mm XB C18 column)

Rₜ=29.0 min, 97%.

FITC-XPLIYLCitLLCitG (TP-4)

(wherein X in the sequence above is a residue derived from 6-aminohexanoic acid (Ahx)).

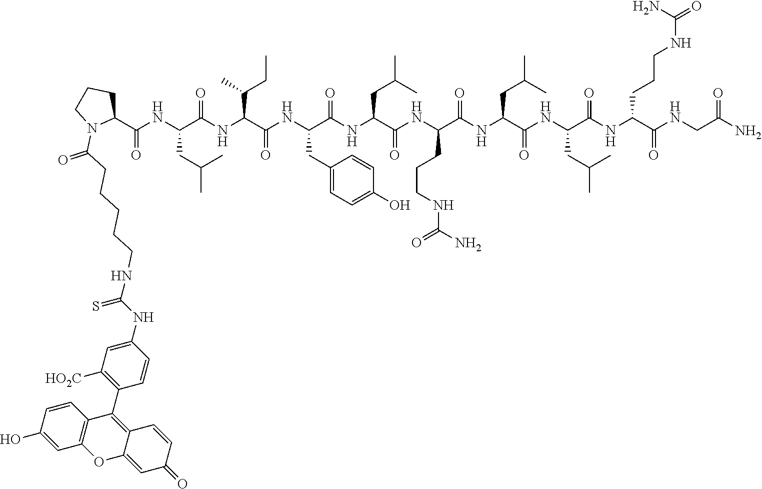 [SMILES: CC[C@@H](C)[C@H](NC(=O)[C@H](CC(C)C)NC(=O)[C@@H]1CCCN1C(=O)CCCCCNC(=S)Nc1ccc(-c2c3ccc(=O)cc-3oc3cc(O)ccc23)c(C(=O)O)c1)C(=O)N[C@@H](Cc1ccc(O)cc1)C(=O)N[C@@H](CC(C)C)C(=O)N[C@H](CCCNC(N)=O)C(=O)N[C@@H](CC(C)C)C(=O)N[C@@H](CC(C)C)C(=O)N[C@H](CCCNC(N)=O)C(=O)NCC(N)=O]

Using general peptide synthesis protocol A. Appropriate HPLC fractions were lyophilised to afford a powder like orange solid (5 mg, 2%).

HRMS: C₈₂H₁₂₃N₁₇O₁₉S2+ Theoretical: 858.9445 Observed: 858.9444.

RP-HPLC (Analytical RP-HPLC method A, Kinetex 5 μm 150×21.2 mm XB C18 column)

Rₜ=30.3 min, 100%.

FITC-XPLIYLrLLrG (TP-5)

(wherein X in the sequence above is a residue derived from 6-aminohexanoic acid (Ahx)).

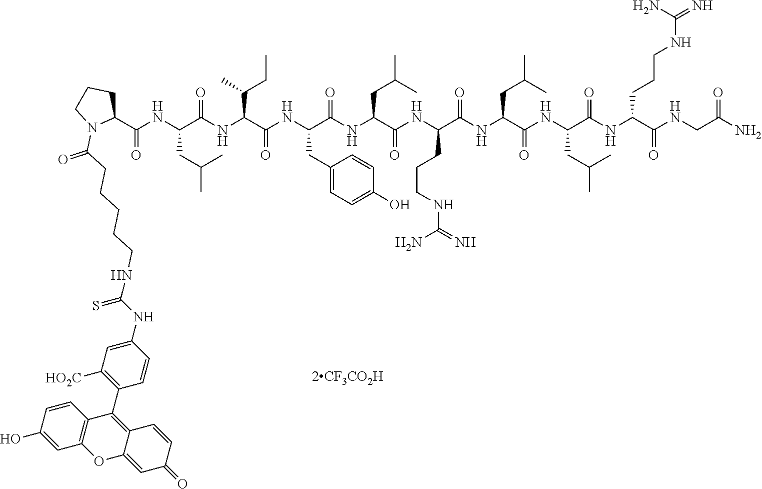 [SMILES: CC[C@@H](C)[C@H](NC(=O)[C@H](CC(C)C)NC(=O)[C@@H]1CCCN1C(=O)CCCCCNC(=S)Nc1ccc(-c2c3ccc(=O)cc-3oc3cc(O)ccc23)c(C(=O)O)c1)C(=O)N[C@@H](Cc1ccc(O)cc1)C(=O)N[C@@H](CC(C)C)C(=O)N[C@H](CCCNC(=N)N)C(=O)N[C@@H](CC(C)C)C(=O)N[C@@H](CC(C)C)C(=O)N[C@H](CCCNC(=N)N)C(=O)NCC(N)=O]

Using general peptide synthesis protocol A on a 0.10 mmol scale of resin. Appropriate HPLC fractions were lyophilised to afford a powder like orange solid (12 mg, 6%).

HRMS: C₈₅H₁₂₅N₁₉O₁₇S²⁺ Theoretical: 857.9605 Observed: 857.9600.

RP-HPLC (Analytical RP-HPLC method A, Kinetex 5 μm 150×21.2 mm XB C18 column)

Rₜ=28.6 min, 97%.

FITC-XPLIYLELLEG (TP-6)

(wherein X in the sequence above is a residue derived from 6-aminohexanoic acid (Ahx)).

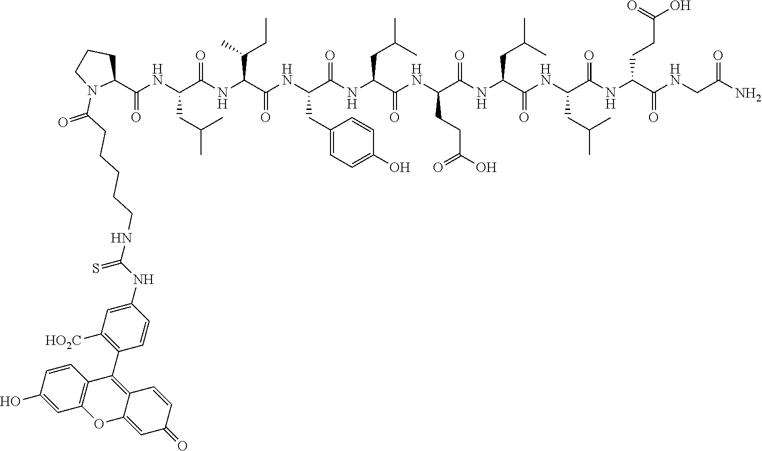 [SMILES: CC[C@@H](C)[C@H](NC(=O)[C@H](CC(C)C)NC(=O)[C@@H]1CCCN1C(=O)CCCCCNC(=S)Nc1ccc(-c2c3ccc(=O)cc-3oc3cc(O)ccc23)c(C(=O)O)c1)C(=O)N[C@@H](Cc1ccc(O)cc1)C(=O)N[C@@H](CC(C)C)C(=O)N[C@H](CCC(=O)O)C(=O)N[C@@H](CC(C)C)C(=O)N[C@@H](CC(C)C)C(=O)N[C@H](CCC(=O)O)C(=O)NCC(N)=O]

Using general peptide synthesis protocol A on a 0.75 mmol scale of resin. Appropriate HPLC fractions were lyophilised to afford a powder like orange solid (8 mg, 6%).

HRMS: C₈₃H₁₁₅N₁₃O₂₁S²⁺ Theoretical: 830.9020 Observed: 830.9004.

RP-HPLC (Analytical RP-HPLC method A, Kinetex 5 μm 150×21.2 mm XB C18 column)

Rₜ=31.1 min, 98%.

FITC-XPLIYLAmLLAmG (Am-TP-2)

(wherein X in the sequence above is a residue derived from 6-aminohexanoic acid (Ahx)).

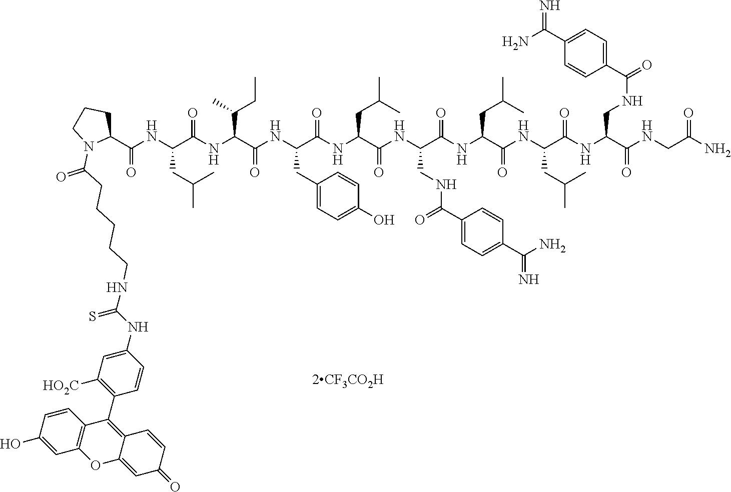 [SMILES: CC[C@@H](C)[C@H](NC(=O)[C@H](CC(C)C)NC(=O)[C@@H]1CCCN1C(=O)CCCCCNC(=S)Nc1ccc(-c2c3ccc(=O)cc-3oc3cc(O)ccc23)c(C(=O)O)c1)C(=O)N[C@@H](Cc1ccc(O)cc1)C(=O)N[C@@H](CC(C)C)C(=O)N[C@@H](CNC(=O)c1ccc(C(=N)N)cc1)C(=O)N[C@@H](CC(C)C)C(=O)N[C@@H](CC(C)C)C(=O)N[C@@H](CNC(=O)c1ccc(C(=N)N)cc1)C(=O)NCC(N)=O]

On a Tribute® solid phase peptide synthesiser Rink amide chem matrix resin (50 mg, 0.026 mmol, 0.52 mmol g⁻¹) was swelled in DCM (2.00 mL) for 30 min. Two 10 min agitations with 20% (v/v) solution of piperidine in DMF (4.00 mL) was used to deprotect terminal Fmoc groups. Activation of Fmoc protected amino acids (78.0 μmol, 3.00 equiv) was achieved using HATU (65 μmol, 2.50 eq) and 0.5 M DIPEA in DMF (2.00 mL). Activation of Fmoc Amidine S4 and Fmoc-Leu-OH position 6 was achieved with DIC (11.0 μL, 70.0 μmol, 2.70 equiv.) and HOAt (10.0 mg, 74.0 μmol, 2.85 equiv.) in DMF (2.00 mL). The reaction mixture of the activated esters of Fmoc amino acids, except amidine S4 and Fmoc-Leu-OH position 6, were then added sequentially to the resin and agitated for 20 min at room temperature. Amidine S4 and Fmoc-Leu-OH position 6 activation solutions were added to the resin and heated to 75° C. for 30 min. Upon final coupling of appropriate residue, the resin was washed with DCM (2.00 mL) and dried under N₂. The resin was then removed from the automated synthesiser and manually swelled in DCM (2.00 mL) for 30 min. After swelling the resin was washed with DMF (4×2.00 mL) and two 10 min agitations with 20% (v/v) solution of piperidine in DMF (2.00 mL) was used to deprotect the terminal Fmoc group. Separately in a 5.00 mL glass vial FITC (28 mg, 0.078 mmol) was dissolved in DMF (1.00 mL) and DIPEA (32 μL, 0.182 mmol) was added to form a bright red solution. The red FITC solution was then added to the resin, protected from light and agitated for 16 h at room temperature. After the 16 h agitation the resin was washed with DMF (4×2.00 mL) before being washed with MeOH (4×2.00 mL) and finally DCM (4×2.00 mL). The peptide was then cleaved from the resin using a mixture of TFA:phenol:water:TIPS (90:5:5:2, 3.00 mL) and agitated for 4 h at 60° C. The cleavage solution was then filtered from the resin and the peptide precipitated using cold Et₂O (30 mL, −20° C.) to afford an orange precipitate. The solid was washed with Et₂O (3×10 mL) to remove excess TFA and the resulting orange solid was purified by RP-HPLC method A. Appropriate HPLC fractions were lyophilised to afford a powder like orange solid (2 mg, 4%).

HRMS: C₉₅H₁₂₅N₁₉O₁₉S²⁺. Theoretical: 933.9554 Observed: 933.9534.

RP-HPLC (Analytical RP-HPLC method A, Kinetex 5 μm 150×21.2 mm XB C18 column)

Rₜ=28.2 min, 98%.

FITC-XPLILLrLLrG (TP-1-D-Arg)

(wherein X in the sequence above is a residue derived from 6-aminohexanoic acid (Ahx)).

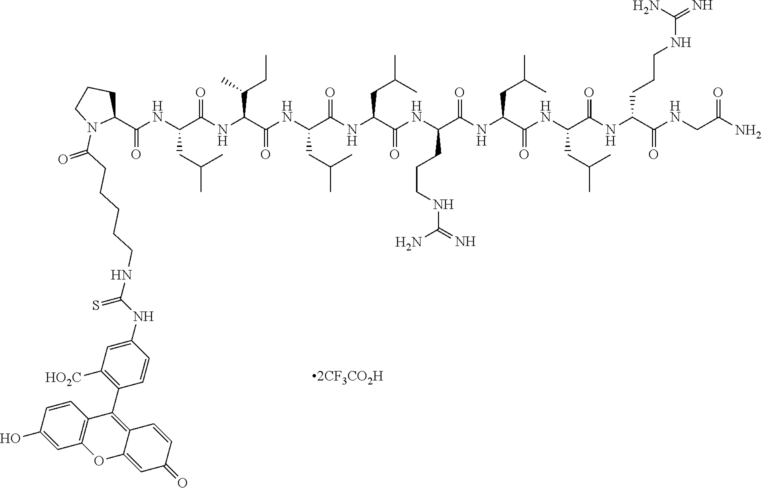 [SMILES: CC[C@@H](C)[C@H](NC(=O)[C@H](CC(C)C)NC(=O)[C@@H]1CCCN1C(=O)CCCCCNC(=S)Nc1ccc(-c2c3ccc(=O)cc-3oc3cc(O)ccc23)c(C(=O)O)c1)C(=O)N[C@@H](CC(C)C)C(=O)N[C@@H](CC(C)C)C(=O)N[C@H](CCCNC(=N)N)C(=O)N[C@@H](CC(C)C)C(=O)N[C@@H](CC(C)C)C(=O)N[C@H](CCCNC(=N)N)C(=O)NCC(N)=O]

Using general peptide synthesis protocol A. Appropriate HPLC fractions were lyophilised to afford a powder like orange solid (10 mg, 5%).

HRMS: C₈₂H₁₂₇N₁₉O₁₆S²⁺ Theoretical: 832.9709 Observed: 832.9711.

RP-HPLC (Analytical RP-HPLC method A, Kinetex 5 μm 150×21.2 mm XB C18 column)

Rₜ=30.1 min, 100%.

FITC-XPLILLKLLKG (TP-1-Lys)

(wherein X in the sequence above is a residue derived from 6-aminohexanoic acid (Ahx)).

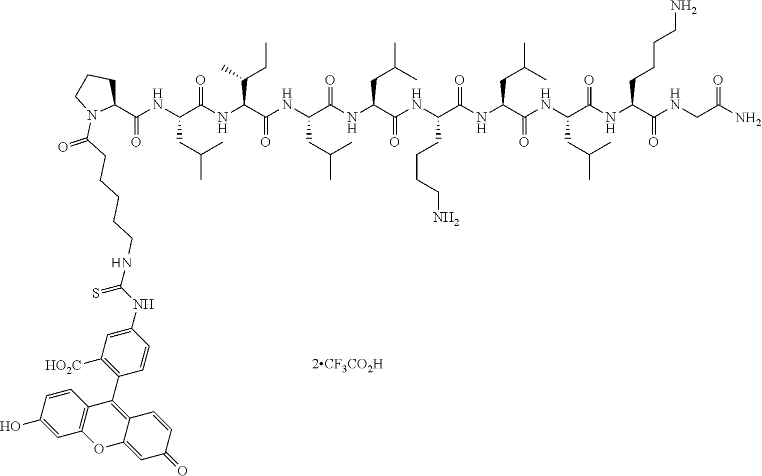 [SMILES: CC[C@@H](C)[C@H](NC(=O)[C@H](CC(C)C)NC(=O)[C@@H]1CCCN1C(=O)CCCCCNC(=S)Nc1ccc(-c2c3ccc(=O)cc-3oc3cc(O)ccc23)c(C(=O)O)c1)C(=O)N[C@@H](CC(C)C)C(=O)N[C@@H](CC(C)C)C(=O)N[C@@H](CCCCN)C(=O)N[C@@H](CC(C)C)C(=O)N[C@@H](CC(C)C)C(=O)N[C@@H](CCCCN)C(=O)NCC(N)=O]

Using general peptide synthesis protocol A. Appropriate HPLC fractions were lyophilised to afford a powder like orange solid (21 mg, 8%).

HRMS: C₈₂H₁₂₇N₁₅O₁₆S²⁺ Theoretical: 804.9648 Observed: 804.9647.

RP-HPLC (Analytical RP-HPLC method A, Kinetex 5 μm 150×21.2 mm XB C18 column)

Rₜ=30.6 min, 97%.

FITC-XPLILLCitLLCitG (TP-1-Cit)

(wherein X in the sequence above is a residue derived from 6-aminohexanoic acid (Ahx)).

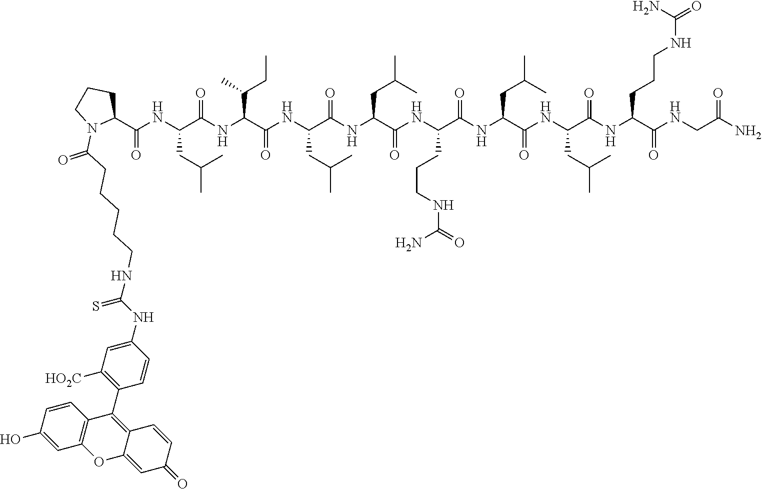 [SMILES: CC[C@@H](C)[C@H](NC(=O)[C@H](CC(C)C)NC(=O)[C@@H]1CCCN1C(=O)CCCCCNC(=S)Nc1ccc(-c2c3ccc(=O)cc-3oc3cc(O)ccc23)c(C(=O)O)c1)C(=O)N[C@@H](CC(C)C)C(=O)N[C@@H](CC(C)C)C(=O)N[C@@H](CCCNC(N)=O)C(=O)N[C@@H](CC(C)C)C(=O)N[C@@H](CC(C)C)C(=O)N[C@@H](CCCNC(N)=O)C(=O)NCC(N)=O]

Using general peptide synthesis protocol A. Appropriate HPLC fractions were lyophilised to afford a powder like orange solid (7 mg, 3%).

HRMS: C₈₂H₁₂₅N₁₇O₁₈S²⁺ Theoretical: 833.9549 Observed: 833.9548.

RP-HPLC (Analytical RP-HPLC method A, Kinetex 5 μm 150×21.2 mm XB C18 column)

Rₜ=33.7 min, 98%.

FITC-XPLILLELLEG (TP-1-Glu)

(wherein X in the sequence above is a residue derived from 6-aminohexanoic acid (Ahx)).

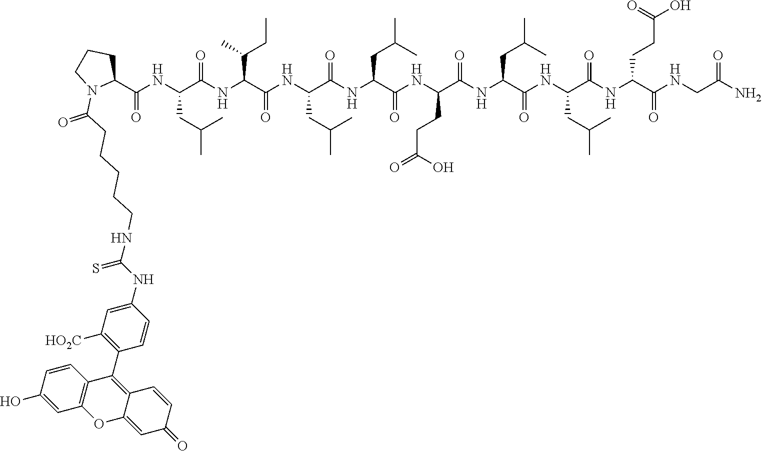 [SMILES: CC[C@@H](C)[C@H](NC(=O)[C@H](CC(C)C)NC(=O)[C@@H]1CCCN1C(=O)CCCCCNC(=S)Nc1ccc(-c2c3ccc(=O)cc-3oc3cc(O)ccc23)c(C(=O)O)c1)C(=O)N[C@@H](CC(C)C)C(=O)N[C@@H](CC(C)C)C(=O)N[C@H](CCC(=O)O)C(=O)N[C@@H](CC(C)C)C(=O)N[C@@H](CC(C)C)C(=O)N[C@H](CCC(=O)O)C(=O)NCC(N)=O]

Using general peptide synthesis protocol A on a 75.0 μmol scale of resin. Appropriate HPLC fractions were lyophilised to afford a powder like orange solid (9 mg, 7%).

HRMS: C₈₀H₁₁₇N₁₃O₂₀S²⁺ Theoretical: 805.9124 Observed: 805.9100.

RP-HPLC (Analytical RP-HPLC method A, Kinetex 5 μm 150×21.2 mm XB C18 column)

Rₜ=34.6 min, 99%.

PepS1 FITC-XPLIYLBimLLBimG (Bim-TP-2)

(wherein X is a residue derived from 6-aminohexanoic acid (Ahx); wherein Bim is a residue derived from modified amino acid S2b).

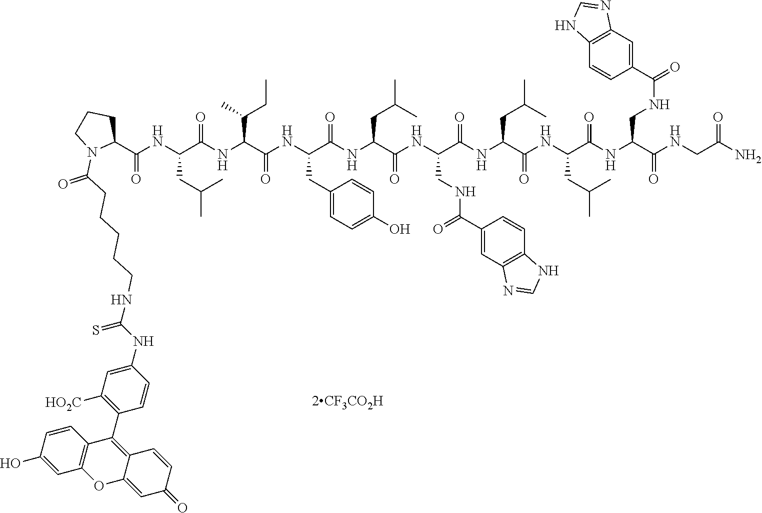 [SMILES: CC[C@@H](C)[C@H](NC(=O)[C@H](CC(C)C)NC(=O)[C@@H]1CCCN1C(=O)CCCCCNC(=S)Nc1ccc(-c2c3ccc(=O)cc-3oc3cc(O)ccc23)c(C(=O)O)c1)C(=O)N[C@@H](Cc1ccc(O)cc1)C(=O)N[C@@H](CC(C)C)C(=O)N[C@@H](CNC(=O)c1ccc2[nH]cnc2c1)C(=O)N[C@@H](CC(C)C)C(=O)N[C@@H](CC(C)C)C(=O)N[C@@H](CNC(=O)c1ccc2[nH]cnc2c1)C(=O)NCC(N)=O]

On a Biotage Alstra® solid phase peptide synthesiser Tentagel S RAM resin (100 mg, 0.025 mmol, 0.25 mmol g⁻¹) was swelled in DCM (2.00 mL) for 30 min. N-Fmoc groups were removed with two 10 min agitations with 20% (v/v) solution of piperidine in DMF. All amino acids were coupled using DIC (1 eq. cf. amino acid) and oxyma pure (1 eq. cf. amino acid) in DMF for 30 mins at 75° C. using microwave irradiation. Amino acids were used with 3 eq. excess during coupling steps. The coupling of the terminal FITC group was performed manually using FITC (7 eq.) and DIPEA (14 eq.) in DMF (1 mL) and agitated at room temperature for 16 h. After the 16 h agitation the resin was washed with DMF (4×2.00 mL) before being washed with MeOH (4×2.00 mL) and finally DCM (4×2.00 mL). The peptide was then cleaved from the resin using a mixture of TFA:phenol:water:TIPS (90:5:5:2, 3.00 mL) and agitated for 4 h at 60° C. The cleavage solution was then filtered from the resin and the peptide precipitated using cold Et₂O (30 mL, −20° C.) to afford an orange precipitate. The solid was washed with Et₂O (3×10 mL) to remove excess TFA and the resulting orange solid was purified by RP-HPLC method A. Appropriate HPLC fractions were lyophilised to afford a powder like orange solid (15 mg, 15%).

RP-HPLC (Analytical RP-HPLC method A, Aeris 2.6 pm 250×2.1 mm Peptide-XB C₁₈ column) Rₜ=17.4 min, 98%.

m/z: ESI+: 1863.0 ([M+H]⁺, 16%), 931.9 ([M+2H]²+, 100)

PepS2 FITC-XPLIYLmAmLLmAmG (mAm-TP-2)

(wherein X is a residue derived from 6-aminohexanoic acid (Ahx); wherein mAm is a residue derived from modified amino acid 5a).

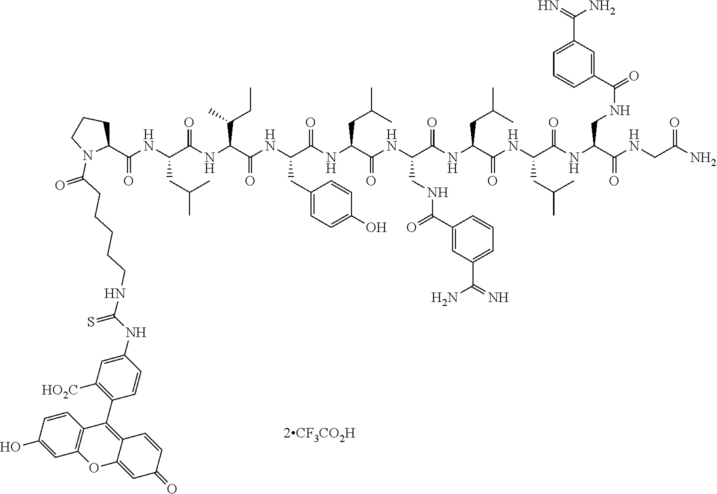 [SMILES: CC[C@@H](C)[C@H](NC(=O)[C@H](CC(C)C)NC(=O)[C@@H]1CCCN1C(=O)CCCCCNC(=S)Nc1ccc(-c2c3ccc(=O)cc-3oc3cc(O)ccc23)c(C(=O)O)c1)C(=O)N[C@@H](Cc1ccc(O)cc1)C(=O)N[C@@H](CC(C)C)C(=O)N[C@@H](CNC(=O)c1cccc(C(=N)N)c1)C(=O)N[C@@H](CC(C)C)C(=O)N[C@@H](CC(C)C)C(=O)N[C@@H](CNC(=O)c1cccc(C(=N)N)c1)C(=O)NCC(N)=O]

On a Biotage Alstra® solid phase peptide synthesiser Tentagel S RAM resin (100 mg, 0.025 mmol, 0.25 mmol g⁻¹) was swelled in DCM (2.00 mL) for 30 min. N-Fmoc groups were removed with two 10 min agitations with 20% (v/v) solution of piperidine in DMF. All amino acids were coupled using DIC (1 eq.) and oxyma pure (1 eq.) in DMF for 30 mins at 75° C. using microwave irradiation. The coupling of the terminal FITC group was performed manually using FITC (7 eq.) and DIPEA (14 eq.) in DMF (1 mL) and agitated at room temperature for 16 h. After the 16 h agitation the resin was washed with DMF (4×2.00 mL) before being washed with MeOH (4×2.00 mL) and finally DCM (4×2.00 mL). The peptide was then cleaved from the resin using a mixture of TFA:phenol:water:TIPS (90:5:5:2, 3.00 mL) and agitated for 4 h at 60° C. The cleavage solution was then filtered from the resin and the peptide precipitated using cold Et₂O (30 mL, −20° C.) to afford an orange precipitate. The solid was washed with Et₂O (3×10 mL) to remove excess TFA and the resulting orange solid was purified by RP-HPLC method A. Appropriate HPLC fractions were lyophilised to afford a powder like orange solid (7 mg, 15%).

RP-HPLC (Analytical RP-HPLC method A, Aeris 2.6 pm 250×2.1 mm Peptide-XB C₁₈ column) Rₜ=17.7 min, 95%.

m/z: ESI+: 1866.7 ([M+H]⁺, 4%), 934.0 ([M+2H]²+, 92), 623.1 ([M+3H]³+, 100)

### Cell Culture

HeLa and U2OS cells were maintained in medium consisting of Dulbecco's modified Eagle's medium (DMEM), 10% FBS, 1% L-Glutamine and 1% penicillin/streptomycin at pH 7.4. HepG2 cells were maintained in medium consisting of Dulbecco's modified Eagle's medium (DMEM) without Glucose or phenol red but with 10% FBS, 1% L-Glutamine and 1% penicillin/streptomycin at pH 7.4. Cells were cultured in a humidified incubator at 37° C. with 5% CO₂ atmosphere. PBS used was 1×PBS consisting of 137 mM NaCl, 2.7 mM KCl, 10 mM Na₂HPO₄ and 1.8 mM KH₂PO₄ which was adjusted to pH 7.4. BSA, FBS, L-Glutamine, penicillin/streptomycin and TrypLE Xpress were purchased sterile from sigma-aldrich or Thermo Fisher scientific and used without further treatment.

### Flow Cytometry

HeLa or U2OS cells were cultured in 24 well plates (4.5×10⁵ cells per well) for 24 h. On the day of the experiment, the cells were incubated with 5 μM FITC-peptide in DMEM with 10% FBS at 37° C. or 4° C. for required time. The media was then removed and wells were washed with PBS (2×500 μL per well) and then detached from the plate with TrypLE Xpress (500 μL per well). PBS containing 2% bovine serum albumin (500 μL) was added to each well and cells transferred to 5 mL polystyrene FACS tubes. Cells were centrifuged (5 min, 5000 rpm) and washed with PBS containing 2% bovine serum albumin (2×500 μL) before being suspended in PBS containing 2% bovine serum albumin (1.00 mL per FACS tube).

Cells were finally analysed on a Thermo fisher Attune NxT flow cytometer with laser excitation/emission set at 490 nm/519 nm and a flow rate of 200 μL min⁻¹. Flow cytometry data was analysed using FlowJo software with gating set at a range of 51K-285K side scatter and 251K-815K for forward scatter variance for every sample. Median fluorescence intensity values were calculated from the gating range using FlowJo.

Median fluorescence values reported were normalised by subtracting average background fluorescence at 490 nm/519 nm of a set of untreated cells (ran with every experiment) from the value of each FITC labelled peptide.

### Cell Viability AlamarBlue Assay

HeLa, U2OS or HepG2 cells were cultured in 96 well plates (2.6×10³ cells per well) for 24 h. On the day of the experiment, the cells were incubated in a 5% CO₂ atmosphere with 100 μM, 30 μM, 10 μM, 3 μM, 1 μM, 0.3 μM, 0.1 μM, FITC-peptide in DMEM with 10% FBS (100 uL total well volume) at 37° C. for 24 h. The experiments were performed in triplicate for each concentration. AlamarBlue® was then added to each well (0.5 mM, 10 μL, 10% v/v) and incubated for a further 6 h for HeLa and HepG2 cells and 24 h for U2OS cells. Cells were then analysed using a HIdex plate reader with laser excitation/emission set at 560 nm/595 nm. Fluorescence of untreated cells were used as a 100% viable control, with 1% Triton X used as a negative control, and viability of cells were calculated as a percentage of the control fluorescence values using OriginPro 2019b software.

### Confocal Microscopy

HeLa cells were cultured in 8 well plates (Ibidi plates) (3.5×10⁴ cells per well) for 24 h. On the day of the experiment, the cells were incubated in a 5% CO₂ atmosphere with 5 μM FITC-peptide in DMEM with 10% FBS (200 uL total well volume) at 37° C. for 4 h. After 3.5 h incubation, Hoescht 33258 was added at a concentration of 5.6 μM and cells incubated at 37° C. for the final 0.5 h. Cells were removed from the incubator and media removed. Each well was then washed with serum and phenol red free media (2×200 μL) then incubated with 4% formaldehyde in PBS (200 μL) for 20 min at room temperature. After incubation the 4% formaldehyde solution was removed and PBS (200 μL) added to each well. The cells were then imaged using a Leica S8 confocal microscope. Excitation/emission was set at 352 nm/461 nm for Hoescht 33258 imaging and 495 nm/519 nm for FITC labelled peptides. Images were processed using ImageJ software. Images were ‘rolling ball’ averaged across the set for each channel to remove background fluorescence.

Additional synthetic routes to further exemplary modified amino acids are shown below.

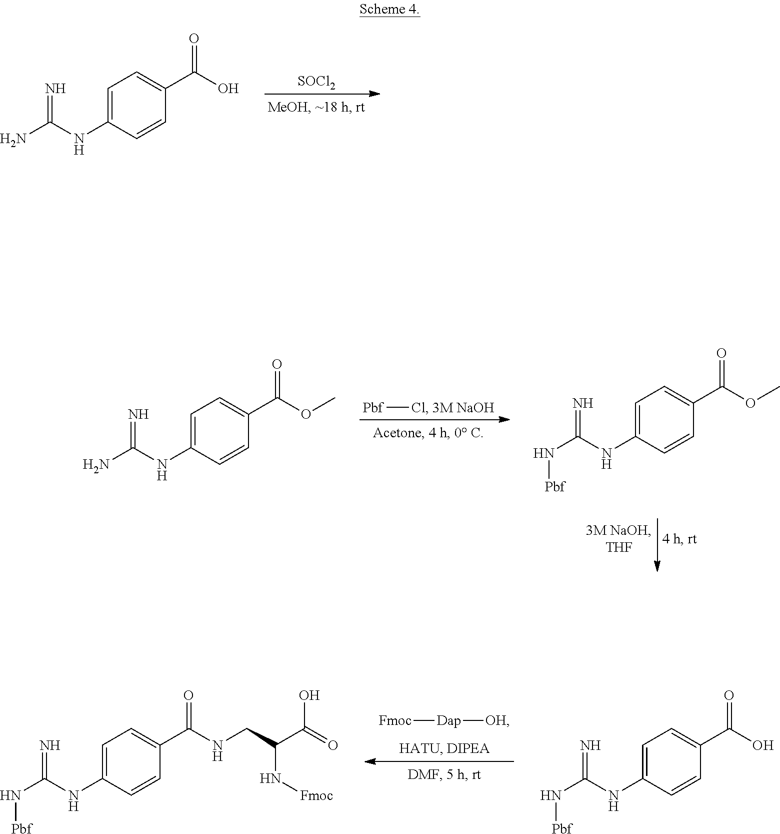

Synthetic route to synthesise a modified amino acid according to an example of the disclosure.

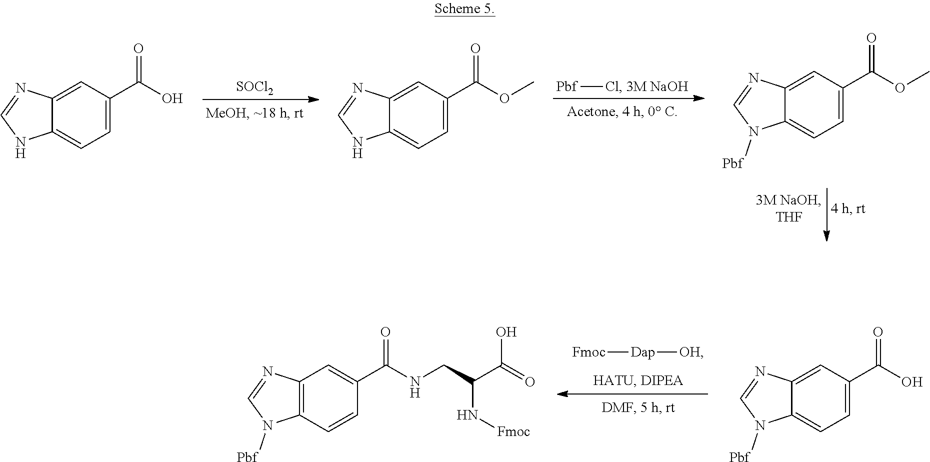

Synthetic route to synthesise a modified amino acid according to an example of the disclosure.

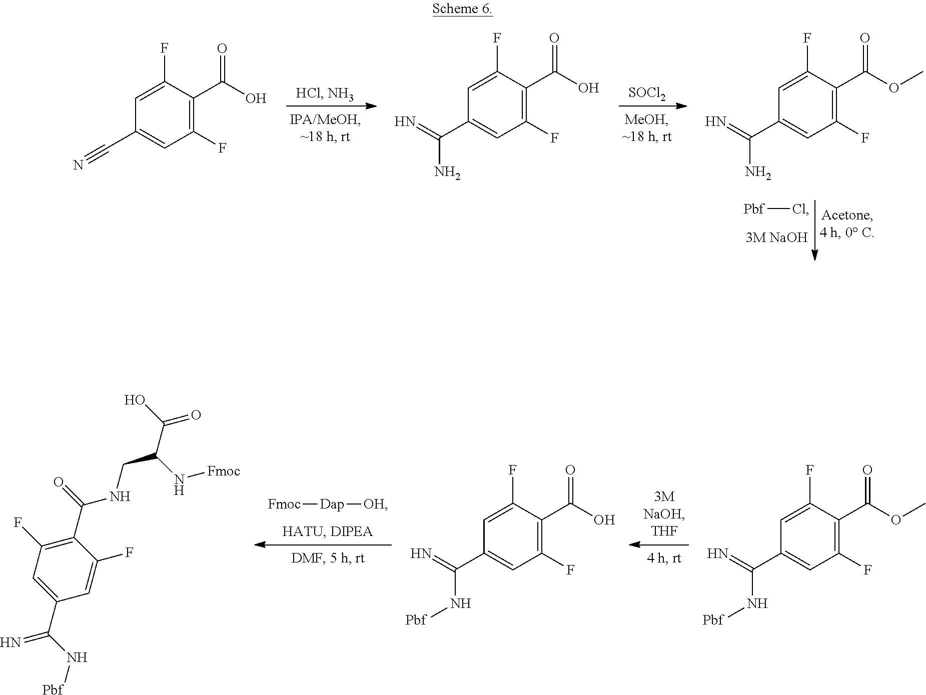

Synthetic route to synthesise a modified amino acid according to an example of the disclosure.

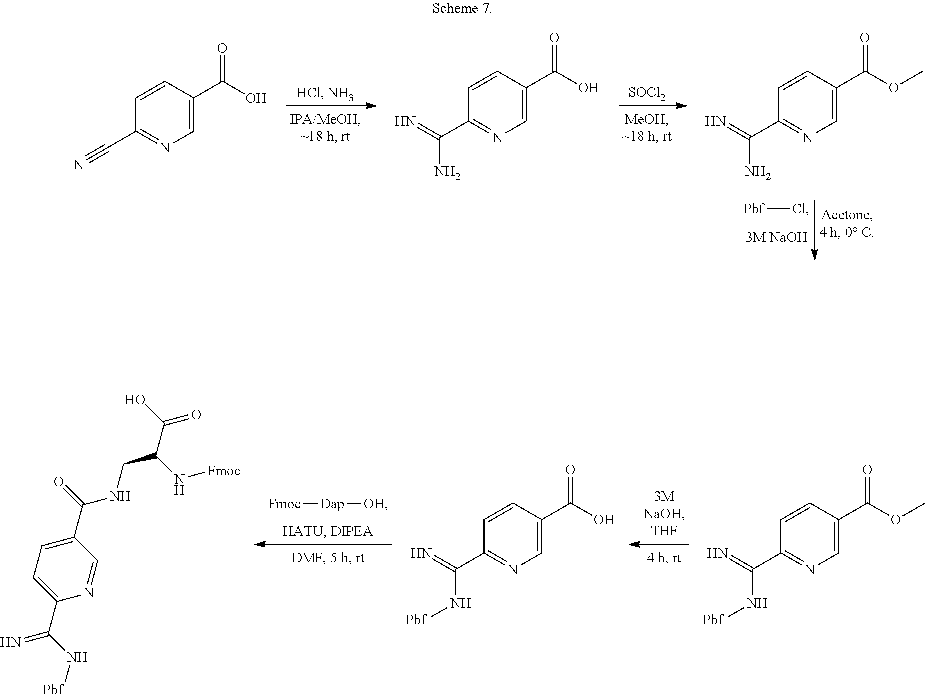

Synthetic route to synthesise a modified amino acid according to an example of the disclosure.

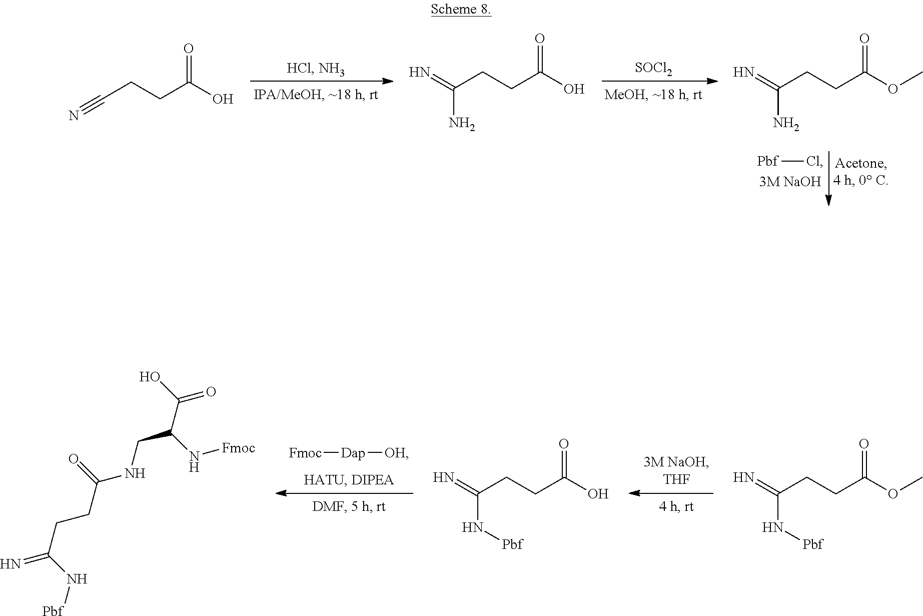

Synthetic route to synthesise a modified amino acid according to an example of the disclosure.

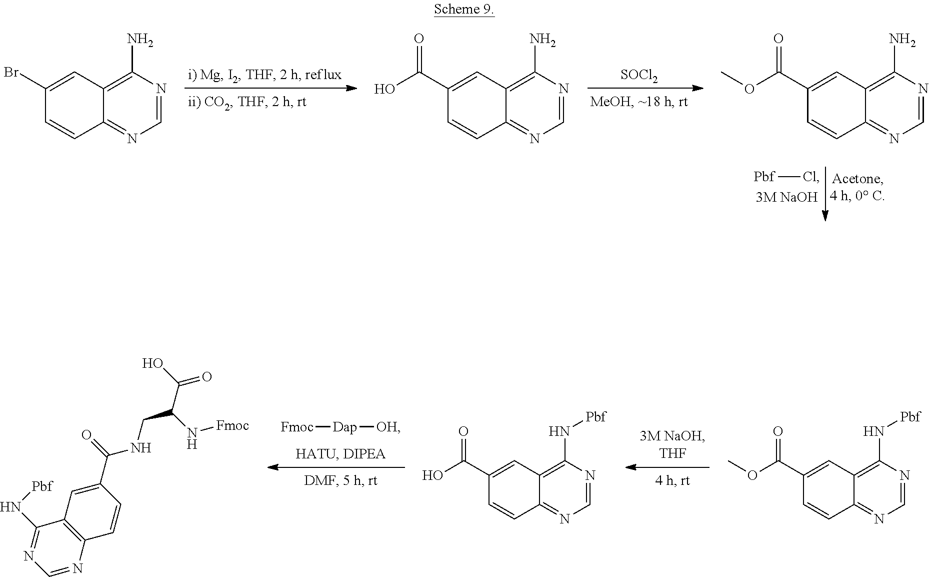

Synthetic route to synthesise a modified amino acid according to an example of the disclosure.

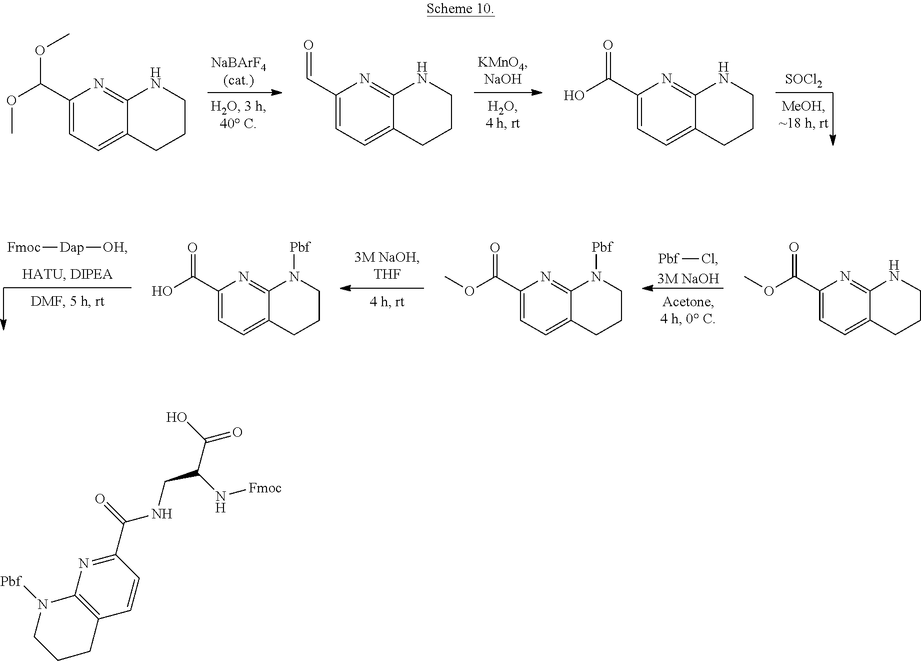

Synthetic route to synthesise a modified amino acid according to an example of the disclosure.

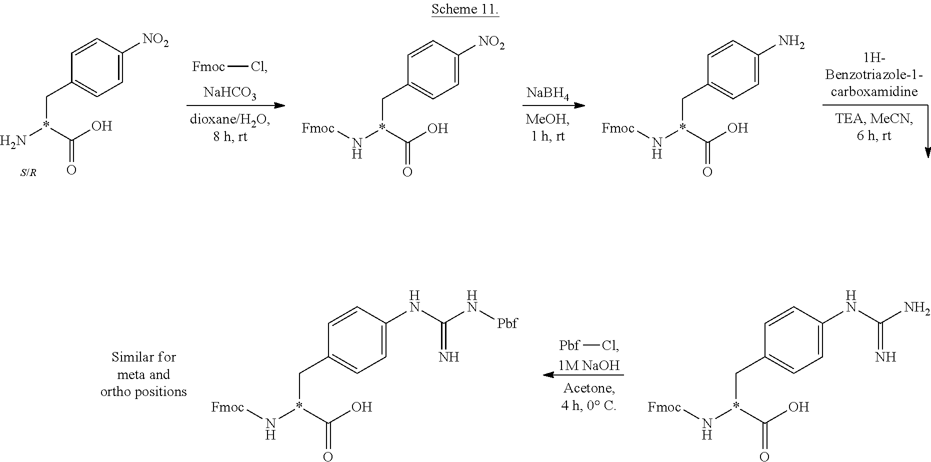

Synthetic route to synthesise a modified amino acid according to an example of the disclosure. The route is shown in the context of a compound having a para-substitution pattern on the phenyl ring. A similar synthetic route is used for analogues with ortho- and meta-substitution patterns on the phenyl ring.

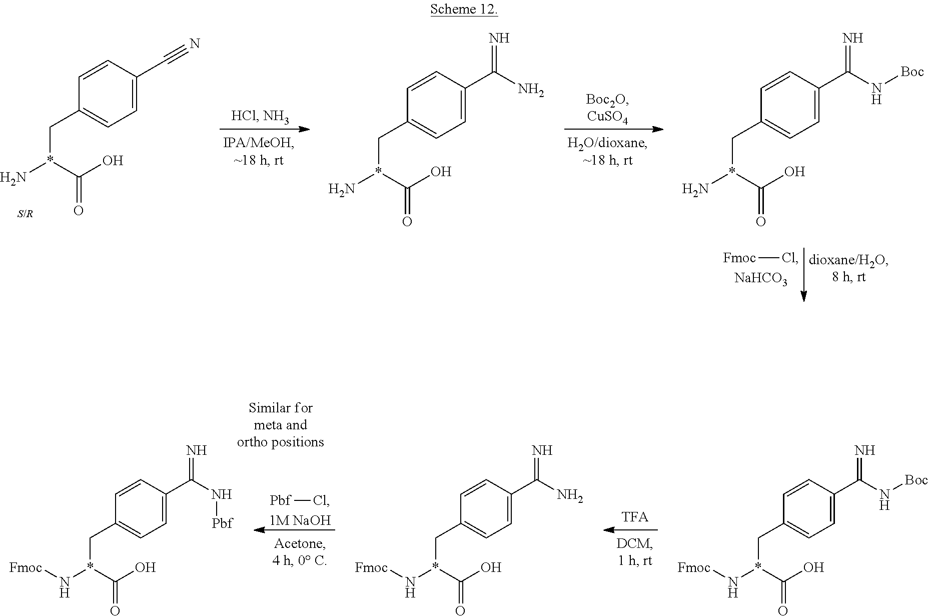

Synthetic route to synthesise a modified amino acid according to an example of the disclosure. The route is shown in the context of a compound having a para-substitution pattern on the phenyl ring. A similar synthetic route is used for analogues with ortho- and meta-substitution patterns on the phenyl ring.

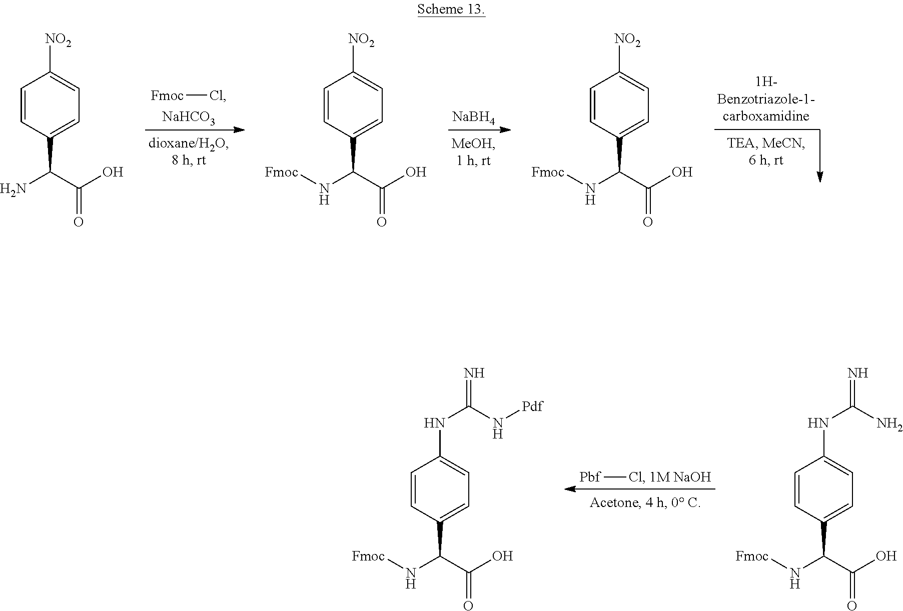

Synthetic route to synthesise a modified amino acid according to an example of the disclosure.

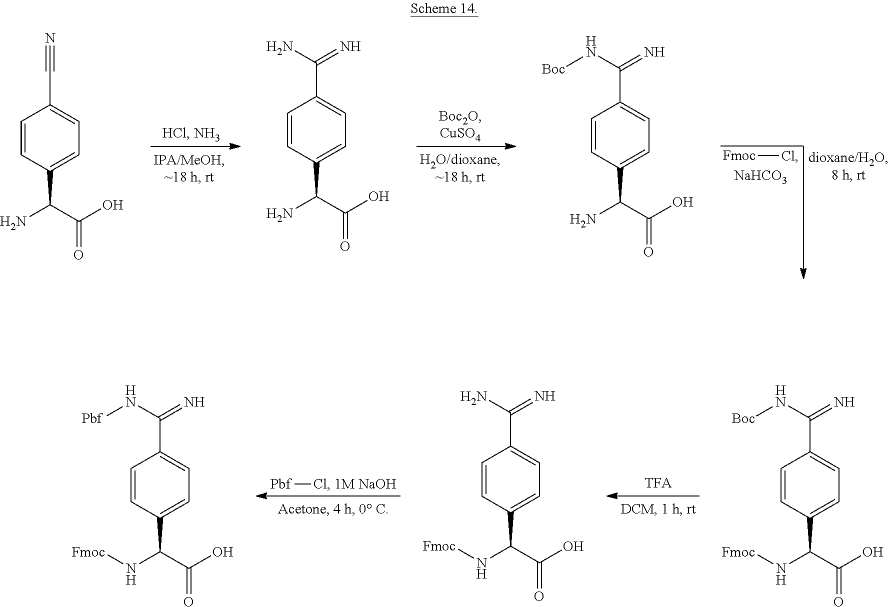

Synthetic route to synthesise a modified amino acid according to an example of the disclosure.

### Results and Discussion

In the following study, the use of amidine groups has been explored as potential arginine mimetics.

The approach was to first identify an existing cell penetrating peptide (CPP) sequence which displayed high levels of cellular uptake and distribution but minimal levels of cytotoxicity. This provided a solid foundation to explore the effect of replacing Arg residues with the Am building (such as is shown in FIG. 1c).

The three CPP exemplars chosen for the comparative studies are shown in FIG. 1b. Tat is the archetypical CPP, and has been explored extensively as a delivery tool (Green, M.; Ishino, M.; Loewenstein, P. M. Mutational analysis of HIV-1 Tat minimal domain peptides: Identification of <em>trans</em>-dominant mutants that suppress HIV-LTR-driven gene expression. Cell 1989, 58, 215-223). TP-1 and TP-2 are synthetic CPPs which were identified by high-throughput screening (Guha et al, Mechanistic Landscape of Membrane-Permeabilizing Peptides. Chem. Rev. 2019, 119, 6040-6085; and Marks et al, Spontaneous Membrane-Translocating Peptides by Orthogonal High-Throughput Screening. J. Am. Chem. Soc. 2011, 133, 8995-9004) whereas PiP6 was developed to enhance the uptake of therapeutic oligonucleotide (PMO) sequences for the treatment of Duchenne Muscular Dystrophy (Betts et al, Pip6-PMO, A New Generation of Peptide-oligonucleotide Conjugates With Improved Cardiac Exon Skipping Activity for DMD Treatment. Mol. Ther. Nucleic Acids 2012, 1).

Cell viability studies revealed the lowest cytotoxicity across all three classes of CPPs surveyed was TP-1 and TP-2, both of which were well tolerated by HeLa cells up to 100 μM (FIG. 2a). Whilst Tat displayed moderate levels of toxicity at 100 μM, cell incubated with PiP6 exhibited only 15% cell viability at 30 μM, and 5% at 100 μM. This toxicity trend was also consistent in HepG2 and U2OS cell lines. Previous work on the TP series highlighted the importance of the Arg positioning in relation to a hydrophobic core in the central Arg-Leu-Leu-Arg motif (Marks, J. R.; Placone, J.; Hristova, K.; Wimley, W. C. Spontaneous Membrane-Translocating Peptides by Orthogonal High-Throughput Screening. J. Am. Chem. Soc. 2011, 133, 8995-9004). In order to explore this further, repeating sequences of this core motif were evaluated (Pep-1, Pep-2). Cell viability studies revealed the positioning of Arg and Leu residues and the nature of the repeating units played a significant role in toxicity, with Pep-2 displaying cytotoxicity at 100 M relative to Pep-1.

Cell uptake/retention studies in HeLa cell lines revealed a time-dependent profile with Tat and PiP6 displaying maximum uptake at 1 hr at 37° C. (FIG. 2b). This suggests an energy-dependent uptake mechanism for the TP series (Madani, F.; Lindberg, S.; Langel, Ü.; Futaki, S.; Gräslund, A. Mechanisms of Cellular Uptake of Cell-Penetrating Peptides. J. Biophys. 2011, 2011, 414729) which differs from the active endocytotic mechanisms of uptake for Tat and PiP6 Guha, S.; Ghimire, J.; Wu, E.; Wimley, W. C. Mechanistic Landscape of Membrane-Permeabilizing Peptides. Chem. Rev. 2019, 119, 6040-6085; Boisguérin, P.; Deshayes, S.; Gait, M. J.; O'Donovan, L.; Godfrey, C.; Betts, C. A.; Wood, M. J. A.; Lebleu, B. Delivery of therapeutic oligonucleotides with cell penetrating peptides. Adv. Drug Deliv. Rev. 2015, 87, 52-67; Kauffman, W. B.; Guha, S.; Wimley, W. C. Synthetic molecular evolution of hybrid cell penetrating peptides. Nat. Commun. 2018, 9). This is in contrast to enhanced levels of uptake for the TP series at 4 hr; a trend which was also observed in the U2OS cell line. By virtue of enhanced cellular retention, and reduced toxicity, the TP series of peptides were taken forward for further evaluation.

The influence of both Arg residues in the TP series on toxicity, cellular uptake and distribution was then investigated. Whereas cell viability studies of TP peptides revealed very little alteration in toxicity when both Arg residues were replaced with Lys, Glu, Cit or D-Arg, analysis of the cell uptake of TP-1 and TP-2 (FIG. 3a) revealed the importance of a positive charge present at these sites. For example, TP-3 (L-Lys) showed no difference in cell uptake relative to the TP-2 archetype. A reduction in cellular uptake was then observed in TP-4 which incorporates two L-Cit residues, followed by a further reduction in uptake in TP-5 (L-Glu). Inversion of the stereochemistry to D-Arg (TP-5) enhanced uptake relative to TP peptides containing L-Arg in both positions (e.g., TP-1 and TP-2). A pertinent observation is the enhanced levels of uptake observed for the TP peptides relative to Pip6 and Tat at 4 hr. We therefore conclude that the cell uptake properties of the TP series is contingent on a positively-charged residue at both Arg positions in TP-1 and TP-2.

Uptake was further investigated using the non-natural amino acid building block (L-Am, FIG. 1c). The working hypothesis for this study was the combination of increased hydrophobicity and reduced basicity of the benzamidine building block relative to a guanidinium group would enhance uptake. Direct replacement of Arg residues with two L-Am building blocks (Am-TP-2) resulted in a remarkable 4-fold increase in uptake relative to TP-2, confirming this hypothesis (FIG. 3a).

The time- and temperature-dependency of Am-TP-2 uptake followed the same trend as that observed for TP-1 and TP-2 (FIG. 2b-c), with enhanced uptake observed at 4 hr incubation relative to 1 hr (FIG. 3b). Furthermore, cellular uptake of Am-TP-2 appears to follow a similar energy-dependent pathway as observed by a 30-fold decrease at 4° C. relative to 37° C. (FIG. 3c). Consistent with that observed for TP-1 and TP-2, the trend in enhanced uptake of Am-TP-2 was also observed in the U2OS cell line.

Finally, the cellular distribution profile of fluorescein-tagged CPPs was explored in HeLa cell lines. As confirmed in various studies previously, the distribution profile of Tat proceeds via an endocytic mechanism involving entrapment within the endosomes, which is reflected by the presence of the fluorescently-tagged Tat accumulating in puncta (FIG. 4a). The distribution profile for PiP6 is very different, and involves a high level of accumulation within the nucleus (FIG. 4b).

TP-1 (FIG. 4c) and TP-2 (FIG. 4d) are extensively distributed throughout the cytoplasm, although this distribution does extend into the nucleus but not at the same concentration as in the cytoplasm (FIG. 4e). The cytoplasmic distribution profile of the TP series is also consistent with that observed for Am-TP-2, which suggests that although the Arg residues within the TP series do influence uptake this needs to be put in context with the full sequence.

At present, the 4-fold increase in cell uptake of Am-TP-2 relative to TP-2 highlights the relative plasticity of the cell uptake profile of TP series. Although early reports of the TP series reported that the mechanism of uptake was via spontaneous translocation, the present results suggest other energy-dependent pathways might be influential, including a combination of supramolecular interactions with the phospholipid membrane as well as specific interactions with cell surface glycoproteins.

These investigations demonstrate the potential to modulate the cell penetrating properties of CPPs. In particular, these studies highlight the potential of the TP series as a CPP scaffold suitable for further evaluation as a delivery vector for large molecular cargo, potentially via the formation of bioconjugates or as part of delivery vectors such as liposomal formulations or multi-valent nanoparticles. These studies also show that the replacement of the guanidinium group of Arg with a benzamidine is not only tolerated, but actually improves the cellular uptake and distribution properties when compared to TP-2.

### REFERENCES

(1) Peraro, L.; Kritzer, J. A. Emerging Methods and Design Principles for Cell-Penetrant Peptides. Angew. Chem.-Int. Edit. 2018, 57, 11868-11881.
(2) Guidotti, G.; Brambilla, L.; Rossi, D. Cell-Penetrating Peptides: From Basic Research to Clinics. Trends Pharmacol. Sci. 2017, 38, 406-424.
(3) Dougherty, P. G.; Sahni, A.; Pei, D. H. Understanding Cell Penetration of Cyclic Peptides. Chem. Rev. 2019, 119, 10241-10287.
(4) Guha, S.; Ghimire, J.; Wu, E.; Wimley, W. C. Mechanistic Landscape of Membrane-Permeabilizing Peptides. Chem. Rev. 2019, 119, 6040-6085.
(5) Futaki, S.; Nakase, I. Cell-Surface Interactions on Arginine-Rich Cell-Penetrating Peptides Allow for Multiplex Modes of Internalization. Acc. Chem. Res. 2017, 50, 2449-2456.
(6) Brock, R. The Uptake of Arginine-Rich Cell-Penetrating Peptides: Putting the Puzzle Together. Bioconjugate Chem. 2014, 25, 863-868.
(7) Rothbard, J. B.; Jessop, T. C.; Lewis, R. S.; Murray, B. A.; Wender, P. A. Role of Membrane Potential and Hydrogen Bonding in the Mechanism of Translocation of Guanidinium-Rich Peptides into Cells. J. Am. Chem. Soc. 2004, 126, 9506-9507.
(8) Komin, A.; Russell, L. M.; Hristova, K. A.; Searson, P. C. Peptide-based strategies for enhanced cell uptake, transcellular transport, and circulation: Mechanisms and challenges. Adv. Drug Deliv. Rev. 2017, 110, 52-64.
(9) Tünnemann, G.; Ter-Avetisyan, G.; Martin, R. M.; Stöckl, M.; Herrmann, A.; Cardoso, M. C. Live-cell analysis of cell penetration ability and toxicity of oligo-arginines. J. Pept. Sci. 2008, 14, 469-476.
(10) Dinca, A.; Chien, W.-M.; Chin, M. T. Intracellular Delivery of Proteins with Cell-Penetrating Peptides for Therapeutic Uses in Human Disease. Int.l J. Mol. Sci. 2016, 17, 263.
(11) Silva, S.; Almeida, A. J.; Vale, N. Combination of Cell-Penetrating Peptides with Nanoparticles for Therapeutic Application: A Review. Biomolecules 2019, 9.
(12) Meanwell, N. A. Synopsis of Some Recent Tactical Application of Bioisosteres in Drug Design. J. Med. Chem. 2011, 54, 2529-2591.
(13) Lam, P. Y. S.; Clark, C. G.; Li, R.; Pinto, D. J. P.; Orwat, M. J.; Galemmo, R. A.; Fevig, J. M.; Teleha, C. A.; Alexander, R. S.; Smallwood, A. M.; Rossi, K. A.; Wright, M. R.; Bai, S. A.; He, K.; Luettgen, J. M.; Wong, P. C.; Knabb, R. M.; Wexler, R. R. Structure-Based Design of Novel Guanidine/Benzamidine Mimics: Potent and Orally Bioavailable Factor Xa Inhibitors as Novel Anticoagulants. J. Med. Chem. 2003, 46, 4405-4418.
(14) Flemer, S., Jr.; Madalengoitia, J. S. On-resin synthesis of novel arginine-isostere peptides bearing substituted amidine headgroups. J. Pept. Sci. 2012, 18, 30-36.
(15) Peterlin-Mašič, L.; Kikelj, D. Arginine mimetics. Tetrahedron 2001, 57, 7073-7105.
(16) Marks, J. R.; Placone, J.; Hristova, K.; Wimley, W. C. Spontaneous Membrane-Translocating Peptides by Orthogonal High-Throughput Screening. J. Am. Chem. Soc. 2011, 133, 8995-9004.
(17) Betts, C.; Saleh, A. F.; Arzumanov, A. A.; Hammond, S. M.; Godfrey, C.; Coursindel, T.; Gait, M. J.; Wood, M. J. A. Pip6-PMO, A New Generation of Peptide-oligonucleotide Conjugates With Improved Cardiac Exon Skipping Activity for DMD Treatment. Mol. Ther. Nucleic Acids 2012, 1.
(18) Madani, F.; Lindberg, S.; Langel, U.; Futaki, S.; Gräslund, A. Mechanisms of Cellular Uptake of Cell-Penetrating Peptides. J. Biophys. 2011, 2011, 414729.
(19) Kauffman, W. B.; Guha, S.; Wimley, W. C. Synthetic molecular evolution of hybrid cell penetrating peptides. Nat. Commun. 2018, 9.
(20) Fuselier, T.; Wimley, W. C. Spontaneous Membrane Translocating Peptides: The Role of Leucine-Arginine Consensus Motifs. Biophys. J. 2017, 113, 835-846.
(21) Lima de Oliveira et al, Predicting cell-penetrating peptides using machine learning algorithms and navigating in their chemical space, Nature Scientific Reports, 2021, 11, 7628.
(22) Verdine et al, Towards understanding cell penetration by stapled peptides, Med. Chem. Commun., 2015, 6, 111.
(23) Bird et al, Biophysical determinants for cellular uptake of hydrocarbon-staple peptide helices, Nature Chemical Biology, Vol. 12, October 2016, 845.

Although the present invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

1. A cell penetrating peptide comprising at least one modified amino acid residue according to formula (II): 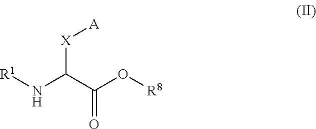
wherein R¹ is selected from H, protecting group and an amino acid residue;
R⁸ is selected from H and an amino acid residue;
X is absent, or is selected from optionally substituted C₁-C₆ alkyl, optionally substituted —C₁-C₆ alkyl-NR²(C═O)—, and optionally substituted —C₁-C₆ alkyl-(C═O)NR²—;
R² is selected from H, optionally substituted C₁-C₆ alkyl and protecting group; and
wherein A is selected from: 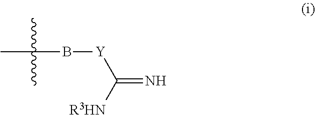
wherein B is selected from optionally substituted aryl, optionally substituted heteroaryl and optionally substituted C₁-C₆ alkyl;
Y is absent or NH;
wherein R³ is selected from H, optionally substituted C₁-C₆ alkyl and protecting group;
with the proviso that Y is absent when B is optionally substituted C₁-C₆ alkyl; or
(ii) a bicyclic fused ring system comprising an amidine-like motif according to the following formula: 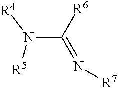
wherein R⁴ and R⁶ are each independently selected from H and a carbon atom that forms part of a backbone of the bicyclic ring system;
R⁵ is selected from H and protecting group; and
R⁷ is a carbon atom that forms part of a backbone of the bicyclic ring system;
wherein the at least one modified amino acid residue is not derived from a modified amino comprising the following structure:

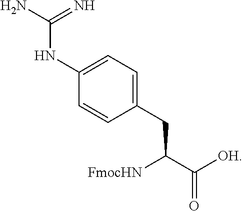

2. A cell penetrating peptide according to claim 1, wherein Y is absent.

3. A cell penetrating peptide according to claim 1, wherein at least one arginine residue has been replaced by the at least one modified amino acid according to formula (II).

4. A cell penetrating peptide according to claim 1, wherein R¹ is selected from H, amino acid residue and 9-fluorenylmethyloxycarbonyl (Fmoc).

5. A cell penetrating peptide according to claim 1, wherein X is absent or is selected from —CH₂—, —CH₂NH(C═O)— and —CH₂(C═O)NH—.

6. A cell penetrating peptide according to claim 1, wherein X is CH₂NH(C═O)—.

7. A cell penetrating peptide according to claim 1, wherein A is: 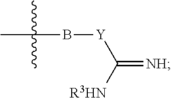
and wherein:
B is optionally substituted phenyl or optionally substituted pyridyl;
Y is absent or NH; and
R³ is optionally substituted C₁-C₆ alkyl or an acid labile protecting group.

8. A cell penetrating peptide according to claim 1, wherein the at least one modified amino acid is represented by formula (Ia′): 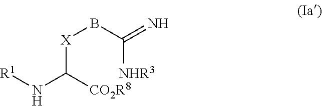
wherein
R¹ is selected from H, protecting group and an amino acid residue;
X is selected from optionally substituted C₁-C₆ alkyl, optionally substituted C₁-C₆ alkyl-NR²(C═O)— and optionally substituted —C₁-C₆ alkyl-(C═O)NR²—;
R² is selected from H, optionally substituted C₁-C₆ alkyl and protecting group;
B is selected from optionally substituted aryl and optionally substituted heteroaryl;
R³ is selected from H, optionally substituted C₁-C₆ alkyl and protecting group; and
R⁸ is selected from H and an amino acid residue.

9. A cell penetrating peptide according to claim 1, wherein A is a bicyclic fused ring system selected from the group consisting of quinazolinyl, benzimidazolyl, and tetrahydronaphthyridinyl (e.g. 1,2,3,4-tetrahydro-1,8-naphthyridinyl).

10. A cell penetrating peptide according to claim 1, wherein A is selected from: 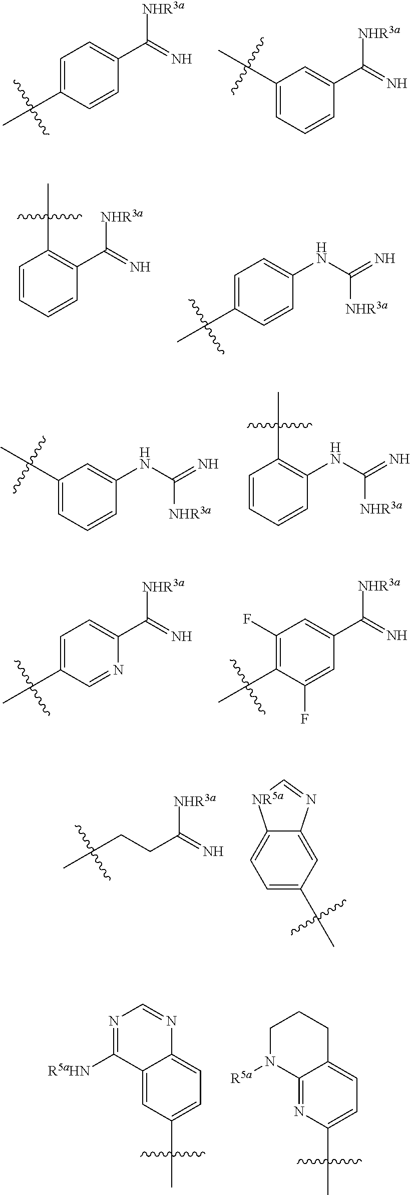
wherein R^{3a} is selected from H, C₁ to C₆ alkyl (e.g. methyl) and acid labile protecting group (e.g. 2,2,4,6,7-pentamethyl-dihydro-benzofuran-5-sulfonyl (Pbf) or Boc); and
R^{5a} is selected from H, C₁ to C₆ alkyl (e.g. methyl) and acid labile protecting group (e.g. Pbf or Boc).

11. A cell penetrating peptide according to claim 1, wherein the at least one modified amino acid comprises a structure according to formula (IIa) or formula (IIb): 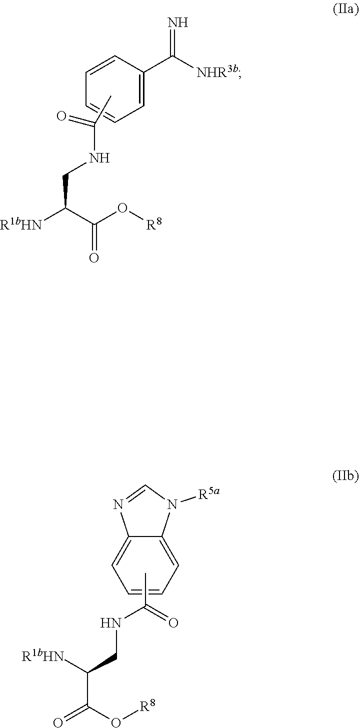
wherein R^{1b} is selected from H, protecting group (e.g. Fmoc) or amino acid residue;
R^{3b} is selected from H, C₁-C₆ alkyl and protecting group (e.g. Pbf);
R^{5a} is selected from H, C₁-C₆ alkyl and protecting group (e.g. Pbf or Boc); and
R⁸ is selected from H and an amino acid residue;
wherein the group comprising the amino acid moiety is appended to a carbon atom on the aryl ring at any chemically suitable position.

12. A cell penetrating peptide according to claim 1, wherein the cell penetrating peptide is selected from one or more of the following sequences:
(i) RKKRRQRRR—SEQ ID NO: 1
(ii) PLILLRLLRG—SEQ ID NO: 2
(iii) PLIYLRLLRG—SEQ ID NO: 3
(iv) RXRRBRRYQFLIRBRXR—SEQ ID NO: 4; wherein X is 6-aminohexanoic acid (Ahx) and B is β-alanine;
wherein one or more arginine (R) residues in each of SEQ ID NOS: 1 to 4 has been replaced with the at least one modified amino acid residue.

13. A cell penetrating peptide according to claim 1, wherein the cell penetrating peptide comprises a sequence according to SEQ ID NO: 5, SEQ ID NO: 9, SEQ ID NO: 10, or SEQ ID NO:11:
(i)
SEQ ID NO: 5
PLIYLXLLXG-
wherein each X in SEQ ID NO: 5 is selected from a modified amino acid as defined in formula (II) and/or as defined in claim 1, wherein each X may be the same or different modified amino acids;
(ii) XPLIYLAmLLAmG—SEQ ID NO: 9
wherein X in SEQ ID NO: 9 is a residue derived from 6-aminohexanoic acid (Ahx) and each Am is a modified amino acid residue according to the following structure: 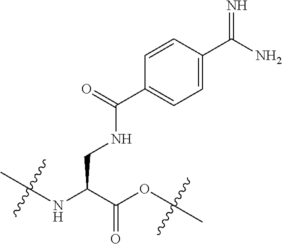 [SMILES: *N[C@@H](CNC(=O)c1ccc(C(=N)N)cc1)C(=O)O*]
(iii)
SEQ ID NO: 10
XPLIYLBimLLBimG-
wherein X in SEQ ID NO: 10 is a residue derived from 6-aminohexanoic acid (Ahx) and each Bim is a modified amino acid residue according to the following structure: 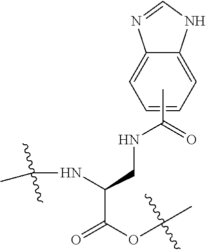
(iv)
SEQ ID NO: 11
XPLIYLmAmLLmAmG-
wherein X SEQ ID NO: 11 is a residue derived from 6-aminohexanoic acid (Ahx) and each mAm is a modified amino acid residue according to the following structure:

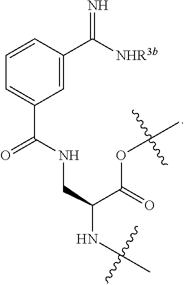

14. A cell penetrating peptide according to claim 1, wherein the cell penetrating peptide is associated with and/or further comprises an agent of interest.

15. A cell penetrating peptide according to claim 14, wherein the agent of interest is selected from the group consisting of a therapeutic agent, a diagnostic agent and a contrast agent, optionally wherein the agent of interest is associated with the peptide via a chemical linkage or via non-covalent interactions.

16. A pharmaceutical composition comprising a cell penetrating peptide according to claim 1 and a therapeutic agent, optionally together with one or more pharmaceutically acceptable excipients, carriers and/or diluents.

17. (canceled)

18. A method of treatment comprising administering a cell penetrating peptide according to claim 1, to a subject in need thereof in a therapeutically effective amount.

19. (canceled)

20. A method of preparing a cell penetrating peptide comprising at least one modified amino acid according to claim 1, wherein the method comprises:
contacting a first amino acid (or peptide fragment comprising a residue of the first amino acid) with a second amino acid (or peptide fragment comprising a residue of the second amino acid) under such conditions so as to promote and/or facilitate a condensation reaction between the first and second amino acids (or peptide fragments comprising residues of the same) to provide a new peptide link,
wherein at least one of the first and second amino acids is the at least one modified amino acid as defined in claim 1 (e.g. as defined in formula (II), (IIa) or (IIb)).

21. (canceled)

22. A method for screening for a cell penetrating peptide comprising:
providing a candidate peptide which comprises at least one modified amino acid residue as defined in claim 1 (e.g. as defined in formula (II), (IIa) or (IIb));
contacting the candidate peptide with a cell; and
determining an effect of the candidate peptide on the cell.

23.-26. (canceled)

27. A modified amino acid according to formula (I″): 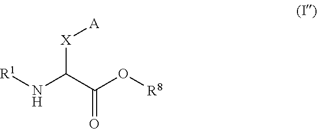
wherein:
R¹ is H or protecting group;
R⁸ is H or protecting group; and
(i) X is selected from optionally substituted C₂-C₄ alkyl, optionally substituted —C₁-C₃ alkyl-NR²(C═O)— and optionally substituted C₁-C₃ alkyl-(C═O)NR²—;
R² is selected from H, optionally substituted C₁-C₆ alkyl and protecting group;
and A is: 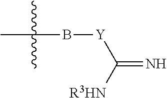
wherein B is selected from optionally substituted aryl, optionally substituted heteroaryl and optionally substituted C₁-C₆ alkyl (such as C₁-C₃ alkyl);
Y is absent;
R³ is selected from H, optionally substituted C₁-C₆ alkyl and protecting group;
or
(ii) X is absent, or is selected from optionally substituted C₁-C₆ alkyl (e.g. C₁-C₃ alkyl), optionally substituted —C₁-C₆ alkyl-NR²(C═O)— (e.g. —C₁-C₃ alkyl-NR²(C═O)—), and optionally substituted C₁-C₆ alkyl-(C═O)NR²— (e.g. C₁-C₃ alkyl-(C═O)NR²—);
R² is selected from H, optionally substituted C₁-C₆ alkyl and protecting group; and
A is selected from: 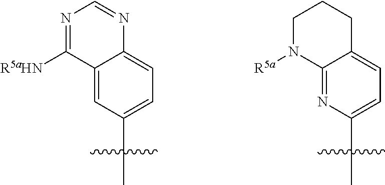
wherein R^{5a} is selected from H and protecting group.

28. A modified amino acid according to claim 27, wherein:
(i) R¹ is H or 9-fluorenylmethyloxycarbonyl (Fmoc) and/or wherein R⁸ is H;
(ii) X is —CH₂NH(C═O)—:
(iii) wherein A is: 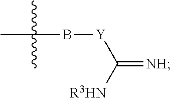
wherein:
B is optionally substituted phenyl or optionally substituted pyridyl;
Y is absent; and
R³ is optionally substituted C₁-C₆ alkyl or an acid labile protecting group; and/or
(iv) A is selected from: 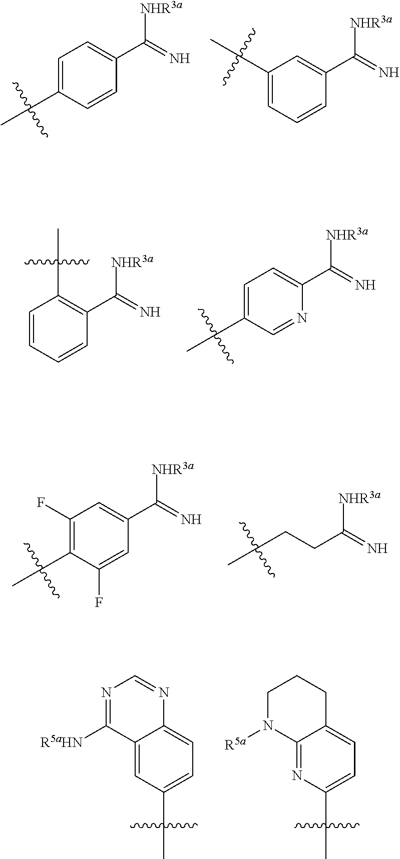
wherein R^{3a} is selected from H, C₁ to C₆ alkyl (e.g. methyl) and acid labile protecting group (e.g. 2,2,4,6,7-pentamethyl-dihydro-benzofuran-5-sulfonyl (Pbf) or Boc); and
R^{5a} is selected from H, C₁ to C₆ alkyl (e.g. methyl) and acid labile protecting group (e.g. Pbf or Boc).

29.-30. (canceled)

31. A modified amino acid according to claim 27, wherein the modified amino acid is represented by formula (Ia″) or formula (Ib″), wherein formula (Ia″) is: 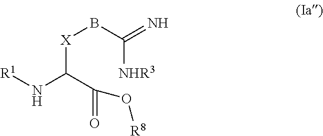
wherein
R¹ is H or a protecting group;
R⁸ is H or a protecting group;
X is selected from optionally substituted C₂-C₄ alkyl, optionally substituted —C₁-C₃ alkyl-NR²(C═O)— and optionally substituted C₁-C₃ alkyl-(C═O)NR²—;
R² is selected from H, optionally substituted C₁-C₆ alkyl and protecting group;
B is selected from optionally substituted aryl and optionally substituted heteroaryl; and
R³ is selected from H, optionally substituted C₁-C₆ alkyl and protecting group; and
wherein formula (Ib″) is: 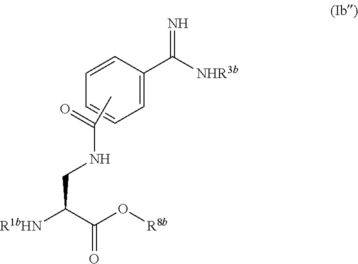
wherein R^{1b} is selected from H and protecting group (e.g. Fmoc); and
R^{3b} is selected from H, C₁-C₆ alkyl and protecting group (e.g. Pbf);
R^{8b} is selected from H and protecting group (e.g. C₁-C₆ alkyl);
wherein the group comprising the amino acid moiety is appended to a carbon atom on the aryl ring at any chemically suitable position.

32.-33. (canceled)

34. A peptide comprising a residue derived from a modified amino acid according to claim 27.
